SEQUENCE LISTING (106 sequences)

SEQ ID NO: 1 - protein, 118 aa, Artificial Sequence
  Synthesized
QVQLQESGGGLVQAGGSLRLSCAASGYIFYGVHMGWYRQAPGKEREFVAAIDLGTTTYYADSVKGRFTISRDNAKNTVYLQMNSLKPEDTAVYYCAVYFDGLTNLPYWGQGTQVTVSS

SEQ ID NO: 2 - protein, 118 aa, Artificial Sequence
  Synthesized
QVQLQESGGGLVQAGGSLRLSCAASGNISVQLDMGWYRQAPGKEREFVAAINQGTTTYYADSVKGRFTISRDNAKNTVYLQMNSLKPEDTAVYYCAVYLYDIWNHPYWGQGTQVTVSS

SEQ ID NO: 3 - protein, 118 aa, Artificial Sequence
  Synthesized
QVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSS

SEQ ID NO: 4 - protein, 118 aa, Artificial Sequence
  Synthesized
QVQLQESGGGLVQAGGSLRLSCAASGTISYYYAMGWYRQAPGKEREFVAAIDLGTSTYYADSVKGRFTISRDNAKNTVYLQMNSLKPEDTAVYYCAAWSDYVTELPYWGQGTQVTVSS

SEQ ID NO: 5 - protein, 118 aa, Artificial Sequence
  Synthesized
QVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSS

SEQ ID NO: 6 - protein, 118 aa, Artificial Sequence
  Synthesized
QVQLQESGGGLVQAGGSLRLSCAASGTISFIGYMGWYRQAPGKERELVASIASGTSTYYADSVKGRFTISRDNAKNTVYLQMNSLKPEDTAVYYCAATQYIQDVHRYWGQGTQVTVSS

SEQ ID NO: 7 - protein, 118 aa, Artificial Sequence
  Synthesized
QVQLQESGGGLVQAGGSLRLSCAASGTISVIGYMGWYRQAPGKERELVASIASGTSTYYADSVKGRFTISRDNAKNTVYLQMNSLKPEDTAVYYCAATHYIQDAHRYWGQGTQVTVSS

SEQ ID NO: 8 - protein, 118 aa, Artificial Sequence
  Synthesized
QVQLQESGGGLVQAGGSLRLSCAASGTISFIGYMGWYRQAPGKERELVASIASGTSTYYADSVKGRFTISRDNAKNTVYLQMNSLKPEDTAVYYCAATQYIQDAHRYWGQGTQVTVSS

SEQ ID NO: 9 - protein, 122 aa, Artificial Sequence
  Synthesized
QVQLQESGGGLVQAGGSLRLSCAASGSIFRLWYMGWYRQAPGKEREFVASIGIGATTNYADSVKGRFTISRDNAKNTVYLQMNSLKPEDTAVYYCAVFGWAYSGYHDDFLYWGQGTQVTVSS

SEQ ID NO: 10 - protein, 122 aa, Artificial Sequence
  Synthesized
QVQLQESGGGLVQAGGSLRLSCAASGTIFDDLLMGWYRQAPGKERELVAAIGAGATTNYADSVKGRFTISRDNAKNTVYLQMNSLKPEDTAVYYCAAWWLYYKGDWALYLYWGQGTQVTVSS

SEQ ID NO: 11 - protein, 118 aa, Artificial Sequence
  Synthesized
QVQLQESGGGLVQAGGSLRLSCAASGTIFGLSDMGWYRQAPGKEREFVAAIAGGTITYYADSVKGRFTISRDNAKNTVYLQMNSLKPEDTAVYYCAAYKYDERYHSYWGQGTQVTVSS

SEQ ID NO: 12 - protein, 118 aa, Artificial Sequence
  Synthesized
QVQLQESGGGLVQAGGSLRLSCAASGSIFWKPVMGWYRQAPGKEREFVAAITSGTNTYYADSVKGRFTISRDNAKNTVYLQMNSLKPEDTAVYYCAVDDYDVVEYPYWGQGTQVTVSS

SEQ ID NO: 13 - protein, 122 aa, Artificial Sequence
  Synthesized
QVQLQESGGGLVQAGGSLRLSCAASGTIFYTLFMGWYRQAPGKEREFVAGIGRGGITYYADSVKGRFTISRDNAKNTVYLQMNSLKPEDTAVYYCAVWYDFLHSDSQAYSYWGQGTQVTVSS

SEQ ID NO: 14 - protein, 121 aa, Artificial Sequence
  Synthesized
QVQLQESGGGLVQAGGSLRLSCAASGTISYAHIMGWYRQAPGKERELVAGISQGSITNYADSVKGRFTISRDNAKNTVYLQMNSLKPEDTAVYYCAVISYDYIKSVPFRYWGQGTQVTVSS

SEQ ID NO: 15 - protein, 119 aa, Artificial Sequence
  Synthesized
DVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSS

SEQ ID NO: 16 - protein, 124 aa, Artificial Sequence
  Synthesized
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 17 - protein, 126 aa, Artificial Sequence
  Synthesized
VQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

SEQ ID NO: 18 - protein, 74 aa, Artificial Sequence
  Synthesized
GDTLGASWHRPDKCCLGYQKRPLPQVLLSSWYPTSQLCSKPGVIFLTKRGRQVCADKSKDWVKKLMQQLPVTAR

SEQ ID NO: 19 - protein, 65 aa, Artificial Sequence
  Synthesized
LTCVKSNSIWFPTSEDCPDGQNLCFKRWQYISPRMYDFTRGCAATCPKAEYRDVINCCGTDKCNK

SEQ ID NO: 20 - protein, 65 aa, Artificial Sequence
  Synthesized
LTCVKSNSIRFPTSGDCPDGQNLCFKRWQSPGMPRPMWALVCAATCPKAPPNEDINCCGTDKCNK

SEQ ID NO: 21 - protein, 249 aa, Artificial Sequence
  Synthesized
DIVLTQSTLPLPVSPGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGSTRSSSSGGGGSGGGGEVQLQESGPGLVKPSQSLSLTCSVTGYSITSSYRWNWIRQFPGNRLEWMGYINSAGISNYNPSLKRRISITRDTSKNQFFLQVNSVTTEDTATYYCARSDNMGTTPFTYWGQGTLVTVSS

SEQ ID NO: 22 - protein, 245 aa, Artificial Sequence
  Synthesized
EVMLVESGGVLVKPGGSLKLSCAASGFTFSRYAMSWVRQTPEKRLEWVATISSGGSYSYYPDSVKGRFTISRDNVKNTLYLQMSSLRSEDTAMYYCARDSGGFAYWGQWGQGTLVTVSAGGGGSGGGGSGGGGSDIQMTQTTSSLSASLGDRVTISCRASQDISNYLNWYQQKPDGTVKLLIYYTSRLHSGVTSRFSGSGSGTDYSLTISNLEQEDIATYFCQQGNTLPWTFGGGTKKVEIKRAD

SEQ ID NO: 23 - protein, 244 aa, Artificial Sequence
  Synthesized
QVQLQESGGGLVQAGGSLRLSCAASGNISVQLDMGWYRQAPGKEREFVAAINQGTTTYYADSVKGRFTISRDNAKNTVYLQMNSLKPEDTAVYYCAVYLYDIWNHPYWGQGTQVTVSSLEVLFQGPQVQLQESGGGLVQAGGSLRLSCAASGNISVQLDMGWYRQAPGKEREFVAAINQGTTTYYADSVKGRFTISRDNAKNTVYLQMNSLKPEDTAVYYCAVYLYDIWNHPYWGQGTQVTVSS

SEQ ID NO: 24 - protein, 244 aa, Artificial Sequence
  Synthesized
QVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSSLEVLFQGPQVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSS

SEQ ID NO: 25 - protein, 245 aa, Artificial Sequence
  Synthesized
DVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSSLEVLFQGPQVQLQESGGGLVQAGGSLRLSCAASGNISVQLDMGWYRQAPGKEREFVAAINQGTTTYYADSVKGRFTISRDNAKNTVYLQMNSLKPEDTAVYYCAVYLYDIWNHPYWGQGTQVTVSS

SEQ ID NO: 26 - protein, 245 aa, Artificial Sequence
  Synthesized
DVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSSLEVLFQGPQVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSS

SEQ ID NO: 27 - protein, 245 aa, Artificial Sequence
  Synthesized
QVQLQESGGGLVQAGGSLRLSCAASGNISVQLDMGWYRQAPGKEREFVAAINQGTTTYYADSVKGRFTISRDNAKNTVYLQMNSLKPEDTAVYYCAVYLYDIWNHPYWGQGTQVTVSSLEVLFQGPDVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSS

SEQ ID NO: 28 - protein, 245 aa, Artificial Sequence
  Synthesized
QVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSSLEVLFQGPDVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSS

SEQ ID NO: 29 - protein, 250 aa, Artificial Sequence
  Synthesized
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSLEVLFQGPQVQLQESGGGLVQAGGSLRLSCAASGNISVQLDMGWYRQAPGKEREFVAAINQGTTTYYADSVKGRFTISRDNAKNTVYLQMNSLKPEDTAVYYCAVYLYDIWNHPYWGQGTQVTVSS

SEQ ID NO: 30 - protein, 250 aa, Artificial Sequence
  Synthesized
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSLEVLFQGPQVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSS

SEQ ID NO: 31 - protein, 252 aa, Artificial Sequence
  Synthesized
VQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSLEVLFQGPQVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSS

SEQ ID NO: 32 - protein, 253 aa, Artificial Sequence
  Synthesized
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSLEVLFQGPQVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSS

SEQ ID NO: 33 - protein, 250 aa, Artificial Sequence
  Synthesized
QVQLQESGGGLVQAGGSLRLSCAASGNISVQLDMGWYRQAPGKEREFVAAINQGTTTYYADSVKGRFTISRDNAKNTVYLQMNSLKPEDTAVYYCAVYLYDIWNHPYWGQGTQVTVSSLEVLFQGPQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 34 - protein, 250 aa, Artificial Sequence
  Synthesized
QVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSSLEVLFQGPQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 35 - protein, 253 aa, Artificial Sequence
  Synthesized
QVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSSLEVLFQGPEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

SEQ ID NO: 36 - protein, 253 aa, Artificial Sequence; identical to SEQ ID NO: 35
  Synthesized

SEQ ID NO: 37 - protein, 245 aa, Artificial Sequence
  Synthesized
DVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSSLEVLFQGPQVQLQESGGGLVQAGGSLRLSCAASGTISFIGYMGWYRQAPGKERELVASIASGTSTYYADSVKGRFTISRDNAKNTVYLQMNSLKPEDTAVYYCAATQYIQDVHRYWGQGTQVTVSS

SEQ ID NO: 38 - protein, 245 aa, Artificial Sequence
  Synthesized
DVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSSLEVLFQGPQVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSS

SEQ ID NO: 39 - protein, 245 aa, Artificial Sequence
  Synthesized
QVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSSLEVLFQGPDVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSS

SEQ ID NO: 40 - protein, 245 aa, Artificial Sequence
  Synthesized
QVQLQESGGGLVQAGGSLRLSCAASGTISFIGYMGWYRQAPGKERELVASIASGTSTYYADSVKGRFTISRDNAKNTVYLQMNSLKPEDTAVYYCAATQYIQDVHRYWGQGTQVTVSSLEVLFQGPDVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSS

SEQ ID NO: 41 - protein, 250 aa, Artificial Sequence
  Synthesized
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSLEVLFQGPQVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSS

SEQ ID NO: 42 - protein, 250 aa, Artificial Sequence
  Synthesized
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSLEVLFQGPQVQLQESGGGLVQAGGSLRLSCAASGTISFIGYMGWYRQAPGKERELVASIASGTSTYYADSVKGRFTISRDNAKNTVYLQMNSLKPEDTAVYYCAATQYIQDVHRYWGQGTQVTVSS

SEQ ID NO: 43 - protein, 253 aa, Artificial Sequence
  Synthesized
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSLEVLFQGPQVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSS

SEQ ID NO: 44 - protein, 253 aa, Artificial Sequence
  Synthesized
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSLEVLFQGPQVQLQESGGGLVQAGGSLRLSCAASGTISFIGYMGWYRQAPGKERELVASIASGTSTYYADSVKGRFTISRDNAKNTVYLQMNSLKPEDTAVYYCAATQYIQDVHRYWGQGTQVTVSS

SEQ ID NO: 45 - protein, 250 aa, Artificial Sequence
  Synthesized
QVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSSLEVLFQGPQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 46 - protein, 250 aa, Artificial Sequence
  Synthesized
QVQLQESGGGLVQAGGSLRLSCAASGTISFIGYMGWYRQAPGKERELVASIASGTSTYYADSVKGRFTISRDNAKNTVYLQMNSLKPEDTAVYYCAATQYIQDVHRYWGQGTQVTVSSLEVLFQGPQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 47 - protein, 253 aa, Artificial Sequence
  Synthesized
QVQLQESGGGLVQAGGSLRLSCAASGTISFIGYMGWYRQAPGKERELVASIASGTSTYYADSVKGRFTISRDNAKNTVYLQMNSLKPEDTAVYYCAATQYIQDVHRYWGQGTQVTVSSLEVLFQGPEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

SEQ ID NO: 48 - protein, 253 aa, Artificial Sequence
  Synthesized
QVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSSLEVLFQGPEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

SEQ ID NO: 49 - protein, 200 aa, Artificial Sequence
  Synthesized
GDTLGASWHRPDKCCLGYQKRPLPQVLLSSWYPTSQLCSKPGVIFLTKRGRQVCADKSKDWVKKLMQQLPVTARLEVLFQGPQVQLQESGGGLVQAGGSLRLSCAASGNISVQLDMGWYRQAPGKEREFVAAINQGTTTYYADSVKGRFTISRDNAKNTVYLQMNSLKPEDTAVYYCAVYLYDIWNHPYWGQGTQVTVSS

SEQ ID NO: 50 - protein, 200 aa, Artificial Sequence
  Synthesized
GDTLGASWHRPDKCCLGYQKRPLPQVLLSSWYPTSQLCSKPGVIFLTKRGRQVCADKSKDWVKKLMQQLPVTARLEVLFQGPQVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSS

SEQ ID NO: 51 - protein, 200 aa, Artificial Sequence
  Synthesized
GDTLGASWHRPDKCCLGYQKRPLPQVLLSSWYPTSQLCSKPGVIFLTKRGRQVCADKSKDWVKKLMQQLPVTARLEVLFQGPQVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSS

SEQ ID NO: 52 - protein, 200 aa, Artificial Sequence
  Synthesized
GDTLGASWHRPDKCCLGYQKRPLPQVLLSSWYPTSQLCSKPGVIFLTKRGRQVCADKSKDWVKKLMQQLPVTARLEVLFQGPQVQLQESGGGLVQAGGSLRLSCAASGTISFIGYMGWYRQAPGKERELVASIASGTSTYYADSVKGRFTISRDNAKNTVYLQMNSLKPEDTAVYYCAATQYIQDVHRYWGQGTQVTVSS

SEQ ID NO: 53 - protein, 191 aa, Artificial Sequence
  Synthesized
QVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSSLEVLFQGPLTCVKSNSIWFPTSEDCPDGQNLCFKRWQYISPRMYDFTRGCAATCPKAEYRDVINCCGTDKCNK

SEQ ID NO: 54 - protein, 191 aa, Artificial Sequence
  Synthesized
LTCVKSNSIWFPTSEDCPDGQNLCFKRWQYISPRMYDFTRGCAATCPKAEYRDVINCCGTDKCNKLEVLFQGPQVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSS

SEQ ID NO: 55 - protein, 191 aa, Artificial Sequence
  Synthesized
QVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSSLEVLFQGPLTCVKSNSIRFPTSGDCPDGQNLCFKRWQSPGMPRPMWALVCAATCPKAPPNEDINCCGTDKCNK

SEQ ID NO: 56 - protein, 191 aa, Artificial Sequence
  Synthesized
LTCVKSNSIRFPTSGDCPDGQNLCFKRWQSPGMPRPMWALVCAATCPKAPPNEDINCCGTDKCNKLEVLFQGPQVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSS

SEQ ID NO: 57 - protein, 379 aa, Artificial Sequence
  Synthesized
QVQLQESGGGLVQAGGSLRLSCAASGNISVQLDMGWYRQAPGKEREFVAAINQGTTTYYADSVKGRFTISRDNAKNTVYLQMNSLKPEDTAVYYCAVYLYDIWNHPYWGQGTQVTVSSGSLEVLFQGPGSDIVLTQSTLPLPVSPGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGSTRSSSSGGGGSGGGGEVQLQESGPGLVKPSQSLSLTCSVTGYSITSSYRWNWIRQFPGNRLEWMGYINSAGISNYNPSLKRRISITRDTSKNQFFLQVNSVTTEDTATYYCARSDNMGTTPFTYWGQGTLVTVSS

SEQ ID NO: 58 - protein, 379 aa, Artificial Sequence
  Synthesized
QVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSSGSLEVLFQGPGSDIVLTQSTLPLPVSPGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGSTRSSSSGGGGSGGGGEVQLQESGPGLVKPSQSLSLTCSVTGYSITSSYRWNWIRQFPGNRLEWMGYINSAGISNYNPSLKRRISITRDTSKNQFFLQVNSVTTEDTATYYCARSDNMGTTPFTYWGQGTLVTVSS

SEQ ID NO: 59 - protein, 379 aa, Artificial Sequence
  Synthesized
DIVLTQSTLPLPVSPGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGSTRSSSSGGGGSGGGGEVQLQESGPGLVKPSQSLSLTCSVTGYSITSSYRWNWIRQFPGNRLEWMGYINSAGISNYNPSLKRRISITRDTSKNQFFLQVNSVTTEDTATYYCARSDNMGTTPFTYWGQGTLVTVSSGSLEVLFQGPGSQVQLQESGGGLVQAGGSLRLSCAASGNISVQLDMGWYRQAPGKEREFVAAINQGTTTYYADSVKGRFTISRDNAKNTVYLQMNSLKPEDTAVYYCAVYLYDIWNHPYWGQGTQVTVSS

SEQ ID NO: 60 - protein, 379 aa, Artificial Sequence
  Synthesized
DIVLTQSTLPLPVSPGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGSTRSSSSGGGGSGGGGEVQLQESGPGLVKPSQSLSLTCSVTGYSITSSYRWNWIRQFPGNRLEWMGYINSAGISNYNPSLKRRISITRDTSKNQFFLQVNSVTTEDTATYYCARSDNMGTTPFTYWGQGTLVTVSSGSLEVLFQGPGSQVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSS

SEQ ID NO: 61 - protein, 379 aa, Artificial Sequence
  Synthesized
QVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSSGSLEVLFQGPGSDIVLTQSTLPLPVSPGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGSTRSSSSGGGGSGGGGEVQLQESGPGLVKPSQSLSLTCSVTGYSITSSYRWNWIRQFPGNRLEWMGYINSAGISNYNPSLKRRISITRDTSKNQFFLQVNSVTTEDTATYYCARSDNMGTTPFTYWGQGTLVTVSS

SEQ ID NO: 62 - protein, 379 aa, Artificial Sequence
  Synthesized
QVQLQESGGGLVQAGGSLRLSCAASGTISFIGYMGWYRQAPGKERELVASIASGTSTYYADSVKGRFTISRDNAKNTVYLQMNSLKPEDTAVYYCAATQYIQDVHRYWGQGTQVTVSSGSLEVLFQGPGSDIVLTQSTLPLPVSPGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGSTRSSSSGGGGSGGGGEVQLQESGPGLVKPSQSLSLTCSVTGYSITSSYRWNWIRQFPGNRLEWMGYINSAGISNYNPSLKRRISITRDTSKNQFFLQVNSVTTEDTATYYCARSDNMGTTPFTYWGQGTLVTVSS

SEQ ID NO: 63 - protein, 379 aa, Artificial Sequence
  Synthesized
DIVLTQSTLPLPVSPGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGSTRSSSSGGGGSGGGGEVQLQESGPGLVKPSQSLSLTCSVTGYSITSSYRWNWIRQFPGNRLEWMGYINSAGISNYNPSLKRRISITRDTSKNQFFLQVNSVTTEDTATYYCARSDNMGTTPFTYWGQGTLVTVSSGSLEVLFQGPGSQVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSS

SEQ ID NO: 64 - protein, 379 aa, Artificial Sequence
  Synthesized
DIVLTQSTLPLPVSPGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGSTRSSSSGGGGSGGGGEVQLQESGPGLVKPSQSLSLTCSVTGYSITSSYRWNWIRQFPGNRLEWMGYINSAGISNYNPSLKRRISITRDTSKNQFFLQVNSVTTEDTATYYCARSDNMGTTPFTYWGQGTLVTVSSGSLEVLFQGPGSQVQLQESGGGLVQAGGSLRLSCAASGTISFIGYMGWYRQAPGKERELVASIASGTSTYYADSVKGRFTISRDNAKNTVYLQMNSLKPEDTAVYYCAATQYIQDVHRYWGQGTQVTVSS

SEQ ID NO: 65 - protein, 371 aa, Artificial Sequence
  Synthesized
EVMLVESGGVLVKPGGSLKLSCAASGFTFSRYAMSWVRQTPEKRLEWVATISSGGSYSYYPDSVKGRFTISRDNVKNTLYLQMSSLRSEDTAMYYCARDSGGFAYWGQWGQGTLVTVSAGGGGSGGGGSGGGGSDIQMTQTTSSLSASLGDRVTISCRASQDISNYLNWYQQKPDGTVKLLIYYTSRLHSGVTSRFSGSGSGTDYSLTISNLEQEDIATYFCQQGNTLPWTFGGGTKKVEIKRADLEVLFQGPQVQLQESGGGLVQAGGSLRLSCAASGTISFIGYMGWYRQAPGKERELVASIASGTSTYYADSVKGRFTISRDNAKNTVYLQMNSLKPEDTAVYYCAATQYIQDVHRYWGQGTQVTVSS

SEQ ID NO: 66 - protein, 371 aa, Artificial Sequence
  Synthesized
EVMLVESGGVLVKPGGSLKLSCAASGFTFSRYAMSWVRQTPEKRLEWVATISSGGSYSYYPDSVKGRFTISRDNVKNTLYLQMSSLRSEDTAMYYCARDSGGFAYWGQWGQGTLVTVSAGGGGSGGGGSGGGGSDIQMTQTTSSLSASLGDRVTISCRASQDISNYLNWYQQKPDGTVKLLIYYTSRLHSGVTSRFSGSGSGTDYSLTISNLEQEDIATYFCQQGNTLPWTFGGGTKKVEIKRADLEVLFQGPQVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSS

SEQ ID NO: 67 - protein, 371 aa, Artificial Sequence
  Synthesized
QVQLQESGGGLVQAGGSLRLSCAASGTISFIGYMGWYRQAPGKERELVASIASGTSTYYADSVKGRFTISRDNAKNTVYLQMNSLKPEDTAVYYCAATQYIQDVHRYWGQGTQVTVSSLEVLFQGPEVMLVESGGVLVKPGGSLKLSCAASGFTFSRYAMSWVRQTPEKRLEWVATISSGGSYSYYPDSVKGRFTISRDNVKNTLYLQMSSLRSEDTAMYYCARDSGGFAYWGQWGQGTLVTVSAGGGGSGGGGSGGGGSDIQMTQTTSSLSASLGDRVTISCRASQDISNYLNWYQQKPDGTVKLLIYYTSRLHSGVTSRFSGSGSGTDYSLTISNLEQEDIATYFCQQGNTLPWTFGGGTKKVEIKRAD

SEQ ID NO: 68 - protein, 371 aa, Artificial Sequence
  Synthesized
QVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSSLEVLFQGPEVMLVESGGVLVKPGGSLKLSCAASGFTFSRYAMSWVRQTPEKRLEWVATISSGGSYSYYPDSVKGRFTISRDNVKNTLYLQMSSLRSEDTAMYYCARDSGGFAYWGQWGQGTLVTVSAGGGGSGGGGSGGGGSDIQMTQTTSSLSASLGDRVTISCRASQDISNYLNWYQQKPDGTVKLLIYYTSRLHSGVTSRFSGSGSGTDYSLTISNLEQEDIATYFCQQGNTLPWTFGGGTKKVEIKRAD

SEQ ID NO: 69 - protein, 371 aa, Artificial Sequence
  Synthesized
EVMLVESGGVLVKPGGSLKLSCAASGFTFSRYAMSWVRQTPEKRLEWVATISSGGSYSYYPDSVKGRFTISRDNVKNTLYLQMSSLRSEDTAMYYCARDSGGFAYWGQWGQGTLVTVSAGGGGSGGGGSGGGGSDIQMTQTTSSLSASLGDRVTISCRASQDISNYLNWYQQKPDGTVKLLIYYTSRLHSGVTSRFSGSGSGTDYSLTISNLEQEDIATYFCQQGNTLPWTFGGGTKKVEIKRADLEVLFQGPQVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSS

SEQ ID NO: 70 - protein, 371 aa, Artificial Sequence; identical to SEQ ID NO: 65
  Synthesized

SEQ ID NO: 71 - protein, 371 aa, Artificial Sequence
  Synthesized
QVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSSLEVLFQGPEVMLVESGGVLVKPGGSLKLSCAASGFTFSRYAMSWVRQTPEKRLEWVATISSGGSYSYYPDSVKGRFTISRDNVKNTLYLQMSSLRSEDTAMYYCARDSGGFAYWGQWGQGTLVTVSAGGGGSGGGGSGGGGSDIQMTQTTSSLSASLGDRVTISCRASQDISNYLNWYQQKPDGTVKLLIYYTSRLHSGVTSRFSGSGSGTDYSLTISNLEQEDIATYFCQQGNTLPWTFGGGTKKVEIKRAD

SEQ ID NO: 72 - protein, 371 aa, Artificial Sequence; identical to SEQ ID NO: 67
  Synthesized

SEQ ID NO: 73 - protein, 8 aa, Artificial Sequence
  Synthesized
LEVLFQGP

SEQ ID NO: 74 - protein, 249 aa, Artificial Sequence
  Synthesized
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVKDRVAVAGKGSYYFDSWGRGTTVTVSSGGGGSGGGGSGGGGSQSVLTQPPSVSEAPGQRVTIACSGSSSNIGNNAVSWYQQLPGKAPTLLIYYDNLLPSGVSDRFSGSKSGTSASLAISGLQSEDEADYYCAAWDDSLNDWVFGGGTKVTVL

SEQ ID NO: 75 - protein, 379 aa, Artificial Sequence
  Synthesized
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVKDRVAVAGKGSYYFDSWGRGTTVTVSSGGGGSGGGGSGGGGSQSVLTQPPSVSEAPGQRVTIACSGSSSNIGNNAVSWYQQLPGKAPTLLIYYDNLLPSGVSDRFSGSKSGTSASLAISGLQSEDEADYYCAAWDDSLNDWVFGGGTKVTVLGSLEVLFQGPGSQVQLQESGGGLVQAGGSLRLSCAASGNISVQLDMGWYRQAPGKEREFVAAINQGTTTYYADSVKGRFTISRDNAKNTVYLQMNSLKPEDTAVYYCAVYLYDIWNHPYWGQGTQVTVSS

SEQ ID NO: 76 - protein, 379 aa, Artificial Sequence
  Synthesized
QVQLQESGGGLVQAGGSLRLSCAASGNISVQLDMGWYRQAPGKEREFVAAINQGTTTYYADSVKGRFTISRDNAKNTVYLQMNSLKPEDTAVYYCAVYLYDIWNHPYWGQGTQVTVSSGSLEVLFQGPGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVKDRVAVAGKGSYYFDSWGRGTTVTVSSGGGGSGGGGSGGGGSQSVLTQPPSVSEAPGQRVTIACSGSSSNIGNNAVSWYQQLPGKAPTLLIYYDNLLPSGVSDRFSGSKSGTSASLAISGLQSEDEADYYCAAWDDSLNDWVFGGGTKVTVL

SEQ ID NO: 77 - protein, 379 aa, Artificial Sequence
  Synthesized
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVKDRVAVAGKGSYYFDSWGRGTTVTVSSGGGGSGGGGSGGGGSQSVLTQPPSVSEAPGQRVTIACSGSSSNIGNNAVSWYQQLPGKAPTLLIYYDNLLPSGVSDRFSGSKSGTSASLAISGLQSEDEADYYCAAWDDSLNDWVFGGGTKVTVLGSLEVLFQGPGSQVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSS

SEQ ID NO: 78 - protein, 379 aa, Artificial Sequence
  Synthesized
QVQLQESGGGLVQAGGSLRLSCAASGSISGGKGMGWYRQAPGKEREFVAAIGSGAITYYADSVKGRFTISRDNAKNTVYLQMNSLKPEDTAVYYCAVYTTALDEYPYWGQGTQVTVSSGSLEVLFQGPGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVKDRVAVAGKGSYYFDSWGRGTTVTVSSGGGGSGGGGSGGGGSQSVLTQPPSVSEAPGQRVTIACSGSSSNIGNNAVSWYQQLPGKAPTLLIYYDNLLPSGVSDRFSGSKSGTSASLAISGLQSEDEADYYCAAWDDSLNDWVFGGGTKVTVL

SEQ ID NO: 79 - protein, 379 aa, Artificial Sequence
  Synthesized
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVKDRVAVAGKGSYYFDSWGRGTTVTVSSGGGGSGGGGSGGGGSQSVLTQPPSVSEAPGQRVTIACSGSSSNIGNNAVSWYQQLPGKAPTLLIYYDNLLPSGVSDRFSGSKSGTSASLAISGLQSEDEADYYCAAWDDSLNDWVFGGGTKVTVLGSLEVLFQGPGSQVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSS

SEQ ID NO: 80 - protein, 379 aa, Artificial Sequence
  Synthesized
QVQLQESGGGLVQAGGSLRLSCAASGYISGYYVMGWYRQAPGKEREFVASISYGASTYYADSVKGRFTISRDNAKNTVYLQMNSLKPEDTAVYYCAVDFDSNYAHTYWGQGTQVTVSSGSLEVLFQGPGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVKDRVAVAGKGSYYFDSWGRGTTVTVSSGGGGSGGGGSGGGGSQSVLTQPPSVSEAPGQRVTIACSGSSSNIGNNAVSWYQQLPGKAPTLLIYYDNLLPSGVSDRFSGSKSGTSASLAISGLQSEDEADYYCAAWDDSLNDWVFGGGTKVTVL

SEQ ID NO: 81 - protein, 379 aa, Artificial Sequence
  Synthesized
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVKDRVAVAGKGSYYFDSWGRGTTVTVSSGGGGSGGGGSGGGGSQSVLTQPPSVSEAPGQRVTIACSGSSSNIGNNAVSWYQQLPGKAPTLLIYYDNLLPSGVSDRFSGSKSGTSASLAISGLQSEDEADYYCAAWDDSLNDWVFGGGTKVTVLGSLEVLFQGPGSQVQLQESGGGLVQAGGSLRLSCAASGTISFIGYMGWYRQAPGKERELVASIASGTSTYYADSVKGRFTISRDNAKNTVYLQMNSLKPEDTAVYYCAATQYIQDVHRYWGQGTQVTVSS

SEQ ID NO: 82 - protein, 379 aa, Artificial Sequence
  Synthesized
QVQLQESGGGLVQAGGSLRLSCAASGTISFIGYMGWYRQAPGKERELVASIASGTSTYYADSVKGRFTISRDNAKNTVYLQMNSLKPEDTAVYYCAATQYIQDVHRYWGQGTQVTVSSGSLEVLFQGPGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVKDRVAVAGKGSYYFDSWGRGTTVTVSSGGGGSGGGGSGGGGSQSVLTQPPSVSEAPGQRVTIACSGSSSNIGNNAVSWYQQLPGKAPTLLIYYDNLLPSGVSDRFSGSKSGTSASLAISGLQSEDEADYYCAAWDDSLNDWVFGGGTKVTVL

SEQ ID NO: 83 - protein, 383 aa, Artificial Sequence
  Synthesized
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVKDRVAVAGKGSYYFDSWGRGTTVTVSSGGGGSGGGGSGGGGSQSVLTQPPSVSEAPGQRVTIACSGSSSNIGNNAVSWYQQLPGKAPTLLIYYDNLLPSGVSDRFSGSKSGTSASLAISGLQSEDEADYYCAAWDDSLNDWVFGGGTKVTVLGSLEVLFQGPGSQVQLQESGGGLVQAGGSLRLSCAASGSIFRLWYMGWYRQAPGKEREFVASIGIGATTNYADSVKGRFTISRDNAKNTVYLQMNSLKPEDTAVYYCAVFGWAYSGYHDDFLYWGQGTQVTVSS

SEQ ID NO: 84 - protein, 383 aa, Artificial Sequence
  Synthesized
QVQLQESGGGLVQAGGSLRLSCAASGSIFRLWYMGWYRQAPGKEREFVASIGIGATTNYADSVKGRFTISRDNAKNTVYLQMNSLKPEDTAVYYCAVFGWAYSGYHDDFLYWGQGTQVTVSSGSLEVLFQGPGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVKDRVAVAGKGSYYFDSWGRGTTVTVSSGGGGSGGGGSGGGGSQSVLTQPPSVSEAPGQRVTIACSGSSSNIGNNAVSWYQQLPGKAPTLLIYYDNLLPSGVSDRFSGSKSGTSASLAISGLQSEDEADYYCAAWDDSLNDWVFGGGTKVTVL

SEQ ID NO: 85 - protein, 119 aa, Artificial Sequence; identical to SEQ ID NO: 15
  Synthesized

SEQ ID NO: 86 - protein, 249 aa, Artificial Sequence; identical to SEQ ID NO: 21
  Synthesized

SEQ ID NO: 87 - protein, 124 aa, Artificial Sequence; identical to SEQ ID NO: 16
  Synthesized

SEQ ID NO: 88 - protein, 127 aa, Artificial Sequence
  Synthesized
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

SEQ ID NO: 89 - protein, 249 aa, Artificial Sequence
  Synthesized
DVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSSLEVLFQGPQVQLQESGGGLVQAGGSLRLSCAASGSIFRLWYMGWYRQAPGKEREFVASIGIGATTNYADSVKGRFTISRDNAKNTVYLQMNSLKPEDTAVYYCAVFGWAYSGYHDDFLYWGQGTQVTVSS

SEQ ID NO: 90 - protein, 249 aa, Artificial Sequence
  Synthesized
QVQLQESGGGLVQAGGSLRLSCAASGSIFRLWYMGWYRQAPGKEREFVASIGIGATTNYADSVKGRFTISRDNAKNTVYLQMNSLKPEDTAVYYCAVFGWAYSGYHDDFLYWGQGTQVTVSSLEVLFQGPDVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSS

SEQ ID NO: 91 - protein, 254 aa, Artificial Sequence
  Synthesized
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSLEVLFQGPQVQLQESGGGLVQAGGSLRLSCAASGSIFRLWYMGWYRQAPGKEREFVASIGIGATTNYADSVKGRFTISRDNAKNTVYLQMNSLKPEDTAVYYCAVFGWAYSGYHDDFLYWGQGTQVTVSS

SEQ ID NO: 92 - protein, 254 aa, Artificial Sequence
  Synthesized
QVQLQESGGGLVQAGGSLRLSCAASGSIFRLWYMGWYRQAPGKEREFVASIGIGATTNYADSVKGRFTISRDNAKNTVYLQMNSLKPEDTAVYYCAVFGWAYSGYHDDFLYWGQGTQVTVSSLEVLFQGPQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 93 - protein, 257 aa, Artificial Sequence
  Synthesized
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSLEVLFQGPQVQLQESGGGLVQAGGSLRLSCAASGSIFRLWYMGWYRQAPGKEREFVASIGIGATTNYADSVKGRFTISRDNAKNTVYLQMNSLKPEDTAVYYCAVFGWAYSGYHDDFLYWGQGTQVTVSS

SEQ ID NO: 94 - protein, 257 aa, Artificial Sequence
  Synthesized
QVQLQESGGGLVQAGGSLRLSCAASGSIFRLWYMGWYRQAPGKEREFVASIGIGATTNYADSVKGRFTISRDNAKNTVYLQMNSLKPEDTAVYYCAVFGWAYSGYHDDFLYWGQGTQVTVSSLEVLFQGPEVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

SEQ ID NO: 95 - protein, 383 aa, Artificial Sequence
  Synthesized
DIVLTQSTLPLPVSPGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGSTRSSSSGGGGSGGGGEVQLQESGPGLVKPSQSLSLTCSVTGYSITSSYRWNWIRQFPGNRLEWMGYINSAGISNYNPSLKRRISITRDTSKNQFFLQVNSVTTEDTATYYCARSDNMGTTPFTYWGQGTLVTVSSGSLEVLFQGPGSQVQLQESGGGLVQAGGSLRLSCAASGSIFRLWYMGWYRQAPGKEREFVASIGIGATTNYADSVKGRFTISRDNAKNTVYLQMNSLKPEDTAVYYCAVFGWAYSGYHDDFLYWGQGTQVTVSS

SEQ ID NO: 96 - protein, 383 aa, Artificial Sequence
  Synthesized
QVQLQESGGGLVQAGGSLRLSCAASGSIFRLWYMGWYRQAPGKEREFVASIGIGATTNYADSVKGRFTISRDNAKNTVYLQMNSLKPEDTAVYYCAVFGWAYSGYHDDFLYWGQGTQVTVSSGSLEVLFQGPGSDIVLTQSTLPLPVSPGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKGGSTRSSSSGGGGSGGGGEVQLQESGPGLVKPSQSLSLTCSVTGYSITSSYRWNWIRQFPGNRLEWMGYINSAGISNYNPSLKRRISITRDTSKNQFFLQVNSVTTEDTATYYCARSDNMGTTPFTYWGQGTLVTVSS

SEQ ID NO: 97 - protein, 256 aa, Artificial Sequence
  Synthesized
QVQLQESGGGLVQAGGSLRLSCAASGTIFDDLLMGWYRQAPGKERELVAAIGAGATTNYADSVKGRFTISRDNAKNTVYLQMNSLKPEDTAVYYCAAWWLYYKGDWALYLYWGQGTQVTVSSGSLEVLFQGPGSQVQLQESGGGLVQAGGSLRLSCAASGTIFDDLLMGWYRQAPGKERELVAAIGAGATTNYADSVKGRFTISRDNAKNTVYLQMNSLKPEDTAVYYCAAWWLYYKGDWALYLYWGQGTQVTVSS

SEQ ID NO: 98 - protein, 248 aa, Artificial Sequence
  Synthesized
QVQLQESGGGLVQAGGSLRLSCAASGTIFGLSDMGWYRQAPGKEREFVAAIAGGTITYYADSVKGRFTISRDNAKNTVYLQMNSLKPEDTAVYYCAAYKYDERYHSYWGQGTQVTVSSGSLEVLFQGPGSQVQLQESGGGLVQAGGSLRLSCAASGTIFGLSDMGWYRQAPGKEREFVAAIAGGTITYYADSVKGRFTISRDNAKNTVYLQMNSLKPEDTAVYYCAAYKYDERYHSYWGQGTQVTVSS

SEQ ID NO: 99 - protein, 248 aa, Artificial Sequence
  Synthesized
QVQLQESGGGLVQAGGSLRLSCAASGSIFWKPVMGWYRQAPGKEREFVAAITSGTNTYYADSVKGRFTISRDNAKNTVYLQMNSLKPEDTAVYYCAVDDYDVVEYPYWGQGTQVTVSSGSLEVLFQGPGSQVQLQESGGGLVQAGGSLRLSCAASGSIFWKPVMGWYRQAPGKEREFVAAITSGTNTYYADSVKGRFTISRDNAKNTVYLQMNSLKPEDTAVYYCAVDDYDVVEYPYWGQGTQVTVSS

SEQ ID NO: 100 - protein, 256 aa, Artificial Sequence
  Synthesized
QVQLQESGGGLVQAGGSLRLSCAASGTIFYTLFMGWYRQAPGKEREFVAGIGRGGITYYADSVKGRFTISRDNAKNTVYLQMNSLKPEDTAVYYCAVWYDFLHSDSQAYSYWGQGTQVTVSSGSLEVLFQGPGSQVQLQESGGGLVQAGGSLRLSCAASGTIFYTLFMGWYRQAPGKEREFVAGIGRGGITYYADSVKGRFTISRDNAKNTVYLQMNSLKPEDTAVYYCAVWYDFLHSDSQAYSYWGQGTQVTVSS

SEQ ID NO: 101 - protein, 254 aa, Artificial Sequence
  Synthesized
QVQLQESGGGLVQAGGSLRLSCAASGTISYAHIMGWYRQAPGKERELVAGISQGSITNYADSVKGRFTISRDNAKNTVYLQMNSLKPEDTAVYYCAVISYDYIKSVPFRYWGQGTQVTVSSGSLEVLFQGPGSQVQLQESGGGLVQAGGSLRLSCAASGTISYAHIMGWYRQAPGKERELVAGISQGSITNYADSVKGRFTISRDNAKNTVYLQMNSLKPEDTAVYYCAVISYDYIKSVPFRYWGQGTQVTVSS

SEQ ID NO: 102 - protein, 251 aa, Artificial Sequence
  Synthesized
QVQLQESGGGLVQAGGSLRLSCAASGTIFGLSDMGWYRQAPGKEREFVAAIAGGTITYYADSVKGRFTISRDNAKNTVYLQMNSLKPEDTAVYYCAAYKYDERYHSYWGQGTQVTVSSGSLEVLFQGPGSQVQLQESGGGLVQAGGSLRLSCAASGTISYAHIMGWYRQAPGKERELVAGISQGSITNYADSVKGRFTISRDNAKNTVYLQMNSLKPEDTAVYYCAVISYDYIKSVPFRYWGQGTQVTVSS

SEQ ID NO: 103 - protein, 252 aa, Artificial Sequence
  Synthesized
QVQLQESGGGLVQAGGSLRLSCAASGTIFGLSDMGWYRQAPGKEREFVAAIAGGTITYYADSVKGRFTISRDNAKNTVYLQMNSLKPEDTAVYYCAAYKYDERYHSYWGQGTQVTVSSGSLEVLFQGPGSQVQLQESGGGLVQAGGSLRLSCAASGTIFYTLFMGWYRQAPGKEREFVAGIGRGGITYYADSVKGRFTISRDNAKNTVYLQMNSLKPEDTAVYYCAVWYDFLHSDSQAYSYWGQGTQVTVSS

SEQ ID NO: 104 - protein, 251 aa, Artificial Sequence
  Synthesized
QVQLQESGGGLVQAGGSLRLSCAASGSIFWKPVMGWYRQAPGKEREFVAAITSGTNTYYADSVKGRFTISRDNAKNTVYLQMNSLKPEDTAVYYCAVDDYDVVEYPYWGQGTQVTVSSGSLEVLFQGPGSQVQLQESGGGLVQAGGSLRLSCAASGTISYAHIMGWYRQAPGKERELVAGISQGSITNYADSVKGRFTISRDNAKNTVYLQMNSLKPEDTAVYYCAVISYDYIKSVPFRYWGQGTQVTVSS

SEQ ID NO: 105 - protein, 252 aa, Artificial Sequence
  Synthesized
QVQLQESGGGLVQAGGSLRLSCAASGSIFWKPVMGWYRQAPGKEREFVAAITSGTNTYYADSVKGRFTISRDNAKNTVYLQMNSLKPEDTAVYYCAVDDYDVVEYPYWGQGTQVTVSSGSLEVLFQGPGSQVQLQESGGGLVQAGGSLRLSCAASGTIFYTLFMGWYRQAPGKEREFVAGIGRGGITYYADSVKGRFTISRDNAKNTVYLQMNSLKPEDTAVYYCAVWYDFLHSDSQAYSYWGQGTQVTVSS

SEQ ID NO: 106 - protein, 12 aa, Artificial Sequence
  Synthesized
GSLEVLFQGPGS

(19) United States
(12) Patent Application Publication
(10) Pub. No.: US 2024/0239891 A1
(43) Pub. Date: Jul. 18, 2024

(54) RECEPTOR ELIMINATION BY UBIQUITIN LIGASE RECRUITMENT

(71) Applicant: THE BOARD OF TRUSTEES OF THE LELAND STANFORD JUNIOR UNIVERSITY, Stanford, CA (US)

(72) Inventors: Kenan Christopher GARCIA, Stanford, CA (US); Dirk Hubert SIEPE, Stanford, CA (US)

(73) Assignee: THE BOARD OF TRUSTEES OF THE LELAND STANFORD JUNIOR UNIVERSITY, Stanford, CA (US)

(21) Appl. No.: 18/561,688

(22) PCT Filed: May 19, 2022
(86) PCT No.: PCT/US2022/030132

§ 371 Date: Nov. 16, 2023

Related U.S. Application Data
(60) Provisional application No. 63/190,673, filed on May 19, 2021.

(51) Int. Cl. C07K 16/28; A61K 39/00; C07K 16/40

(52) U.S. Cl. CPC C07K 16/2818; C07K 16/2863; C07K 16/40; A61K 39/00; C07K 2317/31; C07K 2317/569; C07K 2319/50

(57) ABSTRACT

Provided herein are Receptor Elimination by E3 Ubiquitin Ligase Recruitment (REULR) constructs and related compositions, kits, methods of use and methods of production. REULR constructs comprise a variable domain heavy chain antibody (VHH) specific for a E3 Ubiquitin Ligase, and a VHH specific for a target protein, where in the E3 Ubiquitin Ligase and the target protein are different. REULR constructs described here target proteins associated with many maladies, but especially cancer.

### CROSS-REFERENCE TO RELATED APPLICATIONS

This application claims priority to U.S. provisional patent application No. 63/190,673, filed May 19, 2021, the contents of which are incorporated herein by reference in their entirety.

### BACKGROUND

The post-translational attachment of ubiquitin (Ub), a highly conserved 76-amino-acid polypeptide, directs myriad eukaryotic proteins to a variety of fates and functions. The process of ubiquitylation includes targeting proteins for 26S proteasome dependent degradation, internalization and lysosomal targeting, modulation of protein interactions, alteration of subcellular distribution, regulation of transcription, DNA repair and modulation of transmembrane signaling cascades. Protein ubiquitylation has been linked to virtually every cellular process, is involved in a multitude of cellular diseases and deregulation of processes involving the ubiquitin system has detrimental cellular consequences. In consequence, E3s are implicated in a number of pathophysiological conditions, which makes them attractive therapeutic targets.

Protein ubiquitylation generally involves covalent attachment of ubiquitin to the protein substrate. Depending on the ubiquitin linkage, either mono, oligo or poly-ubiquitin, the protein substrate may be subsequently degraded by the via the ubiquitin-proteasome pathway (26S proteasome), the lysosomal degradation pathway, or the proteins function is modulated in a ubiquitin dependent but degradation independent fashion, and the free ubiquitin is recycled.

Post-translational modification of proteins by Ub and UbLs regulates almost every aspect of biology and enables complex and reversible regulation of protein stability and activity. Because of this broad role in cell biology, dysfunction in Ub pathway enzymes results in a multitude of human diseases. The attachment of ubiquitin to many known substrate proteins is believed to occur in a series of enzymatic reactions carried out sequentially by three classes of proteins: an ubiquitin-activating enzyme (E1) activates ubiquitin in an ATP-dependent manner to form a thioester bond between the carboxy-terminal Gly of Ub and a Cys residue of E1; activated Ub is then transferred to an ubiquitin-conjugating enzyme (E2 or UBC) to form another thioester bond; and a ubiquitin ligase (E3) catalyzes or promotes, in a substrate specific manner, the transfer of Ub from the E2 to a Lys (K) residue of the substrate protein to form an isopeptide bond.

It is known that The E3 enzymes provide substrate specificity during ubiquitination. Humans contain one form of the E1 enzyme, over 30 E2 enzymes and over 350 E3 enzymes. The E3 enzymes are often grouped into three families based on the presence of the E3 catalytic core domain: the homology to E6AP carboxyl terminus (HECT), the gene (RING) finger (RNF) and the U-box protein families. The mechanics of transfer from E3 to substrate depends on the specific class of E3 ligase involved. These ligases comprise over 500 different proteins and are categorized into multiple classes defined by the structural element of their E3 functional activity. The E3 ligases are grouped into two main classes: the HECT ligases containing an active site cysteine which serves to accept Ub prior to substrate transfer, and the RING E3 ligases, which contain zinc finger-like domains that act as scaffolds enabling transfer of Ub directly from an E2 enzyme to a substrate. The RBR (RING type) is a subclass of E3 Ub ligases that are considered RING/HECT hybrids in that similar to RING ligases coordinate zinc and bind E2, but also contain an active site cysteine similar to HECT ligases. Specifically, both HECT and RING ligases transfer an activated Ub from a thioester to the 8-amino acid group of a lysine residue on a substrate; however, HECT ligases have an active site cysteine that forms an intermediate thioester bond with Ub, while RING ligases function as a scaffold to allow direct Ub transfer from the E2 to substrate. The transmembrane E3 ligase family is another subclass of diverse RING E3 Ub ligases that is minimally defined by three domains, an extracellular domain (ECD), a transmembrane domain™, and a RING-H2 finger (RNF) domain and consists of approximately 50 members including several subfamilies. The human transmembrane RNF E3 ubiquitin ligase family can be further grouped into structurally related families including the RING domain containing proteins (25), tripartite motif containing (TRIM; 2), PA-TM-RING (11), RING between RING (RBR; 5) and the membrane-associated RING-CH (MARCH; 9) families. The PA-TM-RING family includes 11 members: GRAIL (RNF128), GOLIATH (RNF130) RNF133, RNF148, RNF149, RNF150, GODZILLA (RNF167), RNF13, RNF43, ZNRF3 and ZNRF4. The extracellular or luminal PA domain is also found in receptors and peptidases in yeast, metazoans and plants. The PA domain is proposed to serve as a protein-protein interaction module.

The present compositions and methods address needs in the art related to these naturally occurring systems and processes.

### SUMMARY

Provided herein are Receptor Elimination by E3 Ubiquitin Ligase Recruitment (REULR) constructs and related compositions. Such constructs often comprise a variable domain heavy chain antibody (VHH) specific for a E3 Ubiquitin Ligase, and a VHH specific for a target protein, with the E3 Ubiquitin Ligase and the target protein being different proteins.

Also provided herein are REULR construct compositions, with the REULR construct being defined by the following formula:

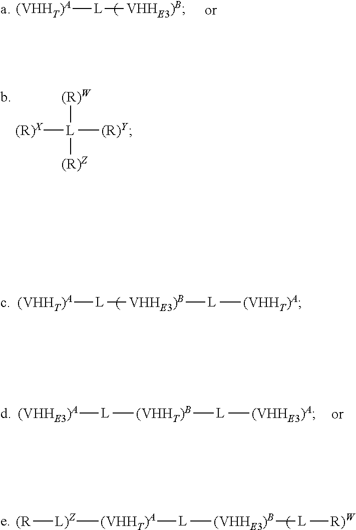

In such formulas defining exemplary REULR constructs, VHH is a variable domain heavy chain antibody specific for a target protein or an E3 Ubiquitin Ligase; R is VHH_{T} or VHH_{E3}; L is a linker molecule; T is a target protein such that the VHH is specific for the target protein; _{E3} is an E3 Ubiquitin Ligase such that the VHH is specific for the E3 Ubiquitin Ligase; ^{A} is 1 or more; ^{B} is 1 or more; ^{w} is 0, or 1 or more; ^{x} is 0, or 1 or more; ^{Y} is 0, or 1 or more; and ^{z} is 0, or 1 or more.

Also provided herein are linker molecules adapted to ligate the VHH specific for the E3 Ubiquitin Ligase to the VHH specific for a target protein in a manner such that the VHH specific for the E3 Ubiquitin Ligase and the VHH specific for a target protein are each independently available and able, respectively, to bind with the E3 Ubiquitin Ligase and the target protein.

In frequently included embodiments, the REULR construct, further comprises one or more additional VHH specific for the E3 Ubiquitin Ligase, and/or one or more additional VHH specific for the target protein.

In frequent embodiments, the E3 Ubiquitin Ligase is a RING type E3 Ubiquitin Ligase. In certain embodiments, the E3 Ubiquitin Ligase is GRAIL, GODZILLA, and/or GOLIATH. Often the E3 Ubiquitin Ligase is one or more of RNF133, RNF148, RNF128, RNF149, RNF130, RNF150, RNF122, RNF43, ZNRF3, ZNRF4, RNF13, RNF167, AMFR, STVN1, RNF170, RNF121, RNF175, RNF139, RNF145, MARCHF5, VFPL1, RNFT1, RNF180, RNF103, RNF182, RNF5, RNF185, RNF19A, RNF19B, RNF144A, RNF144B, RNF217, MARCHF1, MARCHF8, MARCHF2, MARCHF3, MARCHF11, MARCHF4, MARCHF9, MARCHF6, NR1H4, RNF126, DCST1, RNF152, RNF186, CGRRF1, MUL1, TRIM13, TRIM59, or RNF112.

In often included embodiments, in the REULR constructs of formulas (a)-(e)^{A} is 2 or more, ^{B} is 2 or more, or wherein ^{A} is 2 or more and ^{B} is 2 or more. Also often, in the REULR constructs of formulas (a)-(e), ^{w} is 1 or more, ^{x} is 1 or more, ^{Y} is 1 or more, or ^{z} is 0, or 1 or more. Also often, in the REULR constructs of formulas (a)-(e), ^{w} is 0 or more, x is 2 or more, ^{Y} is 2 or more, or ^{z} is 0, or 1 or more. Also often, in the REULR constructs of formulas (a)-(e), ^{w} is 1 or more, x is 1 or more, ^{Y} is 1 or more, and z is 0, or 1 or more. Also often, in the REULR constructs of formulas (a)-(e), ^{w} is 0, ^{x} is 1 or more, ^{Y} is 1 or more, and z is 0, or 1 or more. Also often, in the REULR constructs of formulas (a)-(e), ^{A} is 3 or more, ^{B} is 3 or more, or wherein ^{A} is 3 or more and ^{B} is 3 or more.

In frequent embodiments, the target protein is a cell surface protein. Also frequently, the target protein is a receptor. In often included embodiments, the target protein is associated with a cell proliferative disorder or disease. Also often, the target protein is associated with one or more of a cancer, a neurological disorder, a lymphoma, a leukemia, diabetes, an autoimmune disorder, a viral infection, a bacterial infection, or a parasitic infection, Alzheimer's disease, or heart disease.

In further included embodiments, isolated nucleic acids encoding each of the REULR constructs contemplated herein are provided. In further included embodiments, vectors comprising isolated nucleic acids encoding each of the REULR constructs contemplated herein are provided, such vectors are often operably linked with an expression control sequence. In further included embodiments, cells comprising such vectors or isolated nucleic acids encoding each of the REULR constructs contemplated herein are provided.

Pharmaceutical compositions comprising the REULR constructs described herein are also provided, often including pharmaceutically acceptable carriers.

Also contemplated herein are kits comprising a REULR construct of the present disclosure. Such kits may also comprise a pharmaceutical composition, vector or host cell comprising the REULR construct.

Also provided herein are methods of modulating a target protein expressed on a cell, comprising contacting the cell expressing the target protein with a REULR construct comprising a variable domain heavy chain antibody (VHH) specific for a E3 Ubiquitin Ligase, and a VHH specific for target protein, and modulating the expression of the target protein. Often the modulation comprises degrading expression of the protein or converting the protein to a Ubiquitin substrate recruitment domain. Also provided herein are methods for treating a disease in a subject, comprising administering to the subject a therapeutically effective amount of a pharmaceutical composition comprising an exemplary REULR construct.

In such methods often the REULR construct is defined by the following formula:

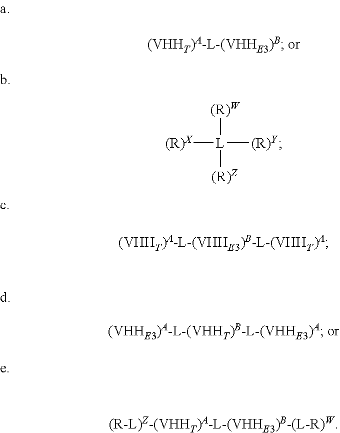

In such formulas defining exemplary REULR constructs, VHH is a variable domain heavy chain antibody specific for a target protein or an E3 Ubiquitin Ligase; R is VHH_{T} or VHH_{E3}; L is a linker molecule; T is a target protein such that the VHH is specific for the target protein; _{E3} is an E3 Ubiquitin Ligase such that the VHH is specific for the E3 Ubiquitin Ligase; ^{A} is 1 or more; ^{B} is 1 or more; w is 0, or 1 or more; ^{x} is 0, or 1 or more; ^{Y} is 0, or 1 or more; and z is 0, or 1 or more.

Also provided herein are linker molecules adapted to ligate the VHH specific for the E3 Ubiquitin Ligase to the VHH specific for a target protein in a manner such that the VHH specific for the E3 Ubiquitin Ligase and the VHH specific for a target protein are each independently available and able, respectively, to bind with the E3 Ubiquitin Ligase and the target protein.

In frequently included embodiments, the REULR construct, further comprises one or more additional VHH specific for the E3 Ubiquitin Ligase, and/or one or more additional VHH specific for the target protein.

In frequent embodiments, the E3 Ubiquitin Ligase is a RING type E3 Ubiquitin Ligase. In certain embodiments, the E3 Ubiquitin Ligase is GRAIL, GODZILLA, and/or GOLIATH Often the wherein the E3 Ubiquitin Ligase is one or more of RNF133, RNF148, RNF128, RNF149, RNF130, RNF150, RNF122, RNF43, ZNRF3, ZNRF4, RNF13, RNF167, AMFR, STVN1, RNF170, RNF121, RNF175, RNF139, RNF145, MARCHF5, VFPL1, RNFT1, RNF180, RNF103, RNF182, RNF5, RNF185, RNF19A, RNF19B, RNF144A, RNF144B, RNF217, MARCHF1, MARCHF8, MARCHF2, MARCHF3, MARCHF11, MARCHF4, MARCHF9, MARCHF6, NR1H4, RNF126, DCST1, RNF152, RNF186, CGRRF1, MUL1, TRIM13, TRIM59, or RNF112.

In often included embodiments, in the REULR constructs of formulas (a)-(e)^{A} is 2 or more, B is 2 or more, or wherein ^{A} is 2 or more and B is 2 or more. Also often, in the REULR constructs of formulas (a)-(e), ^{w} is 1 or more, x is 1 or more, ^{Y} is 1 or more, or ^{z} is 0, or 1 or more. Also often, in the REULR constructs of formulas (a)-(e), ^{w} is 0 or more, x is 2 or more, ^{Y} is 2 or more, or ^{z} is 0, or 1 or more. Also often, in the REULR constructs of formulas (a)-(e), ^{w} is 1 or more, x is 1 or more, ^{Y} is 1 or more, and z is 0, or 1 or more. Also often, in the REULR constructs of formulas (a)-(e), ^{w} is 0, ^{x} is 1 or more, ^{Y} is 1 or more, and z is 0, or 1 or more. Also often, in the REULR constructs of formulas (a)-(e), ^{A} is 3 or more, B is 3 or more, or wherein ^{A} is 3 or more and B is 3 or more.

In frequent embodiments, the target protein is a cell surface protein. Also frequently, the target protein is a receptor. In often included embodiments, the target protein is associated with a cell proliferative disorder or disease. Also often, the target protein is associated with one or more of a cancer, a neurological disorder, a lymphoma, a leukemia, diabetes, an autoimmune disorder, a viral infection, a bacterial infection, or a parasitic infection, Alzheimer's disease, or heart disease.

Also contemplated herein are methods of producing or manufacturing REULR constructs of the present disclosure by preparing the described REULR constructs, and compositions comprising such REULR constructs, using methods known in the art based on the teachings of the present disclosure.

All patents, patent applications, and publications mentioned herein, both supra and infra, are hereby incorporated by reference in their entireties.

### BRIEF DESCRIPTION OF THE FIGURES

The skilled person in the art will understand that the drawings, described below, are for illustration purposes only. The drawings are incorporated in and constitute a part of this specification.

FIGS. 1A & 1B depict a schematic of Receptor Elimination by E3 Ubiquitin Ligase Recruitment (FIG. 1A), and a schematic of Ligase Elimination by E3 Ubiquitin Ligase Recruitment (FIG. 1B).

FIGS. 2A & 2B depict representations and architectures within the transmembrane RNF (RING-Finger) E3 ubiquitin ligase family. FIG. 2A provides a schematic representation of the domain architecture of the transmembrane E3 Ubiquitin Ligase family members. FIG. 2B provides a representative domain architecture of a PA-TM-E3 Ligase including but not limited to RNF128 (GRAIL), RNF130 (GOLIATH), RNF167 (GODZILLA), RNF43 and ZNRF3 PA-TM-RING type E3 transmembrane ubiquitin ligases. Abbreviations: PA, protease-associated substrate recruitment domain; TM, putative transmembrane domain; RNF, RING Finger containing protein.

FIG. 3A depicts the expression pattern of transmembrane E3 Ligases in tissue (normalized RNAseq) and classification by family, number of transmembrane domains™ and sub cellular localization (SubCell) (A).

FIG. 3B depicts classification of transmembrane E3 Ligases by domain architecture.

FIG. 3C depicts classification of transmembrane E3 Ligases by number of transmembrane domains.

FIGS. 4A & 4B depict PD-1 receptor elimination by PD-1-GRAIL REULR with and without treatment of 3C Protease. FIG. 4A provides a schematic model of a Receptor PD1-GRAIL REULR concept. FIG. 4B depicts results of enforced recruitment of GRAIL to PD-1 using different version of PD-1-GRAIL REULR molecules, PD-1 cell surface levels are reduced when treated with intact versions of PD-1-GRAIL REULR molecules.

FIGS. 5A & 5B depict PD-1-GRAIL REULR with and without blocking of the E3 Ligase using excess of monomeric GRAIL VHH. FIG. 5A Provides a schematic model of a Receptor PD1-GRAIL REULR concept with and without pre-incubation of excess (40×) monomeric GRAIL VHH.

FIG. 5B Provides results of enforced recruitment of GRAIL to PD-1 using different version of PD-1-GRAIL REULR molecules, PD-1 cell surface levels are reduced when treated with intact versions of PD-1-GRAIL REULR molecules.

FIGS. 6A & 6B depict PD-1 receptor elimination by PD-1-GOLIATH REULR with and without treatment of 3C Protease. FIG. 6A Provides a schematic model of a Receptor PD1-GOLIATH REULR concept. FIG. 6B Provides results of enforced recruitment of GOLIATH to PD-1 using different version of PD-1-GOLIATH REULR molecules, PD-1 cell surface levels are reduced when treated with intact REULR version of PD-1-GOLIATH REULR molecules.

FIGS. 7A & 7B depict PD-1-GOLIATH REULR with and without blocking of the E3 Ligase using excess of monomeric GOLIATH VHH. FIG. 7A Provides a schematic model of a Receptor PD1-GOLIATH REULR concept with and without pre-incubation of excess (40×) monomeric GOLIATH VHH. FIG. 7B Provides results of enforced recruitment of GOLIATH to PD-1 using different version of PD-1-GOLIATH REULR molecules, PD-1 cell surface levels are reduced when treated with intact versions PD-1-GOLIATH REULR molecules.

FIGS. 8A-8C depict aspects of a PD1-GOLIATH REULR degradation pathway, specifically PD-1 degradation after treatment with PD-1-GOLIATH REULR in the presence or absence of proteasomal (MG132) or lysosomal (Bafilomycin) degradation pathway inhibitors. FIG. 8A, provides a schematic model of a Receptor PD1-GOLIATH REULR concept with and without treatment of 3C Protease. FIG. 8B is reproduced from Clague & Urbe, “Ubiquitin: Same Molecule, Different Degradation Pathways,” Cell 143(5): 682-85 (2010) and depicts ubiquitin dependent Receptor degradation pathways. FIG. 8C depicts results of an experiment to evaluate the PD-1 degradation pathway after treatment with PD1-GOLIATH REULR with and without 3C protease treatment in the presence or absence of MG132 or Bafilomycin small molecules.

FIGS. 9A & 9B depict EGFR receptor elimination by EGFR-GRAIL REULR. FIG. 9A provides a schematic model of a Receptor EGFR-GRAIL REULR concept. FIG. 9B provides results of enforced recruitment of GRAIL to EGFR using different version of EGFR-GRAIL REULR molecules, EGFR cell surface levels are reduced when treated with intact versions of EGFR-GRAIL REULR molecules.

FIGS. 10A & 10B depict EGFR receptor elimination by EGFR-GRAIL REULR. FIG. 10A provides a schematic model of a Receptor EGFR-GRAIL REULR concept. FIG. 10B provides results of enforced recruitment of GRAIL to EGFR using different version of EGFR-GRAIL, EGFR surface levels are reduced when treated with different versions of EGFR-GRAIL REULR molecules.

FIGS. 11A & 11B depict EGFR receptor elimination by EGFR-GOLIATH REULR with and without treatment of 3C Protease. FIG. 11A provides a schematic model of a Receptor EGFR-GOLIATH REULR concept. FIG. 11B depicts results of enforced recruitment of GOLIATH to EGFR using different version of EGFR-GOLIATH REULR molecules, EGFR receptor cell surface levels are reduced when treated with intact versions of GOLIATH-EGFR REULR molecules.

FIGS. 12A & 12B depict EpoR receptor elimination by EpoR-GRAIL REULR with and without treatment of 3C Protease. FIG. 12A provides a schematic model of a Receptor EpoR-GRAIL REULR concept. FIG. 12B depicts results of enforced recruitment of GRAIL to EpoR using a EpoR-GRAIL REULR molecule, EpoR receptor cell surface levels are reduced when treated with intact versions of a EpoR-GRAIL REULR molecule.

FIGS. 13A & 13B depict EpoR receptor elimination by EpoR-RNF43 REULR with and without treatment of 3C Protease. FIG. 13A provides a schematic model of a Receptor EpoR-RNF43 REULR concept. FIG. 13B depicts results of enforced recruitment of GRAIL to EpoR using a EpoR-RNF43 REULR molecule, EpoR receptor cell surface levels are reduced when treated with intact versions of a EpoR-RNF43 REULR molecule.

FIGS. 14A & 14B depict EpoR receptor elimination by EpoR-ZNRF3 REULR with and without treatment of 3C Protease. FIG. 14A provides a schematic model of a Receptor EpoR-ZNRF3 REULR concept. FIG. 14B depicts results of enforced recruitment of GRAIL to EpoR using a EpoR-ZNRF3 REULR molecule, EpoR receptor cell surface levels are reduced when treated with intact versions of a EpoR-ZNRF3 REULR molecule.

FIGS. 15A-15C depict GRAIL receptor elimination by GRAIL-GRAIL Fratricide REULR. FIG. 15A depicts a schematic model of a GRAIL-GRAIL Fratricide REULR concept. FIG. 15B depicts results of enforced recruitment of GRAIL to GRAIL by homodimerization and self-elimination using different version of GRAIL-GRAIL Fratricide REULR molecules with and without 3C Protease. FIG. 15C depicts results of enforced recruitment of GRAIL to GRAIL by homodimerization and self-elimination using different version of GRAIL-GRAIL Fratricide REULR molecules with and without pre-incubation of excess (40×) monomeric GRAIL VHH. GRAIL cell surface levels are reduced when treated with intact versions of GRAIL-GRAIL Fratricide REULR molecules.

FIGS. 16A & 16B depict RNF43 receptor elimination by RNF43-RNF43 Fratricide REULR. FIG. 16A depicts a schematic model of a RNF43-RNF43 Fratricide REULR concept. FIG. 16B depicts results of enforced recruitment of RNF43 to RNF43 by homodimerization and self-elimination using a RNF43-RNF43 Fratricide REULR molecules with and without pre-incubation of excess (40×) monomeric RNF43 VHH. RNF43 cell surface levels are reduced when treated with intact versions of RNF43-RNF43 Fratricide REULR molecules.

FIGS. 17A & 17B depict ZNRF3 receptor elimination by ZNRF3-ZNRF3 Fratricide REULR. FIG. 17A depicts a schematic model of a ZNRF3-ZNRF3 Fratricide REULR concept. FIG. 17B depicts results of enforced recruitment of ZNRF3 to ZNRF3 by homodimerization and self-elimination using a ZNRF3-ZNRF3 Fratricide REULR molecules with and without pre-incubation of excess (40×) monomeric ZNRF3 VHH. ZNRF3 cell surface levels are reduced when treated with intact versions of ZNRF3-ZNRF3 Fratricide REULR molecules.

FIGS. 18A & 18B depict RNF43 receptor elimination by RNF43-ZNRF3 Fratricide REULR. FIG. 18A depicts a schematic model of a RNF43-ZNRF3 Fratricide REULR concept. FIG. 18B depicts results of enforced recruitment of ZNRF3 to RNF43 by heterodimerization and elimination using a RNF43-ZNRF3 Fratricide REULR molecules with and without pre-incubation of excess (40×) monomeric RNF43/ZNRF3 VHH. RNF43 cell surface levels are reduced when treated with intact versions of RNF43-ZNRF3 Fratricide REULR molecules.

FIGS. 19A & 19B depict WNT Signaling potentiation assays by using different Fratricide REULR molecules. FIG. 19A depicts a schematic model of the Fratricide REULR concept. FIG. 19B depicts results of Fratricide REULR mediated potentiation of WNT Signaling using different configurations of Fratricide REULR molecules (RNF43-RNF43, ZNRF3-ZNRF3 or RNF43-ZNRF3) at various concentrations on HEK 293 Super Top Flash (STF) Wnt reporter cells. Activation and potentiation of the β-catenin dependent STF reporter only occurs when treated with intact version of the different ZNRF3 Fratricide REULR molecules.

FIGS. 20A-20F depict cell surface detection by flow cytometry of RNF128 (GRAIL), RNF130 (GOLIATH), RNF167 (GODZILLA), RNF43 and ZNRF3 on various human cell lines A431 (FIG. 20A), CaCo2 (FIG. 20B), HEK293T (FIG. 20C), HepG2 (FIG. 20D), UT-7 (FIG. 20E), and mouse Ba/F3 (FIG. 20F) cell lines by using streptavidin (APC-labelled) tetramerized GRAIL, GOLIATH, GODZILLA, RNF43 and ZNRF3 nanobodies (200 nM; titration series).

FIGS. 21A & 21B depict gating strategy for staining human PBMC using flow cytometric to identify T-Cells (CD4, CD8), Monocytes, NK and B-Cells. FIG. 21A depicts mRNA expression data for RNF128 (GRAIL and RNF130 (GOLIATH) reproduced form Uhlen et al., cited supra. In FIG. 21B PBMCs were either used unstimulated (NS), stimulated with CD3 or CD3+CD28 and subsequently used for cell surface staining using streptavidin (APC-labelled) tetramerized GRAIL and GOLIATH nanobodies.

FIGS. 22A-22F depict human PBMC nanobody staining results. Cells were stained with nanobodies to GRAIL and GOLIATH. FIG. 22A depicts CD4+ T-cells stained with a selection of antibody reagents. FIG. 22B depicts CD4+ T-cells stained with GRAIL nanobodies, in both blocked and unblocked format. FIG. 22C depicts CD4+ T-cells stained with GOLIATH nanobodies, in both blocked and unblocked format. FIG. 22D depicts CD8+ T-cells stained with a selection of antibody reagents. FIG. 22E depicts CD8+ T-cells stained with GRAIL nanobodies, in both blocked and unblocked format. FIG. 22F depicts CD8+ T-cells stained with GOLIATH nanobodies, in both blocked and unblocked format.

FIG. 23A-23I depict additional human PBMC nanobody staining results. Cells were stained with nanobodies to GRAIL and GOLIATH. FIG. 23A depicts B-cells stained with a selection of antibody reagents. FIG. 23B depicts B-cells stained with GRAIL nanobodies, in both blocked and unblocked format. FIG. 23C depicts B-cells stained with GOLIATH nanobodies, in both blocked and unblocked format. FIG. 23D depicts NK-cells stained with a selection of antibody reagents. FIG. 23E depicts NK-cells stained with GRAIL nanobodies, in both blocked and unblocked format. FIG. 23F depicts NK-cells stained with GOLIATH nanobodies, in both blocked and unblocked format.

FIG. 23G depicts monocytes stained with a selection of antibody reagents. FIG. 23H depicts monocytes stained with GRAIL nanobodies, in both blocked and unblocked format. FIG. 23I depicts B-cells stained with GOLIATH nanobodies, in both blocked and unblocked format.

FIGS. 24A-24D provide schematic depictions of exemplary REULR constructs of the present disclosure.

FIGS. 25A-25C depict an exemplary schematic of (FIG. 25A) an E3 ligase, including (FIG. 25B) a location of the signal peptide, PA domain, transmembrane domain, and RNF E3 ligase domain; and (FIG. 25C) exemplary nanobody sequences specific for the ECD of GRAIL, GOLIATH, and GODZILLA.

FIG. 26 depicts binding kinetics and binding affinity of the GRAIL, GOLIATH and GOLDZILLA nanobodies to the extracellular domains (ECD) of human GRAIL (RNF128), GOLIATH (RNF130) or GODZILLA (RNF167), which were assessed by Surface Plasmon Resonance (SPR) using a Biacore system. All nanobodies were purified by size exclusion (AKTA FPLC, GE Healthcare, Superdex 200 Increase; 280 nm absorbance). In addition, protein integrity and purity of the nanobodies and the ECDs of GRAIL (RNF128), GOLIATH (RNF1230) and GODZILLA (RNF167) were confirmed by SDS PAGE electrophoresis followed by standard Coomassie staining (data not shown).

FIG. 27 depicts measurement of titrations at equilibrium to determine K_{D}'s of the different nanobodies using Biacore Analysis Software (v.2.0.4, GE Healthcare) summarized in the following table.

FIGS. 28A-28C provide an exemplary schematic of (FIG. 28A) an E3 ligase, including (FIG. 28B) a location of the signal peptide, PA domain, transmembrane domain, and RNF E3 ligase domain; and (FIG. 28C) exemplary nanobody sequences specific for the ECD of RNF43.

FIG. 29 depicts binding kinetics and binding affinity of the RNF43 and ZNRF3 nanobodies to the extracellular domains (ECD) of human or mouse RNF43 and ZNRF3, which were assessed by Surface Plasmon Resonance (SPR) using a Biacore system. All nanobodies were purified by size exclusion (AKTA FPLC, GE Healthcare, Superdex 200 Increase; 280 nm absorbance). In addition, protein integrity and purity of the nanobodies and the ECDs of RNF43 and ZNRF3 were confirmed by SDS PAGE electrophoresis followed by standard Coomassie staining (data not shown).

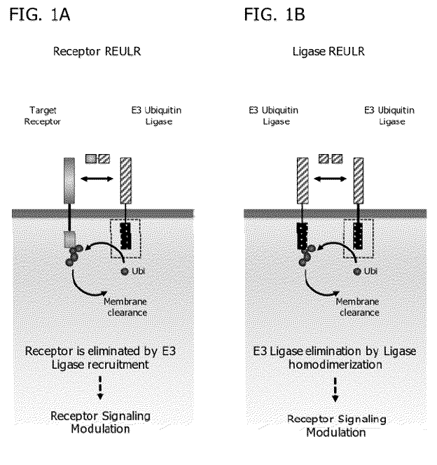

### DETAILED DESCRIPTION

For clarity of disclosure, and not by way of limitation, the detailed description of the various embodiments is provided into certain subsections that follow. A single embodiment may be discussed in multiple subsections.

Unless otherwise defined herein, scientific and technical terms used in connection with the present application shall have the meanings that are commonly understood by those of ordinary skill in the art to which this disclosure belongs. This disclosure is not limited to the particular methodology, protocols, and reagents, etc., described herein and as such can vary. The terminology used herein is for the purpose of describing particular embodiments only and is not intended to limit the scope of the present invention, which is defined solely by the claims. Definitions of common terms in immunology, and molecular biology can be found in The Merck Manual of Diagnosis and Therapy, 19th Edition, published by Merck Sharp & Dohme Corp., 2011 (ISBN 978-O-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, “a” or “an” means “at least one” or “one or more.”

As used herein, the term “and/or” may mean “and,” it may mean “or,” it may mean “exclusive-or,” it may mean “one,” it may mean “some, but not all,” it may mean “neither,” and/or it may mean “both.”

As used herein, “treatment” means any way the symptoms of a condition, disorder or disease are ameliorated or otherwise beneficially altered.

As used herein, “subject” often refers to an animal, including, but not limited to, a primate (e.g., human). The terms “subject” and “patient” are used interchangeably herein.

As used herein, a “modulation” refers to change or adjustment of presence and/or activity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters or by manual alignment and visual inspection. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The term “antibody” is used herein in the broadest sense in connection with monoclonal antibodies, polyclonal antibodies, monomers, dimers, multimers, multispecific antibodies (e.g., bispecific antibodies), heavy chain-only antibodies, three chain antibodies, TCAs, single chain Fv (scFv), nanobodies, etc., and also includes antibody fragments, so long as they exhibit the desired biological activity. Antibodies may be murine human, humanized, chimeric, or derived from other species. A functional or biologically active antibody or antigen-binding molecule (including heavy chain-only antibodies and multi-specific (e.g., bispecific) three-chain antibody-like molecules is one capable of exerting one or more of its natural activities in structural, regulatory, biochemical or biophysical events. For example, a functional antibody or other binding molecule, e.g., a VHH/nanobody, may have the ability to specifically bind an antigen and the binding may in turn elicit or alter a cellular or molecular event such as signal transduction or enzymatic activity. A functional antibody or other binding molecule, e.g., a VHH/nanobody, may also block ligand activation of a receptor or act as an agonist or antagonist.

The nanobodies/VHHs disclosed herein can be of any type (e.g., IgG, IgE, IgM, IgD, and IgA), class (e.g., IgG1, IgG2, IgG3, IgG4, IgA1 and IgA2) or subclass of immunoglobulin molecule, including engineered subclasses that provide for reduced or enhanced effector cell activity. The nanobodies/VHHs disclosed herein can be derived from any species. In one aspect, the nanobodies/VHHs disclosed herein are of largely human origin.

The terms “heavy chain-only antibody,” and “heavy chain antibody” are used interchangeably herein and refer, in the broadest sense, to antibodies, or more or more portions of an antibody, e.g., one or more arms of an antibody, lacking the light chain of a conventional antibody. The terms specifically include, without limitation, homodimeric antibodies comprising the VH antigen-binding domain and the CH2 and CH3 constant domains, in the absence of the CH1 domain; functional (antigen-binding) variants of such antibodies, soluble VH variants, Ig-NAR comprising a homodimer of one variable domain (V-NAR) and five C-like constant domains (C-NAR) and functional fragments thereof; and soluble single domain antibodies (sUniDabs™). The heavy chain-only antibody can be in the form of a dimer, in which two heavy chains are disulfide bonded or otherwise, covalently or non-covalently, attached with each other. The heavy chain-only antibody may belong to the IgG subclass, but antibodies belonging to other subclasses, such as IgM, IgA, IgD and IgE subclass, are also included herein. In a particular embodiment, a heavy chain antibody is of the IgG1, IgG2, IgG3, or IgG4 subtype, in particular the IgG1 subtype. In one embodiment, the heavy chain-only antibodies herein are used as a binding (targeting) domain of a chimeric antigen receptor (CAR). The definition specifically includes human heavy chain-only antibodies produced by human immunoglobulin transgenic rats (UniRat™), called UniAbs™. The variable regions (VH) of UniAbs™ are called UniDabs™, and are versatile building blocks that can be linked to Fc regions or serum albumin for the development of novel therapeutics with multi—specificity, increased potency and extended half-life. Since the homodimeric UniAbs™ lack a light chain and thus a VL domain, the antigen is recognized by one single domain, i.e., the variable domain of the heavy chain of a heavy —chain antibody (VH or VHH).

The terms “specific binding” or “specifically binding”, as used herein, in reference to the interaction of an antibody, a protein, or a peptide with a second chemical species, mean that the interaction is dependent upon the presence of a particular structure (e.g., an antigenic determinant or epitope) on the chemical species; for example, an antibody recognizes and binds to a specific protein structure rather than to proteins generally.

Ubiquitylation is a post-translational modification where ubiquitin is attached to a substrate protein. The addition of ubiquitin can affect proteins in many ways: It can signal for their degradation via the proteasomal or the ubiquitin dependent lysosomal degradation pathway, alter their cellular location, affect their activity, and promote or prevent protein interactions. Ubiquitylation is carried out in three main steps: activation, conjugation, and ligation, performed by ubiquitin-activating enzymes (E1s), ubiquitin-conjugating enzymes (E2s), and ubiquitin ligases (E3s), respectively. The result of this sequential cascade covalently attaches ubiquitin to lysine residues on the protein substrate via an isopeptide bond or to the amino group of the protein's N-terminus via a peptide bond.

Ubiquitin-activating enzyme (E1) starts the ubiquitylation process. The E1 enzyme along with ATP binds to the ubiquitin protein. The E1 enzyme then passes the ubiquitin protein to a second protein, called Ubiquitin carrier or conjugation protein (E2). The E2 protein complexes with a Ubiquitin protein ligase (E3), which mediates substrate recognition and catalyzes the covalent attachment of Ubiquitin to the substrate. In general, the ubiquitin ligase is involved in multi, oligo or poly-ubiquitylation with different chain linkages. Depending on the ubiquitin linkage and chain ubiquitylation marks a protein for degradation by the proteasome, the ubiquitin dependent lysosomal degradation pathway or other ubiquitin dependent signaling transduction. While cytosolic E3 ligases mediate substrate recognition via specific domains or utilize E2 enzymes in multisubunit complexes (e.g., Cullins), transmembrane E3 ligases operate differently. For example, they are present in various organelles and play a role in a variety of cellular and organelle functions, such as protein quality control and trafficking, apoptosis, cell proliferation and differentiation, mitochondrial dynamics, immune regulation, and signaling.

Assays for ubiquitin ligation cascade proteins and inhibitors, activators and modulators thereof include, e.g., expressing ubiquitin ligation cascade protein in vitro, in cells, or cell membranes, applying putative modulator compounds, and then determining the effects on activity. Assays for activity of ubiquitin ligation cascade proteins are known in the art.

The present disclosure concerns receptor modulation by E3 ubiquitin ligase recruitment (REULR), which is a technology platform using REULR constructs defined herein that uses bispecific or multi-modular nanobody/antibody molecules, for example VHH1, VHH2, VHH3, etc., to ligate cell surface targets of interest to cell surface E3 Ub ligases. This causes degradation of the target protein and membrane clearance, and loss of its activity in essentially a protein knockdown process. As a technology platform, REULR is distinct from classical antagonism of a cell surface protein because it makes, reduces, or eliminates the presence of the target protein on the cell surface. Therefore, REULR constructs are often adapted to modulate or eliminate target cell surface proteins and/or tune a therapeutic effect. This is very different from classical antagonism, which has an all or none effect.

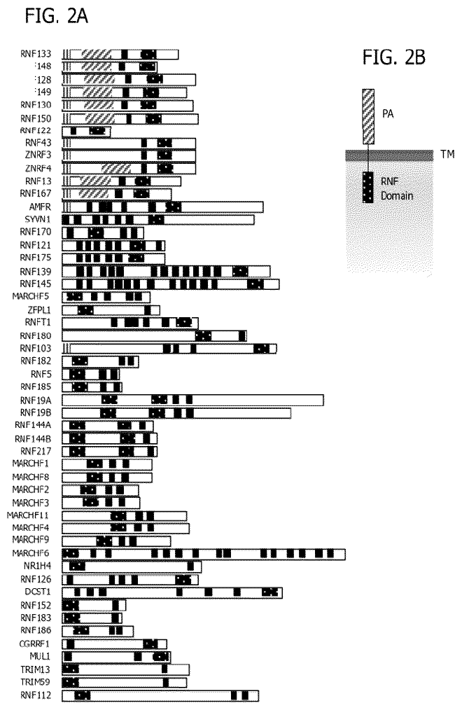

There are about 60 different transmembrane E3 Ub ligases, each of which has the potential to be redirected to cell surface targets to initiate destruction. FIG. 2 depicts members of the transmembrane RNF (RING-Finger) E3 ubiquitin ligase family with a schematic representation of™-RING type transmembrane E3 Ubiquitin Ligases are provided in FIG. 2A. The PA-TM-RING family is generally defined by three conserved domains, the protease-associated (PA) domain acting as a substrate recruitment domain, the transmembrane domain and the catalytic ubiquitin ligase E3 RING-H2 finger domain (RNF). FIG. 2B depicts the domain architecture of RNF128 (GRAIL), RNF130 (GOLIATH), RNF167 (GODZILLA), RNF43 and ZNRF3 PA-TM-RING type E3 transmembrane ubiquitin ligase domain containing proteins. Signal peptide, PA domain, transmembrane domain and RNF E3 ligase domains are depicted. FIG. 2C depicts a comparison of the domain architecture of putative human transmembrane RNF E3 ligase domain containing proteins. RNF133, RNF148, RNF128, RNF149, RNF130, RNF150, RNF122, RNF43, ZNRF3, ZNRF4, RNF13, RNF167, AMFR, STVN1, RNF170, RNF121, RNF175, RNF139, RNF145, MARCHF5, VFPL1, RNFT1, RNF180, RNF103, RNF182, RNF5, RNF185, RNF19A, RNF19B, RNF144A, RNF144B, RNF217, MARCHF1, MARCHF8, MARCHF2, MARCHF3, MARCHF11, MARCHF4, MARCHF9, MARCHF6, NR1H4, RNF126, DCST1, RNF152, RNF186, CGRRF1, MUL1, TRIM13, TRIM59, and RNF112 are exemplary E3 ubiquitin ligases that are contemplated as targets for the REULR constructs described herein.

The following Table A sets forth E3 ubiquitin ligases that may be targeted by REULR constructs of the present disclosure.

TABLE A

| Gene | Trans. | | |
|---|---|---|---|
| Name | Domain | Protein Name | Family |
| RNF133 | TM1 | E3 ubiquitin-protein ligase RNF133 | PA-TM-RING |
| RNF148 | TM1 | E3 ubiquitin-protein ligase RNF148 | PA-TM-RING |
| RNF128 | TM1 | E3 ubiquitin-protein ligase RNF128 | PA-TM-RING |
| RNF149 | TM1 | E3 ubiquitin-protein ligase RNF149 | PA-TM-RING |
| RNF130 | TM1 | E3 ubiquitin-protein ligase RNF130 | PA-TM-RING |
| RNF150 | TM1 | RING finger protein 150 | PA-TM-RING |
| RNF122 | TM1 | RING finger protein 122 | RING |
| ZNRF3 | TM1 | E3 ubiquitin-protein ligase ZNRF3 | PA-TM-RING |
| ZNRF4 | TM1 | E3 ubiquitin-protein ligase ZNRF4 | RING |
| RNF43 | TM1 | E3 ubiquitin-protein ligase RNF43 | PA-TM-RING |
| RNF13 | TM1 | E3 ubiquitin-protein ligase RNF13 | PA-TM-RING |
| RNF167 | TM1 | E3 ubiquitin-protein ligase RNF167 | PA-TM-RING |
| AMFR | TM5 | E3 ubiquitin-protein ligase AMFR | RING |
| SYVN1 | TM6 | E3 ubiquitin-protein ligase synoviolin | RING |
| RNF170 | TM3 | E3 ubiquitin-protein ligase RNF170 (Putative | RING |
| | | LAG1-interacting protein) | |
| RNF121 | TM6 | RING finger protein 121 | RING |
| RNF175 | TM5 | RING finger protein 175 | RING |
| RNF139 | TM12 | E3 ubiquitin-protein ligase RNF139 | RING |
| RNF145 | TM12 | RING finger protein 145 | RING |
| MARCHF5 | TM4 | E3 ubiquitin-protein ligase MARCHF5 | MARCH |
| | | (Membrane-associated RING finger protein 5) | |
| ZFPL1 | TM1 | Zinc finger protein-like 1 (Zinc finger protein | RING |
| | | MCG4) | |
| RNFT1 | TM5 | E3 ubiquitin-protein ligase RNFT1 (RING finger | RING |
| | | and transmembrane domain-containing protein 1) | |
| RNF180 | TM1 | E3 ubiquitin-protein ligase RNF180 | RING |
| RNF103 | TM3 | E3 ubiquitin-protein ligase RNF103 | RING |
| RNF182 | TM2 | E3 ubiquitin-protein ligase RNF182 | RING |
| RNF5 | TM2 | E3 ubiquitin-protein ligase RNF5 | RING |
| RNF185 | TM2 | E3 ubiquitin-protein ligase RNF185 | RING |
| RNF19A | TM2 | E3 ubiquitin-protein ligase RNF19A (Double | RBR |
| | | ring-finger protein) | |
| RNF19B | TM2 | E3 ubiquitin-protein ligase RNF19B (Natural | RBR |
| | | killer lytic-associated molecule) | |
| RNF144A | TM1 | E3 ubiquitin-protein ligase RNF144A | RBR |
| RNF144B | TM1 | E3 ubiquitin-protein ligase RNF144B (IBR | RBR |
| | | domain-containing protein 2) | |
| RNF217 | TM1 | Probable E3 ubiquitin-protein ligase RNF217 | RBR |
| | | (IBR domain-containing protein 1) | |
| MARCHF1 | TM2 | E3 ubiquitin-protein ligase MARCHF1 | MARCH |
| | | (Membrane-associated RING finger protein 1) | |
| MARCHF8 | TM2 | E3 ubiquitin-protein ligase MARCHF8 (Cellular | MARCH |
| | | modulator of immune recognition; Membrane- | |
| | | associated RING finger protein 8) | |
| MARCHF2 | TM2 | E3 ubiquitin-protein ligase MARCHF2 | MARCH |
| | | (Membrane-associated RING finger protein 2) | |
| MARCHF3 | TM2 | E3 ubiquitin-protein ligase MARCHF3 | MARCH |
| | | (Membrane-associated RING finger protein 3) | |
| MARCHF11 | TM2 | E3 ubiquitin-protein ligase MARCHF11 | MARCH |
| | | (Membrane-associated RING finger protein 11) | |
| MARCHF4 | TM2 | E3 ubiquitin-protein ligase MARCHF4 | MARCH |
| | | (Membrane-associated RING finger protein 4) | |
| MARCHF9 | TM2 | E3 ubiquitin-protein ligase MARCHF9 | MARCH |
| | | (Membrane-associated RING finger protein 9) | |
| MARCHF6 | TM14 | E3 ubiquitin-protein ligase MARCHF6 (Doa10 | MARCH |
| | | homolog; Membrane-associated RING finger | |
| | | protein 6) | |
| NR1H4 | TM1 | Bile acid receptor (Farnesoid X-activated | RING |
| | | receptor) | |
| RNF126 | TM4 | E3 ubiquitin-protein ligase RNF126 | RING |
| DCST1 | TM6 | E3 ubiquitin-protein ligase DCST1 | RING |
| RNF152 | TM1 | E3 ubiquitin-protein ligase RNF152 | RING |
| RNF183 | TM1 | E3 ubiquitin-protein ligase RNF183 | RING |
| RNF186 | TM2 | E3 ubiquitin-protein ligase RNF186 | RING |
| CGRRF1 | TM1 | Cell growth regulator with RING finger domain | RING |
| | | protein 1 | |
| MUL1 | TM2 | RING finger protein 218 | RING |
| TRIM13 | TM1 | E3 ubiquitin-protein ligase TRIM13 (B-cell | TRIM |
| | | chronic lymphocytic leukemia tumor suppressor | |
| | | Leu5) | |
| TRIM59 | TM1 | Tripartite motif-containing protein 59 | TRIM |
| RNF112 | TM2 | RING finger protein 112 (Brain finger protein) | RING |

For example, in often included embodiments, a REULR construct comprises a VHH specific for the transmembrane domain of any one or more of the following E3 ubiquitin ligases and a VHH specific for one or more target proteins. Often such a REULR construct comprises a linker molecule to ligate the two or more VHHs. Also for example, in often included embodiments, a REULR construct comprises a VHH specific for the transmembrane domain of a E3 ubiquitin ligase PA-TM-RING family member and a VHH specific for one or more target proteins. Also for example, in often included embodiments, a REULR construct comprises a VHH specific for the transmembrane domain of a E3 ubiquitin ligase RING family member and a VHH specific for one or more target proteins. Also for example, in often included embodiments, a REULR construct comprises a VHH specific for the transmembrane domain of a E3 ubiquitin ligase MARCH family member and a VHH specific for one or more target proteins. Also for example, in often included embodiments, a REULR construct comprises a VHH specific for the transmembrane domain of a E3 ubiquitin ligase RBR family member and a VHH specific for one or more target proteins. Also for example, in often included embodiments, a REULR construct comprises a VHH specific for the transmembrane domain of a E3 ubiquitin ligase TRIM family member and a VHH specific for one or more target proteins.

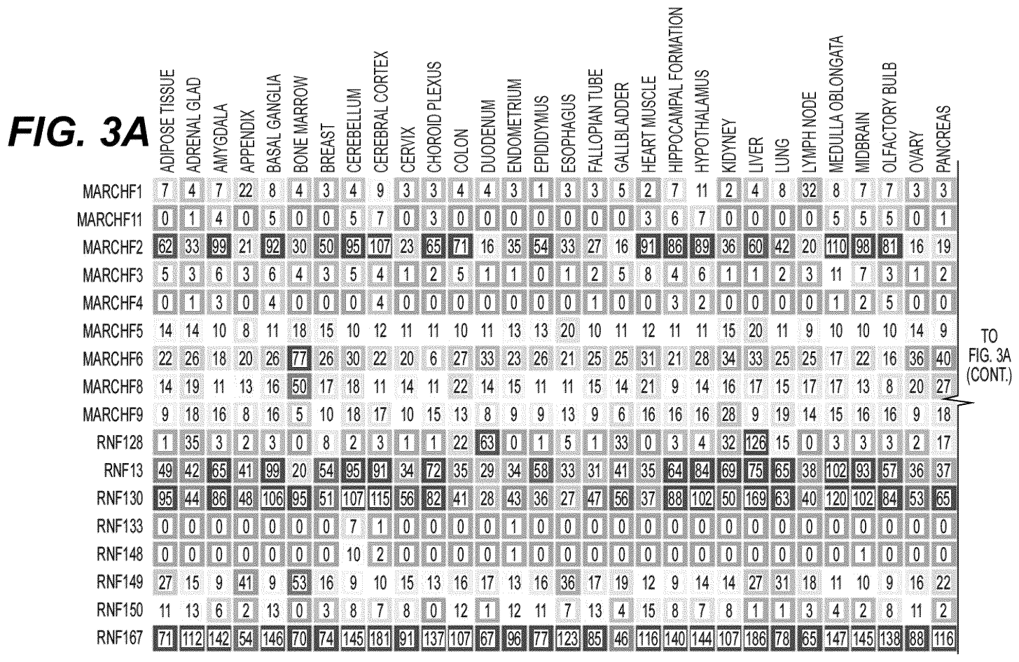

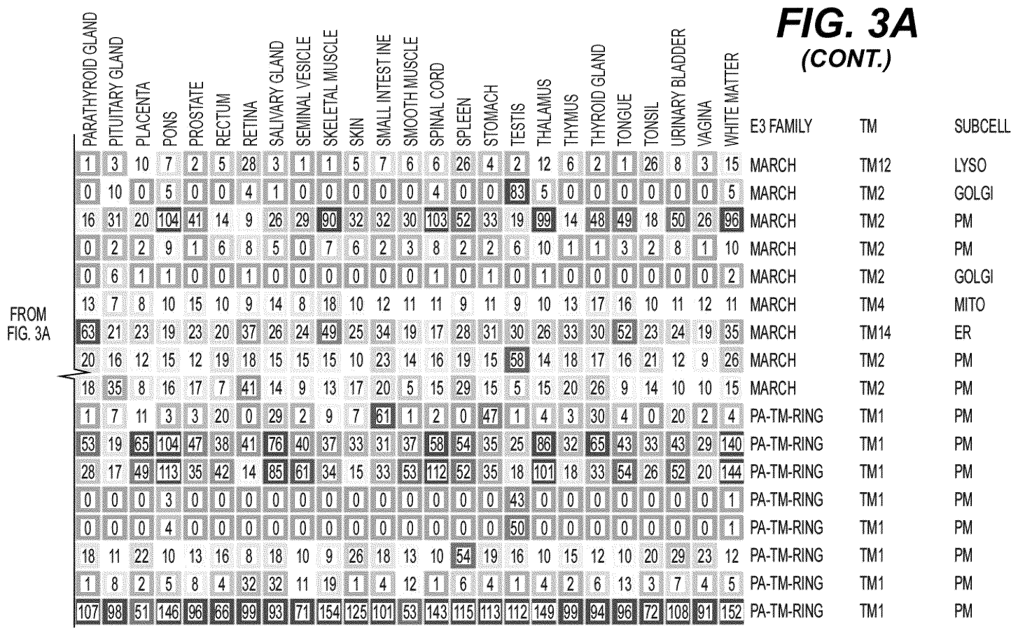

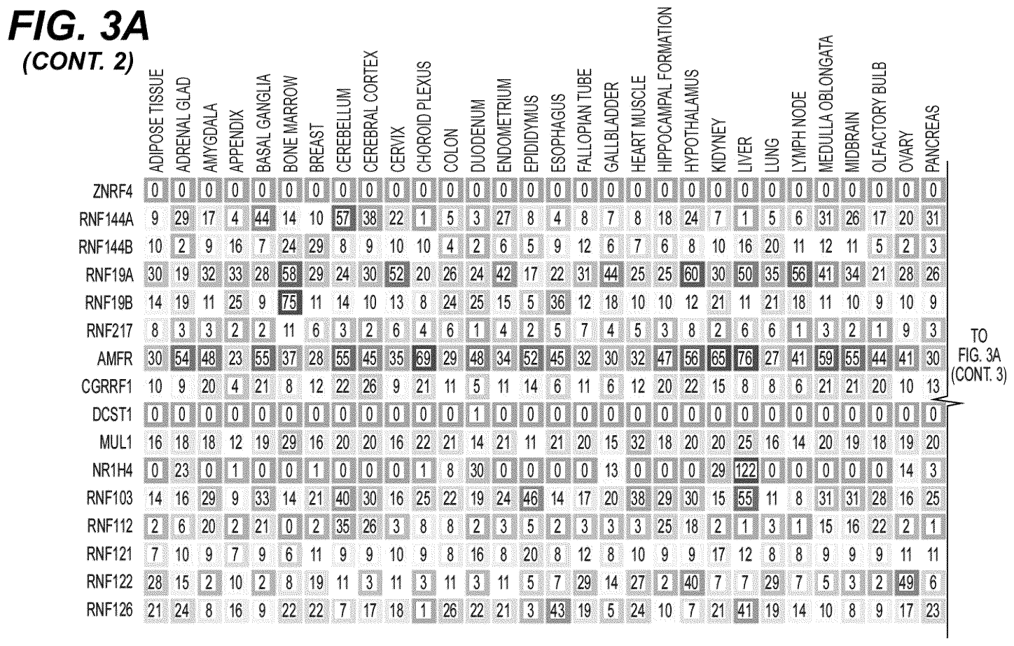

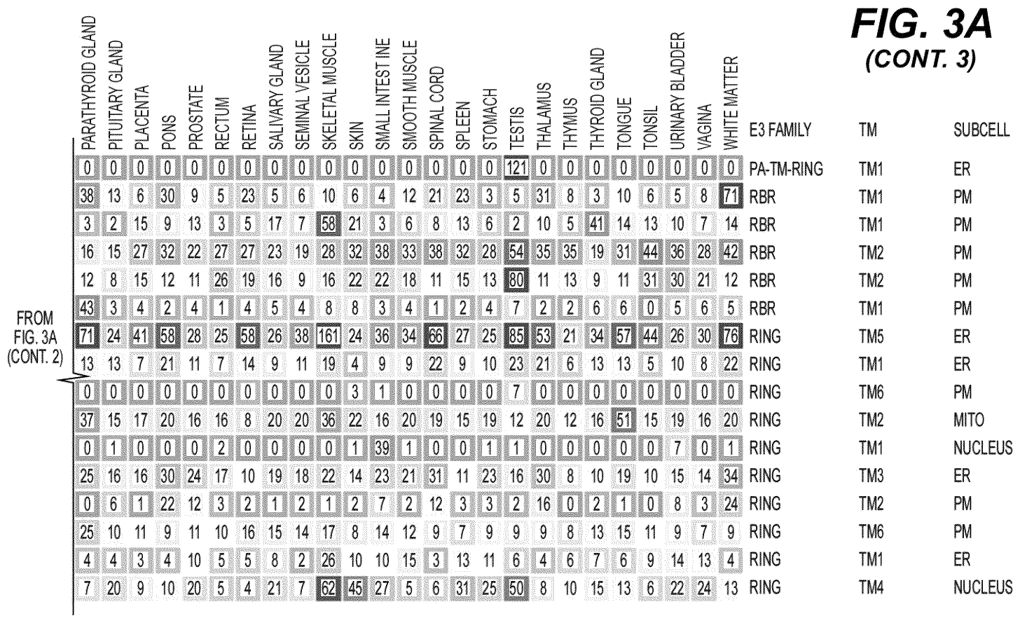

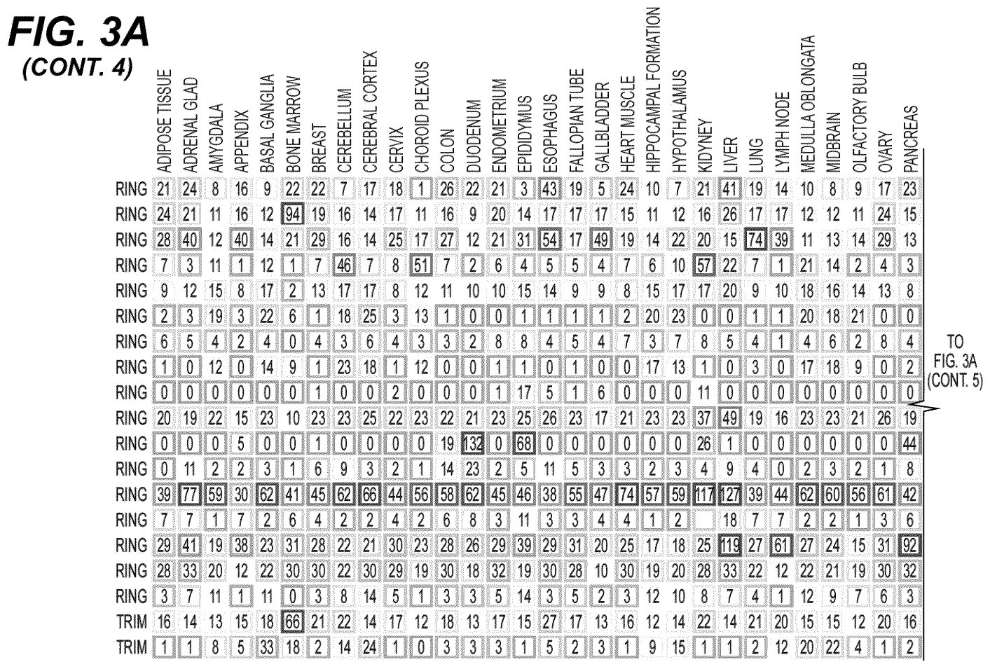

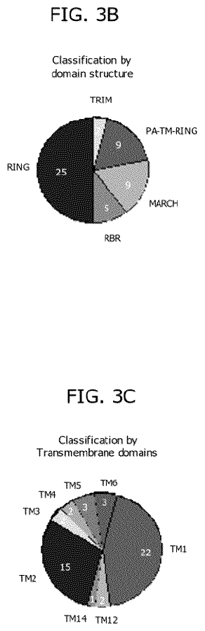

As shown in FIG. 3 (A-C), transmembrane E3 Ligases are expressed in a host of tissues. With specificity noted herein for these specific REULR constructs and others contemplated herein, these tissues and cell types can be targeted for REULR construct use in, for example, therapeutic or prophylactic use. For example, exemplary REULR constructs may be prepared to target specific transmembrane proteins having a ubiquitin acceptor site in their intracellular domain in these tissues and cells where one or more of GRAIL, GOLIATH, GODZILLA, RNF43 and/or ZNRF3 are expressed. Moreover, exemplary REULR constructs may be prepared to target specific transmembrane proteins having a ubiquitin acceptor site in their intracellular domain in these tissues and cells where one or more of RNF133, RNF148, RNF128, RNF149, RNF130, RNF150, RNF122, RNF43, ZNRF3, ZNRF4, RNF13, RNF167, AMFR, STVN1, RNF170, RNF121, RNF175, RNF139, RNF145, MARCHF5, VFPL1, RNFT1, RNF180, RNF103, RNF182, RNF5, RNF185, RNF19A, RNF19B, RNF144A, RNF144B, RNF217, MARCHF1, MARCHF8, MARCHF2, MARCHF3, MARCHF11, MARCHF4, MARCHF9, MARCHF6, NR1H4, RNF126, DCST1, RNF152, RNF186, CGRRF1, MUL1, TRIM13, TRIM59, and/or RNF112 are expressed.

In the present disclosure REULR constructs comprised of VHHs to the mouse and human E3 ligases GRAIL, GODZILLA, GOLIATH, RNF43 and ZNRF3 are provided. It is contemplated that in view of the presently provided methods and data, that VHHs to the known mouse and human E3 ligases of FIG. 2C are also prepared using known methods. These REULR constructs also contain VHHs specific for a selection of target proteins. While target proteins PD-1, EGFR, EPOR and GPCR are described and exemplified, these examples are intended to be non-limiting and merely illustrative of additional contemplated REULR constructs and their uses with these and other target proteins as described herein. For example, it is contemplated that an exemplary target protein includes any transmembrane receptor with a ubiquitin acceptor site in its intracellular domain. In the present context the herein described REULR constructs effectively achieve gene silencing without gene editing since the focus is on transmembrane proteins.

The Erythropoietin (EPO) receptor is one such exemplary target protein. According to the present disclosure a REULR construct includes a VHH or scFV to the EPO receptor linked to a VHH specific for one or more of GRAIL, GODZILLA, GOLIATH, RNF43 and/or ZNRF3. According to the present disclosure a REULR construct includes a VHH or scFV to the EPO receptor linked to a VHH specific for one or more of RNF133, RNF148, RNF128, RNF149, RNF130, RNF150, RNF122, RNF43, ZNRF3, ZNRF4, RNF13, RNF167, AMFR, STVN1, RNF170, RNF121, RNF175, RNF139, RNF145, MARCHF5, VFPL1, RNFT1, RNF180, RNF103, RNF182, RNF5, RNF185, RNF19A, RNF19B, RNF144A, RNF144B, RNF217, MARCHF1, MARCHF8, MARCHF2, MARCHF3, MARCHF11, MARCHF4, MARCHF9, MARCHF6, NR1H4, RNF126, DCST1, RNF152, RNF186, CGRRF1, MUL1, TRIM13, TRIM59, and/or RNF112. Such exemplary REULR constructs would cause an elimination or modulation in the EPO receptor. While not intending to be bound by any specific theory of operation, this would inhibit uncontrolled proliferation of cancer cells that carry the EPO receptor in myloproliferative disorders. Similar targeting can be achieved with any of a variety of other target proteins in cells expressing E3 Ub ligases.

An exemplary REULR construct may contain a VHH to these exemplary target proteins or other target proteins.

Herein presented are functional activities of REULR proteins GRAIL, GOLIATH, GODZILLA, RNF43 and ZNRF3 in the elimination of target proteins from cell surfaces.

The target protein of the presently described REULR constructs is often a cell surface receptor, transporter, or channel that contains a ubiquitin acceptor site in its intracellular domain a cell that also expresses an E3 Ub ligase. The target protein is also often a receptor that is or is associated with (as the case may be) a disease or disorder, oncogenic potential, an immune checkpoint, an innate/adaptive immunity, HIV, inflammation, autoimmunity, a tumor associated antigen, a tumor antigen, or other known disease-associated proteins, including other receptors, that contain a ubiquitin acceptor site in their intracellular domain. As described herein, a target cell contains or expresses the target protein.

Moreover methods of treating a disease associated with a target protein are contemplated, which methods involve administering to a subject in need thereof a therapeutically effective amount of a pharmaceutical composition comprising a REULR construct specific for the target protein. In some embodiments, a REULR construct can be used to treat cell proliferative diseases, including cancer, which involve the unregulated and/or inappropriate proliferation of cells, sometimes accompanied by destruction of adjacent tissue and growth of new blood vessels, which can allow invasion of cancer cells into new areas, i.e., metastasis. Included within conditions treatable with a REULR construct are non-malignant conditions that involve inappropriate cell growth, including colorectal polyps, cerebral ischemia, gross cystic disease, polycystic kidney disease, benign prostatic hyperplasia, and endometriosis. A REULR construct can be used to treat a hematologic or solid tumor malignancy. More specifically, cell proliferative diseases that can be treated using a REULR construct are, for example, cancers including mesotheliomas, squamous cell carcinomas, myelomas, osteosarcomas, glioblastomas, gliomas, carcinomas, adenocarcinomas, melanomas, sarcomas, acute and chronic leukemias, lymphomas, and meningiomas, Flodgkin's disease, Sezary syndrome, multiple myeloma, and lung, non-small cell lung, small cell lung, laryngeal, breast, head and neck, bladder, ovarian, skin, prostate, cervical, vaginal, gastric, renal cell, kidney, pancreatic, colorectal, endometrial, and esophageal, hepatobiliary, bone, skin, and hematologic cancers, as well as cancers of the nasal cavity and paranasal sinuses, the nasopharynx, the oral cavity, the oropharynx, the larynx, the hypolarynx, the salivary glands, the mediastinum, the stomach, the small intestine, the colon, the rectum and anal region, the ureter, the urethra, the penis, the testis, the vulva, the endocrine system, the central nervous system, and plasma cells.

In some embodiments, a REULR construct can be administered concurrently with, before, or after a variety of drugs and treatments widely employed in cancer treatment such as, for example, chemotherapeutic agents, non-chemotherapeutic, anti-neoplastic agents, and/or radiation. For example, chemotherapy and/or radiation can occur before, during, and/or after any of the treatments described herein. Examples of chemotherapeutic agents are discussed herein and include, but are not limited to, cisplatin, taxol, etoposide, mitoxantrone (Novantrone*), actinomycin D, cycloheximide, camptothecin (or water-soluble derivatives thereof), methotrexate, mitomycin (e.g., mitomycin C), dacarbazine (DTIC), anti-neoplastic antibiotics such as adriamycin (doxorubicin) and daunomycin, and all the chemotherapeutic agents mentioned herein.

A REULR construct can also be used to treat infectious disease, for example a chronic hepatis B virus (HBV) infection, a hepatis C virus (HCV) infection, a human immunodeficiency virus (HIV) infection, an Epstein-Barr virus (EBV) infection, or a cytomegalovirus (CMV) infection, among many others.

A REULR construct can find further use in other kinds of conditions where it is beneficial to deplete certain cell types. For example, depletion of human eosinophils in asthma, excess human B cells in systemic lupus erythematosus, excess human Th2 T cells in autoimmune conditions, or pathogen-infected cells in infectious diseases can be beneficial. In a fibrotic condition, it can be useful to deplete cells forming fibrotic tissue.

Therapeutically effective doses of a REULR construct can be administered. The amount of REULR construct that constitutes a therapeutically dose may vary with the indication treated, the weight of the patient, the calculated skin surface area of the patient. Dosing of a REULR construct can be adjusted to achieve the desired effects. In many cases, repeated dosing may be required.

A REULR construct, or a pharmaceutical composition containing a REULR construct, can be administered by any feasible method. Protein therapeutics are often ordinarily be administered by a parenteral route, for example by injection. Without proper protective and release mechanisms in a special pharmaceutical formulation, dosing or administration protocol, proteins administered orally can be hydrolyzed in the acid environment of the stomach. Subcutaneous, intramuscular, intravenous, intraarterial, intralesional, or peritoneal bolus injections are optional routes of administration. A REULR construct can also be administered via infusion, for example intravenous or subcutaneous infusion. Topical administration is also contemplated, for example, in connecting with treating diseases involving the skin. Similarly, a REULR construct can be administered through mucus membranes, for example by intra-nasal, sublingual, vaginal, or rectal administration or administration as an inhalant. Further, certain appropriate pharmaceutical compositions comprising a REULR construct can be administered orally. Delivery of a REULR construct by inhalation is also contemplated, for example, nasal or oral inhalation, use of a nebulizer, inhalation of the bispecific binding construct in aerosol form, and the like.

Treatment encompasses alleviation of at least one symptom or other embodiment of a disorder, or reduction of disease severity, and the like. A REULR construct according to the present invention need not effect a complete cure, or eradicate every symptom or manifestation of a disease, to constitute a viable therapeutic agent. As is recognized in the pertinent field, drugs employed as therapeutic agents may reduce the severity of a given disease state but need not abolish every manifestation of the disease to be regarded as useful therapeutic agents. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. One embodiment of the invention is directed to a method comprising administering to a patient a REULR construct of the disclosure in an amount and for a time sufficient to induce a sustained improvement over baseline of an indicator that reflects the severity of the disorder.

Preventative or prophylactic use encompasses prevention of at least one symptom or other aspect of a disorder or side effect. A prophylactically administered treatment incorporating a REULR construct according to the present disclosure need not be completely effective in preventing the onset of a condition to constitute a viable prophylactic agent. Simply reducing the likelihood that the disease will occur or worsen in a subject, is sufficient.

Effective doses of REULR construct compositions for the treatment of disease vary depending upon a variety of factors, including route of administration, target site, physiological state of the patient, whether the patient is human or an animal, co-administered medications, and/or whether treatment is prophylactic or therapeutic. Usually, the patient is a human, but nonhuman mammals may also be treated, e.g., companion animals such as dogs, cats, horses, etc., laboratory mammals such as rabbits, mice, rats, etc., and the like. Treatment dosages are often titrated to optimize safety and efficacy. Dosage levels can be readily determined by an ordinarily skilled clinician, and can be modified as required, e.g., as required to modify a response to therapy. The amount of REULR construct that can be included to produce a single dosage form varies depending upon the host treated and the particular mode of administration. Dosage unit forms generally contain between from about 1 mg to about 500 mg of a REULR construct. In some embodiments, the therapeutic dosage the REULR construct may range from about 0.0001 to 100 mg/kg, and more usually 0.01 to 5 mg/kg, of the subject's body weight. For example, dosages of the REULR construct can be 1 mg/kg body weight or 10 mg/kg body weight or within the range of 1-10 mg/kg. One exemplary treatment regime entails administration once every week, every two weeks, every three weeks, once a month, bimonthly every three months, every six months, etc. Intervals can also be irregular as indicated by measuring blood levels of the REULR construct in the patient. Alternatively, RELULR construct of the present invention can be administered as a sustained release formulation, in which case less frequent administration is required. Dosage and frequency vary depending on the half-life of the REULR construct in the subject.

In certain embodiments, therapeutic compositions are prepared as injectables, either as liquid solutions or suspensions; solid forms suitable for solution in, or suspension in, liquid vehicles prior to injection can also be prepared. The presently contemplated pharmaceutical compositions are suitable for intravenous or subcutaneous administration, directly or after reconstitution of solid (e.g., lyophilized) compositions. The preparation also can be emulsified or encapsulated in liposomes or micro particles such as polylactide, polyglycolide, or copolymer for enhanced adjuvant effect, as discussed above. The REULR constructs of the present disclosure can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the REULR construct described herein can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays, and animal studies can be used in formulating a dosage range of REULR constructs that is not toxic for use in humans. The dosage of the REULR constructs described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

The compositions for administration will commonly comprise a REULR construct dissolved in a pharmaceutically acceptable carrier, preferably an aqueous carrier. A variety of aqueous carriers can be used, e.g., buffered saline and the like. These solutions are sterile and generally free of undesirable matter. These REULR construct compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of REULR construct in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

Also within the scope of the invention are kits comprising the REULR constructs and formulations thereof, of the disclosure and instructions for use. The kit can further contain a least one additional reagent, e.g., a codrug such as a chemotherapeutic agent or other therapeutic or palliative agent, etc. Kits typically include a label indicating the intended use of the contents of the kit. The term label includes any writing, or recorded material supplied on or with a kit, or which otherwise accompanies a kit.

While not intending to be limited, some options of disease that can be treated include neurological disorders, cancer, lymphoma, leukemia, diabetes, autoimmune disorders, viral infections, bacterial infections, parasitic infections, Alzheimer's disease, and/or heart disease.

The described pharmaceutical compositions comprising a REULR construct may be administered in a manner suitable to transmit the REULR construct in the composition to the target protein based on its location of expression. Injection (intravenous, intramuscular, intraperitoneal, subcutaneous), oral, sublingual, oralmucosal, transdernal, ophthalmic, inhaled, vaginal, rectal, intranasal, topical, etc.

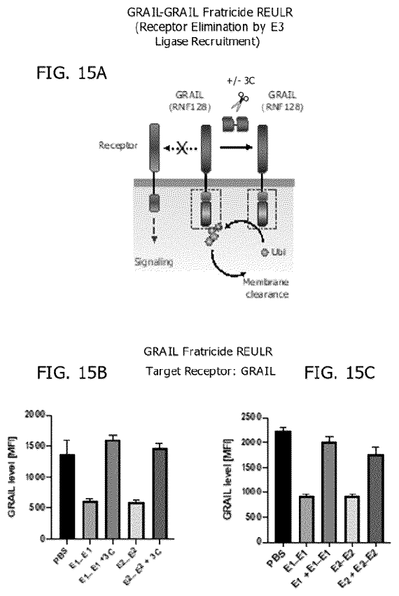

Also herein presented in FIGS. 15-19 are functional activities of REULR constructs (GRAIL, RNF43, ZNRF3) that degrade “self-degrade” through the use of the presently described REULR constructs. This describes the homo —or heterodimeization of two E3 ligases e.g., GRAIL-GRAIL, RNF43-RNF43, ZNRF3-ZNRF3 or RNF43-ZNRF3 are herein termed Fratricide REULR molecules. While not intending to be limited to a theory of operation, in these cases the E3 Ligase itself becomes the target, e.g., GRAIL, (Gene Related to Anergy In Lymphocytes) is upregulated upon T cell anergy and may serve to limit autoreactive T cell responses and inhibit cytokine gene transcription. It is found that modulating GRAIL, for example, is beneficial for modulating T-Cell activation.

RNF43 and ZNRF3 are essential modulator of the WNT signaling pathway by negatively mediating the regulation of FZD receptors. In addition, RNF43 and ZNRF3 are commonly altered in serrated pathway colorectal tumorigenesis (CRCs) and data suggest that RNF43 mutations cooperate with KRAS mutations to promote multi-step tumorigenesis via the Wnt-Ras-p53 axis in human colon cancers.

Also herein presented are cell surface expression levels of REULR proteins GRAIL, GOLIATH, GODZILLA, RNF43 and ZNRF3 in various human and mouse cell lines.

Also herein presented are cell surface expression levels of REULR proteins GRAIL and GODZILLA in blood cells using the E3 Ub ligase VHH as a staining reagent for fluorescence activated cell sorting (FACS) analysis.

Furthermore, a variety of exemplary REULR construct amino acid sequences are provided, along with functional data that proves the activity of the identified REULR constructs. Based on these examples and information concerning their production and use, generalizations made and claimed herein are proper for the genus encompassing these exemplified REULR constructs.

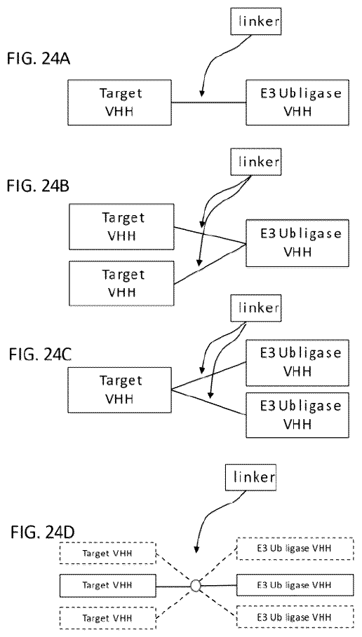

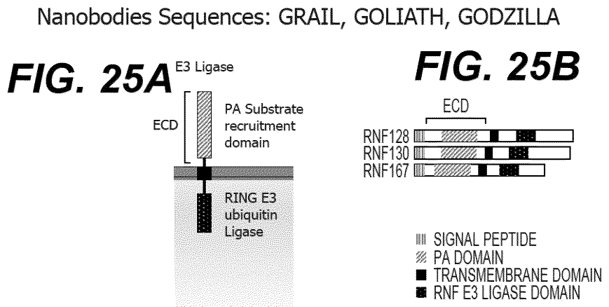

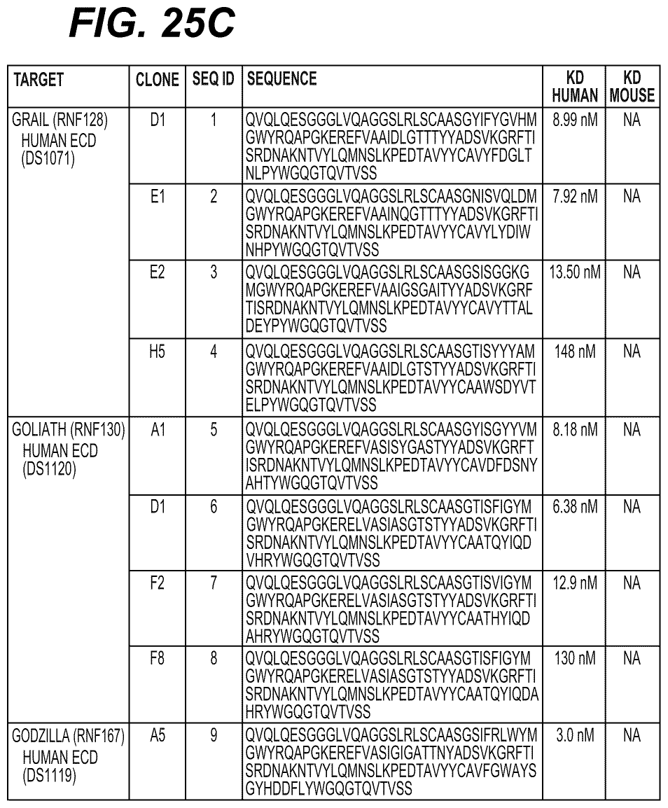

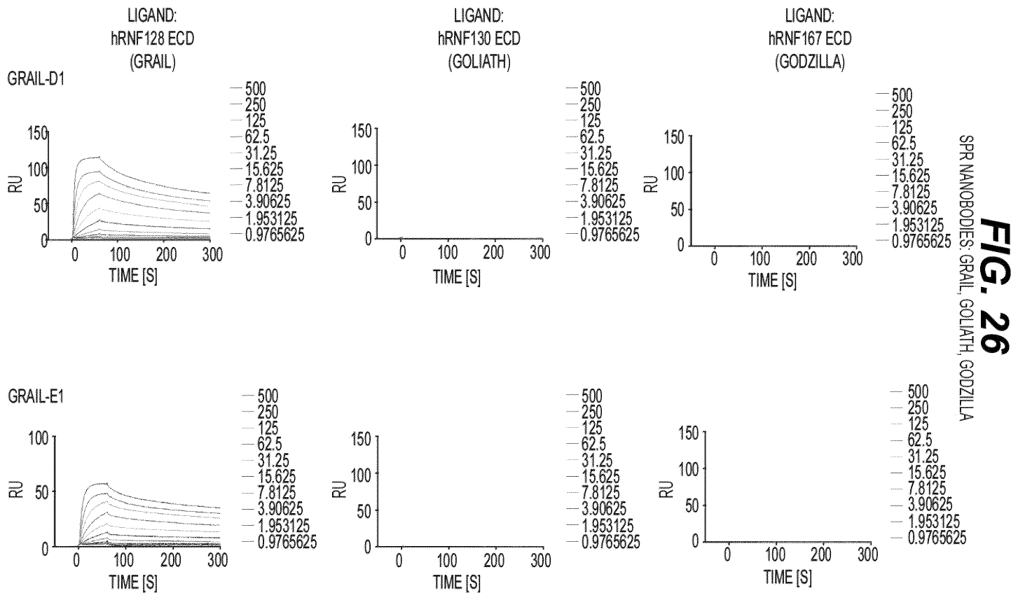

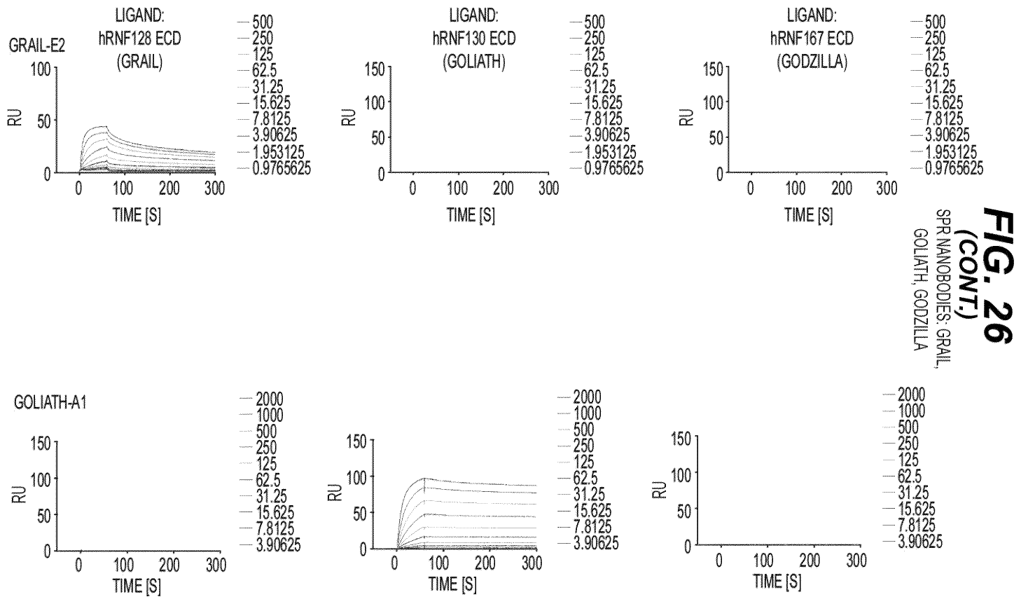

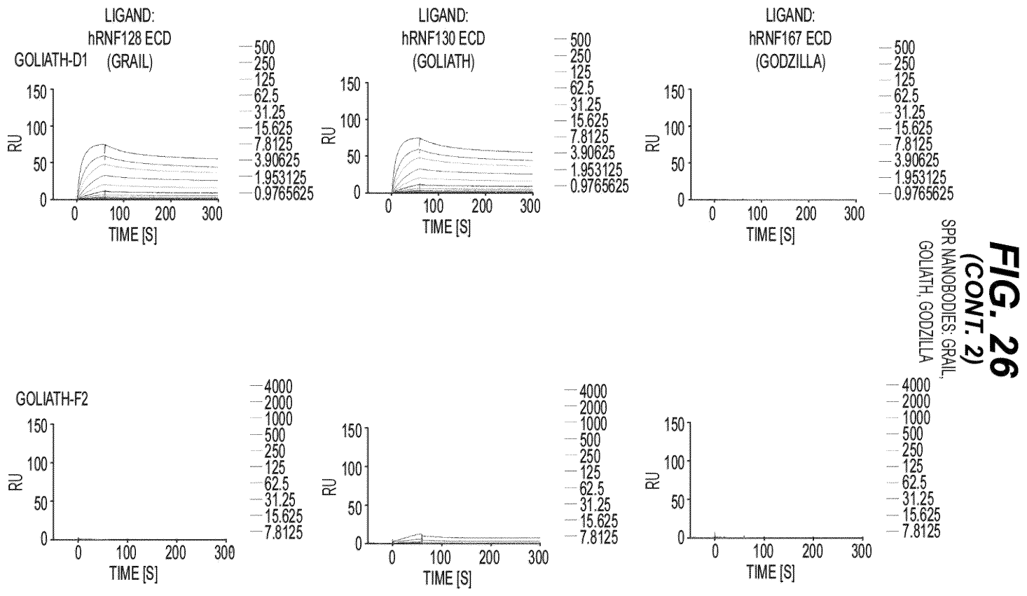

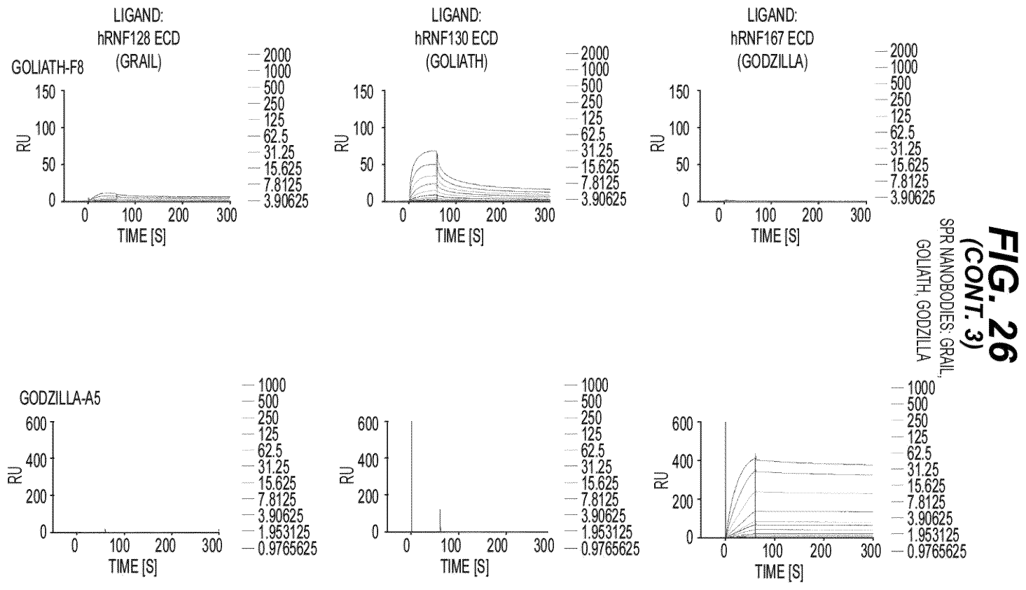

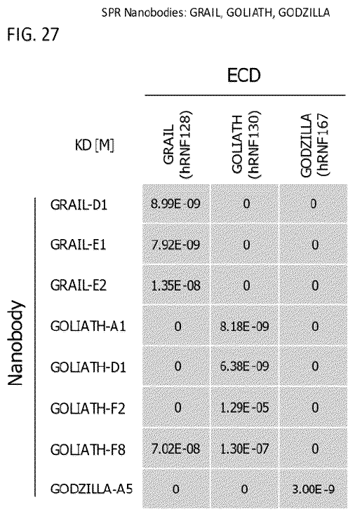

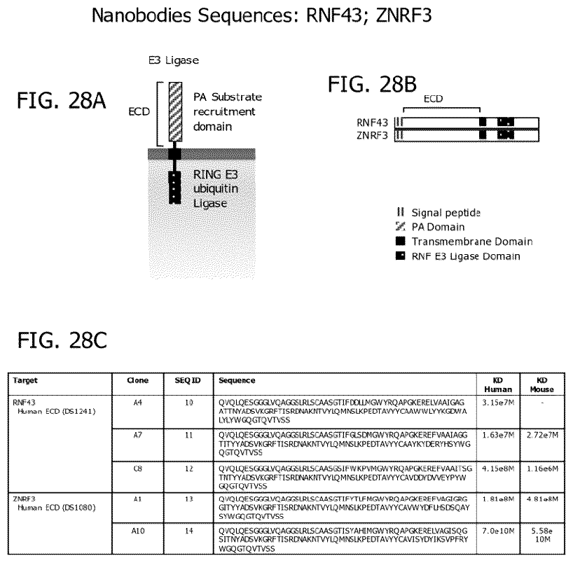

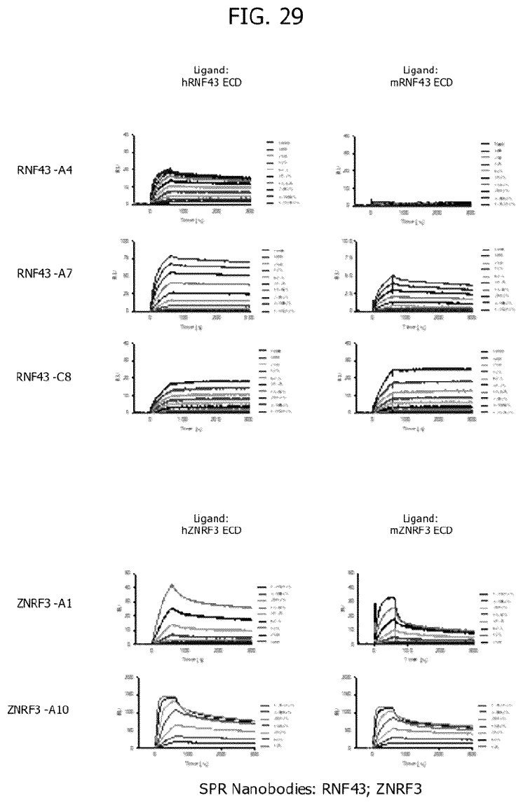

FIG. 24 depicts schematical representations of exemplary REULR constructs of the present disclosure. As shown in FIG. 24A, a REULR construct comprised of a VHH specific for a target is linked to a VHH specific for a E3 Ub ligase. As shown in FIG. 24B, a REULR construct comprised of two VHHs, each being specific for a target, linked to a VHH specific for a E3 Ub ligase. Each of the two VHHs specific for a target may be specific for the same or different targets. Thus, exemplary REULR constructs of the present disclosure may target one or more different target proteins, or two or more different target proteins. As shown in FIG. 24C, a REULR construct comprised of a VHH specific for a target is linked to two VHHs, each comprised of a VHH specific for a E3 Ub ligase. Thus, exemplary REULR constructs of the present disclosure may target two or more E3 Ub ligase proteins.

As shown in FIG. 24D, a REULR construct comprised of three VHHs, each being specific for a target, linked to three VHHs, each specific for a E3 Ub ligase. Thus, an exemplary REULR construct of the present disclosure may target 1-3 or more E3 Ub ligase proteins and/or 1-3 or more target proteins. The dotted lines represent optional additional VHHs and while three VHHs on each side are depicted, there may be more or fewer than three VHHs and the resulting REULR construct is not necessarily symmetrical such that there may be more or fewer VHHs specific for one or more target proteins than the number of VHHs specific for E3 Ub ligase proteins. While the linker in this example is depicted as a hub and spoke patten, this is for ease of representation only and is not at all required. Amino acid based or chemical based linkers are contemplated within the scope of the presently described linkers. Such linkers may be provided such that they ligate three or more VHHs together in the functional manner contemplated herein. As used herein, “linker” and “linker molecule” are used interchangeably and refer to any of the contemplated linker moieties contemplated herein.

As described herein, any of a variety of linker moieties may be included in the presently described REULR constructs. A linker may be a molecule or group of molecules. The linker may be comprised of a single linking molecule or may comprise a linking molecule and a spacer molecule, intended to separate the linking molecule and a compound by a specific distance. For example, linkers may comprise an amino acid sequence, which amino acid sequence does not interfere with the activity or binding of the ligated VHHs. While a length for a linker is necessarily included with the specific examples provided herein, this length and amino acid composition is exemplary only. Such linkers may be derived from natural human sequences, antibody linker sequences, or comprise synthetic sequences. These linkers may be long or short and adapted to the application based, for example, on the E3 Ub ligase or ligases on the REULR construct.

For example, while not intending to be bound by a theory of operation, it is submitted based on the data herein that varying the linker length may be provided to modulate the outcome of the REULR construct. In examples where the linker is shorter, or otherwise in closer proximity to the target protein (e.g., receptor), this translate into higher turnover of the target protein (e.g., receptor). Also, in examples where the linker is longer, or otherwise in relative to the prior sentence, further in proximity from the target protein (e.g., receptor), this translate into lower turnover of the target protein (e.g., receptor).

As described herein, the described REULR constructs may be used in a variety of therapeutic contexts. For example, certain target proteins include proto-oncogenic and oncogenic receptors, which are signaling receptors that arise as a result of mutations that increase the expression level or activity of a proto-oncogene. Underlying genetic mechanisms associated with oncogene activation can include point mutations, deletions, or insertions among others, that lead to a hyperactive gene product with detrimental outcome leading to cancer. Modulating these receptors is contemplated herein, for example, to reduce the deleterious effect of these receptors, prevent disease, or therapeutically intervene with positive health effects. Prominent oncogenic receptor families include but are not limited to: The ErbB family of receptor tyrosine kinases (RTKs) ERBB1 (EGFR), ERBB2, ERBB3, and ERBB4 (also known as HER1, HER2, HER3, and HER4). The neu oncogenic receptor, insulin receptor substrate 4 IRS4/insulin receptor/c-ros proto-oncogenic receptor, insulin and IGF-1 and -II/oncogenic IGF-1R, oncogenic PDGFAR, oncogenic TEL/PDGFRB, HGF/HGFR(MET oncogenic receptor). The oncogenic TAM family of kinases including AXL (UFO), TYRO3 and MERTK. The cytokine receptor rc family members (IL-2-BCM fusion,IL-3/oncogenic IL-3Ra,IL-7/oncogenic IL-7R and IL-21R-BCL6 fusion). And cytokine receptors including the βc family (G-CSF/oncogenic CSF3R, oncogenic EPOR), and oncogenic autocrine growth hormone/nuclear GHR and other VEGFR2. Among others, including others contemplated herein.

Tumor associated antigens are antigenic substance produced in tumor cells triggering an immune response in the host. Tumor antigens are potential candidates for use in cancer therapy and can be broadly classified into: Products of Mutated Oncogenes and Tumor Suppressor Genes, products of other mutated genes, overexpressed or aberrantly expressed cellular proteins, tumor antigens produced by oncogenic viruses, oncofetal antigens, altered cell Surface glycolipids and glycoproteins cell type-specific differentiation Antigens. These can include members of the Immune checkpoint B7 receptor family: B7-1 (CD80), B7-2 (CD86), B7-DC (PD-L2), B7-H1 (PD-1), B7-H2 (ICOSLG), B7-H3 (CD276), B7-H4 (VTCN1), B7-H5 (VISTA), B7-H6 (NCR3LG1), B7-H7 (HHLA2).

Table 1 depicts the amino acid sequences of certain exemplary single variable domain heavy chain antibodies (VHH/nanobody) that specifically bind the ECDs of the human and mouse E3 Ub ligases GRAIL (RNF128), GOLIATH (RNF130), GODZILLA (RNF167), RNF43 and ZNRF3. Table 1 also includes measured equilibrium dissociation constants for specified VHHs. Non-recited equilibrium dissociation constants can be readily determined by methods available in the art based on the structural information provided herein. The exemplified polypeptide sequence embodying the VHH to the target protein may have alterations in sequence with the proviso that such alterations do not remove its specificity for the target protein, while variability in terms of increased binding affinity, and reduced binding affinity while maintaining specificity, are contemplated. Other and different linkers/linker molecules are contemplated and, in that regard, the specific examples are not intended to be limiting.

TABLE 1

| | | SEQ | |
|---|---|---|---|
| | | ID | |
| Target | Sequence | NO | K_{D} |
| GRAIL | QVQLQESGGGLVQAGGSLRL | 1 | 8.99 nM |
| (RNF128) | SCAASGYIFYGVHMGWYRQA | | |
| | PGKEREFVAAIDLGTTTYYA | | |
| | DSVKGRFTISRDNAKNTVYL | | |
| | QMNSLKPEDTAVYYCAVYFD | | |
| | GLTNLPYWGQGTQVTVSS | | |
| | QVQLQESGGGLVQAGGSLRL | 2 | 7.92 nM |
| | SCAASGNISVQLDMGWYRQA | | |
| | PGKEREFVAAINQGTTTYYA | | |
| | DSVKGRFTISRDNAKNTVYL | | |
| | QMNSLKPEDTAVYYCAVYLY | | |
| | DIWNHPYWGQGTQVTVSS | | |
| | QVQLQESGGGLVQAGGSLRL | 3 | 13.50 nM |
| | SCAASGSISGGKGMGWYRQA | | |
| | PGKEREFVAAIGSGAITYYA | | |
| | DSVKGRFTISRDNAKNTVYL | | |
| | QMNSLKPEDTAVYYCAVYTT | | |
| | ALDEYPYWGQGTQVTVSS | | |
| | QVQLQESGGGLVQAGGSLRL | 4 | 148 nM |
| | SCAASGTISYYYAMGWYRQA | | |
| | PGKEREFVAAIDLGTSTYYA | | |
| | DSVKGRFTISRDNAKNTVYL | | |
| | QMNSLKPEDTAVYYCAAWSD | | |
| | YVTELPYWGQGTQVTVSS | | |
| GOLIATH | QVQLQESGGGLVQAGGSLRL | 5 | 8.18 nM |
| (RNF130) | SCAASGYISGYYVMGWYRQA | | |
| | PGKEREFVASISYGASTYYA | | |
| | DSVKGRFTISRDNAKNTVYL | | |
| | QMNSLKPEDTAVYYCAVDFD | | |
| | SNYAHTYWGQGTQVTVSS | | |
| | QVQLQESGGGLVQAGGSLRL | 6 | 6.38 nM |
| | SCAASGTISFIGYMGWYRQA | | |
| | PGKERELVASIASGTSTYYA | | |
| | DSVKGRFTISRDNAKNTVYL | | |
| | QMNSLKPEDTAVYYCAATQY | | |
| | IQDVHRYWGQGTQVTVSS | | |
| | QVQLQESGGGLVQAGGSLRL | 7 | 12.9 uM |
| | SCAASGTISVIGYMGWYRQA | | |
| | PGKERELVASIASGTSTYYA | | |
| | DSVKGRFTISRDNAKNTVYL | | |
| | QMNSLKPEDTAVYYCAATHY | | |
| | IQDAHRYWGQGTQVTVSS | | |
| | QVQLQESGGGLVQAGGSLRL | 8 | 130 nM |
| | SCAASGTISFIGYMGWYRQA | | |
| | PGKERELVASIASGTSTYYA | | |
| | DSVKGRFTISRDNAKNTVYL | | |
| | QMNSLKPEDTAVYYCAATQY | | |
| | IQDAHRYWGQGTQVTVSS | | |
| GODZILLA | QVQLQESGGGLVQAGGSLRL | 9 | 3.0 nM |
| (RNF167) | SCAASGSIFRLWYMGWYRQA | | |
| | PGKEREFVASIGIGATTNYA | | |
| | DSVKGRFTISRDNAKNTVYL | | |
| | QMNSLKPEDTAVYYCAVFGW | | |
| | AYSGYHDDFLYWGQGTQVTV | | |
| | SS | | |
| RNF43 | QVQLQESGGGLVQAGGSLRL | 10 | 315 nM |
| | SCAASGTIFDDLLMGWYRQA | | |
| | PGKERELVAAIGAGATTNYA | | |
| | DSVKGRFTISRDNAKNTVYL | | |
| | QMNSLKPEDTAVYYCAAWWL | | |
| | YYKGDWALYLYWGQGTQVTV | | |
| | SS | | |
| | QVQLQESGGGLVQAGGSLRL | 11 | 163 nM |
| | SCAASGTIFGLSDMGWYRQA | | |
| | PGKEREFVAAIAGGTITYYA | | |
| | DSVKGRFTISRDNAKNTVYL | | |
| | QMNSLKPEDTAVYYCAAYKY | | |
| | DERYHSYWGQGTQVTVSS | | |
| | QVQLQESGGGLVQAGGSLRL | 12 | 41.5 nM |
| | SCAASGSIFWKPVMGWYRQA | | |
| | PGKEREFVAAITSGTNTYYA | | |
| | DSVKGRFTISRDNAKNTVYL | | |
| | QMNSLKPEDTAVYYCAVDDY | | |
| | DVVEYPYWGQGTQVTVSS | | |
| ZNRF3 | QVQLQESGGGLVQAGGSLRL | 13 | 18.1 nM |
| | SCAASGTIFYTLFMGWYRQA | | |
| | PGKEREFVAGIGRGGITYYA | | |
| | DSVKGRFTISRDNAKNTVYL | | |
| | QMNSLKPEDTAVYYCAVWYD | | |
| | FLHSDSQAYSYWGQGTQVTV | | |
| | SS | | |
| | QVQLQESGGGLVQAGGSLRL | 14 | 0.70 nM |
| | SCAASGTISYAHIMGWYRQA | | |
| | PGKERELVAGISQGSITNYA | | |
| | DSVKGRFTISRDNAKNTVYL | | |
| | QMNSLKPEDTAVYYCAVISY | | |
| | DYIKSVPFRYWGQGTQVTVS | | |
| | S | | |
| PD-1 VHH | DVQLVESGGGVVQPGGSLRL | 15 | |
| | SCAASGSIASIHAMGWFRQA | | |
| | PGKEREFVAVITWSGGITYY | | |
| | ADSVKGRFTISRDNSKNTVY | | |
| | LQMNSLRPEDTALYYCAGDK | | |
| | HQSSWYDYWGQGTLVTVSS | | |
| EGFR- | QVKLEESGGGSVQTGGSLRL | 16 | |
| 7D12 | TCAASGRTSRSYGMGWFRQA | | |
| | PGKEREFVSGISWRGDSTGY | | |
| | ADSVKGRFTISRDNAKNTVD | | |
| | LQMNSLKPEDTAIYYCAAAA | | |
| | GSAWYGTLYEYDYWGQGTQV | | |
| | TVSS | | |
| EGFR-9G8 | VQLVESGGGLVQAGGSLRLS | 17 | |
| | CAASGRTFSSYAMGWFRQAP | | |
| | GKEREFVVAINWSSGSTYYA | | |
| | DSVKGRFTISRDNAKNTMYL | | |
| | QMNSLKPEDTAVYYCAAGYQ | | |
| | INSGNYNFKDYEYDYWGQGT | | |
| | QVTVSS | | |
| vMIP-II | GDTLGASWHRPDKCCLGYQK | 18 | |
| | RPLPQVLLSSWYPTSQLCSK | | |
| | PGVIFLTKRGRQVCADKSKD | | |
| | WVKKLMQQLPVTAR | | |
| MT7 | LTCVKSNSIWFPTSEDCPDG | 19 | |
| | QNLCFKRWQYISPRMYDFTR | | |
| | GCAATCPKAEYRDVINCCGT | | |
| | DKCNK | | |
| TX24 | LTCVKSNSIRFPTSGDCPDG | 20 | |
| | QNLCFKRWQSPGMPRPMWAL | | |
| | VCAATCPKAPPNEDINCCGT | | |
| | DKCNK | | |
| mPD-1- | DIVLTQSTLPLPVSPGESVS | 21 | |
| RMP | ITCRSSKSLLYSDGKTYLNW | | |
| | YLQRPGQSPQLLIYWMSTRA | | |
| | SGVSDRFSGSGSGTDFTLKI | | |
| | SGVEAEDVGIYYCQQGLEFP | | |
| | TFGGGTKLELKGGSTRSSSS | | |
| | GGGGSGGGGEVQLQESGPGL | | |
| | VKPSQSLSLTCSVTGYSITS | | |
| | SYRWNWIRQFPGNRLEWMGY | | |
| | INSAGISNYNPSLKRRISIT | | |
| | RDTSKNQFFLQVNSVTTEDT | | |
| | ATYYCARSDNMGTTPFTYWG | | |
| | QGTLVTVSS | | |
| mEGFR- | EVMLVESGGVLVKPGGSLKL | 22 | |
| 7A7 | SCAASGFTFSRYAMSWVRQT | | |
| | PEKRLEWVATISSGGSYSYY | | |
| | PDSVKGRFTISRDNVKNTLY | | |
| | LQMSSLRSEDTAMYYCARDS | | |
| | GGFAYWGQWGQGTLVTVSAG | | |
| | GGGSGGGGSGGGGSDIQMTQ | | |
| | TTSSLSASLGDRVTISCRAS | | |
| | QDISNYLNWYQQKPDGTVKL | | |
| | LIYYTSRLHSGVTSRFSGSG | | |
| | SGTDYSLTISNLEQEDIATY | | |
| | FCQQGNTLPWTFGGGTKKVE | | |
| | IKRAD | | |

Table 2 depicts the amino acid sequences of exemplary Fratricide REULR constructs specific for the ECD of the E3 Ub ligase GRAIL (RNF128), RNF43 and or ZNRF3. Two single variable domain heavy chain antibodies are connected with a 3C linker LEVLFQGP (SEQ ID NO: 73) or a GS flanked 3C linker GSLEVLFQGPGS (SEQ ID NO: 106) that is designated by the underlined portion. In the example represented by SEQ ID NO: 23, the amino acid sequence of SEQ ID NO: 2 is present on both the N-terminal side and the C-terminal side of the 3C linker LEVLFQGP (SEQ ID NO: 73). In the example represented by SEQ ID NO: 24, the amino acid sequence of SEQ ID NO: 3 is present on the N-terminal side and the C-terminal side of the 3C linker LEVLFQGP (SEQ ID NO: 73). In the example represented by SEQ ID NO: 97, the amino acid sequence of SEQ ID NO: 10 is present on the N-terminal side and the C-terminal side of the 3C linker GSLEVLFQGPGS (SEQ ID NO: 106). In the example represented by SEQ ID NO: 98, the amino acid sequence of SEQ ID NO: 11 is present on the N-terminal side and the C-terminal side of the 3C linker GSLEVLFQGPGS (SEQ ID NO: 106). In the example represented by SEQ ID NO: 99, the amino acid sequence of SEQ ID NO: 12 is present on the N-terminal side and the C-terminal side of the 3C linker GSLEVLFQGPGS (SEQ ID NO: 106). In the example represented by SEQ ID NO: 100, the amino acid sequence of SEQ ID NO: 13 is present on the N-terminal side and the C-terminal side of the 3C linker GSLEVLFQGPGS (SEQ ID NO: 106). In the example represented by SEQ ID NO: 101, the amino acid sequence of SEQ ID NO: 14 is present on the N-terminal side and the C-terminal side of the 3C linker GSLEVLFQGPGS (SEQ ID NO: 106). In the example represented by SEQ ID NO: 102, the amino acid sequence of SEQ ID NO: 11 is present on the N-terminal side and the amino acid sequence of SEQ ID NO: 14 is present on the C-terminal side of the 3C linker GSLEVLFQGPGS (SEQ ID NO: 106). In the example represented by SEQ ID NO: 103, the amino acid sequence of SEQ ID NO: 11 is present on the N-terminal side and the amino acid sequence of SEQ ID NO: 13 is present on the C-terminal side of the 3C linker GSLEVLFQGPGS (SEQ ID NO: 106). In the example represented by SEQ ID NO: 104, the amino acid sequence of SEQ ID NO: 12 is present on the N-terminal side and the amino acid sequence of SEQ ID NO: 14 is present on the C-terminal side of the 3C linker GSLEVLFQGPGS (SEQ ID NO: 106). In the example represented by SEQ ID NO: 105, the amino acid sequence of SEQ ID NO: 12 is present on the N-terminal side and the amino acid sequence of SEQ ID NO: 13 is present on the C-terminal side of the 3C linker GSLEVLFQGPGS (SEQ ID NO: 106).

The exemplified polypeptide sequence embodying the VHH to the target protein may have alterations in sequence with the proviso that such alterations do not remove its specificity for the target protein, while variability in terms of increased binding affinity, and reduced binding affinity while maintaining specificity, are contemplated. Other and different linkers/linker molecules are contemplated and, in that regard, the specific examples are not intended to be limiting.

TABLE 2

| | | SEQ |
|---|---|---|
| | | ID |
| Target | Sequence | NO |
| GRAIL- | QVQLQESGGGLVQAGGSLRLSCAASGNISVQLDMGWYRQAPGK | 23 |
| GRAIL | EREFVAAINQGTTTYYADSVKGRFTISRDNAKNTVYLQMNSLKP | |
| | EDTAVYYCAVYLYDIWNHPYWGQGTQVTVSSLEVLFQGPQVQL | |
| | QESGGGLVQAGGSLRLSCAASGNISVQLDMGWYRQAPGKEREF | |
| | VAAINQGTTTYYADSVKGRFTISRDNAKNTVYLQMNSLKPEDTA | |
| | VYYCAVYLYDIWNHPYWGQGTQVTVSS | |
| | QVQLQESGGGLVQAGGSLRLSCAASGSISGGKGMGWYRQAPGK | 24 |
| | EREFVAAIGSGAITYYADSVKGRFTISRDNAKNTVYLQMNSLKPE | |
| | DTAVYYCAVYTTALDEYPYWGQGTQVTVSSLEVLFQGPQVQLQ | |
| | ESGGGLVQAGGSLRLSCAASGSISGGKGMGWYRQAPGKEREFV | |
| | AAIGSGAITYYADSVKGRFTISRDNAKNTVYLQMNSLKPEDTAV | |
| | YYCAVYTTALDEYPYWGQGTQVTVSS | |
| RNF43- | QVQLQESGGGLVQAGGSLRLSCAASGTIFDDLLMGWYRQAPGK | 97 |
| RNF43 | ERELVAAIGAGATTNYADSVKGRFTISRDNAKNTVYLQMNSLKP | |
| | EDTAVYYCAAWWLYYKGDWALYLYWGQGTQVTVSSGSLEVLF | |
| | QGPGSQVQLQESGGGLVQAGGSLRLSCAASGTIFDDLLMGWYR | |
| | QAPGKERELVAAIGAGATTNYADSVKGRFTISRDNAKNTVYLQM | |
| | NSLKPEDTAVYYCAAWWLYYKGDWALYLYWGQGTQVTVSS | |
| RNF43- | QVQLQESGGGLVQAGGSLRLSCAASGTIFGLSDMGWYRQAPGK | 98 |
| RNF43 | EREFVAAIAGGTITYYADSVKGRFTISRDNAKNTVYLQMNSLKPE | |
| | DTAVYYCAAYKYDERYHSYWGQGTQVTVSSGSLEVLFQGPGSQ | |
| | VQLQESGGGLVQAGGSLRLSCAASGTIFGLSDMGWYRQAPGKER | |
| | EFVAAIAGGTITYYADSVKGRFTISRDNAKNTVYLQMNSLKPEDT | |
| | AVYYCAAYKYDERYHSYWGQGTQVTVSS | |
| | QVQLQESGGGLVQAGGSLRLSCAASGSIFWKPVMGWYRQAPGK | 99 |
| | EREFVAAITSGTNTYYADSVKGRFTISRDNAKNTVYLQMNSLKPE | |
| | DTAVYYCAVDDYDVVEYPYWGQGTQVTVSSGSLEVLFQGPGSQ | |
| | VQLQESGGGLVQAGGSLRLSCAASGSIFWKPVMGWYRQAPGKE | |
| | REFVAAITSGTNTYYADSVKGRFTISRDNAKNTVYLQMNSLKPE | |
| | DTAVYYCAVDDYDVVEYPYWGQGTQVTVSS | |
| ZNRF3- | QVQLQESGGGLVQAGGSLRLSCAASGTIFYTLFMGWYRQAPGKE | 100 |
| ZNRF3 | REFVAGIGRGGITYYADSVKGRFTISRDNAKNTVYLQMNSLKPED | |
| | TAVYYCAVWYDFLHSDSQAYSYWGQGTQVTVSSGSLEVLFQGPG | |
| | SQVQLQESGGGLVQAGGSLRLSCAASGTIFYTLFMGWYRQAPGK | |
| | EREFVAGIGRGGITYYADSVKGRFTISRDNAKNTVYLQMNSLKP | |
| | EDTAVYYCAVWYDFLHSDSQAYSYWGQGTQVTVSS | |
| | QVQLQESGGGLVQAGGSLRLSCAASGTISYAHIMGWYRQAPGKE | 101 |
| | RELVAGISQGSITNYADSVKGRFTISRDNAKNTVYLQMNSLKPED | |
| | TAVYYCAVISYDYIKSVPFRYWGQGTQVTVSSGSLEVLFQGPGSQ | |
| | VQLQESGGGLVQAGGSLRLSCAASGTISYAHIMGWYRQAPGKER | |
| | ELVAGISQGSITNYADSVKGRFTISRDNAKNTVYLQMNSLKPEDT | |
| | AVYYCAVISYDYIKSVPFRYWGQGTQVTVSS | |
| RNF43- | QVQLQESGGGLVQAGGSLRLSCAASGTIFGLSDMGWYRQAPGK | 102 |
| ZNRF3 | EREFVAAIAGGTITYYADSVKGRFTISRDNAKNTVYLQMNSLKPE | |
| | DTAVYYCAAYKYDERYHSYWGQGTQVTVSSGSLEVLFQGPGSQ | |
| | VQLQESGGGLVQAGGSLRLSCAASGTISYAHIMGWYRQAPGKER | |
| | ELVAGISQGSITNYADSVKGRFTISRDNAKNTVYLQMNSLKPEDT | |
| | AVYYCAVISYDYIKSVPFRYWGQGTQVTVSS | |
| | QVQLQESGGGLVQAGGSLRLSCAASGTIFGLSDMGWYRQAPGK | 103 |
| | EREFVAAIAGGTITYYADSVKGRFTISRDNAKNTVYLQMNSLKPE | |
| | DTAVYYCAAYKYDERYHSYWGQGTQVTVSSGSLEVLFQGPGSQ | |
| | VQLQESGGGLVQAGGSLRLSCAASGTIFYTLFMGWYRQAPGKER | |
| | EFVAGIGRGGITYYADSVKGRFTISRDNAKNTVYLQMNSLKPEDT | |
| | AVYYCAVWYDFLHSDSQAYSYWGQGTQVTVSS | |
| | QVQLQESGGGLVQAGGSLRLSCAASGSIFWKPVMGWYRQAPGK | 104 |
| | EREFVAAITSGTNTYYADSVKGRFTISRDNAKNTVYLQMNSLKPE | |
| | DTAVYYCAVDDYDVVEYPYWGQGTQVTVSSGSLEVLFQGPGSQ | |
| | VQLQESGGGLVQAGGSLRLSCAASGTISYAHIMGWYRQAPGKER | |
| | ELVAGISQGSITNYADSVKGRFTISRDNAKNTVYLQMNSLKPEDT | |
| | AVYYCAVISYDYIKSVPFRYWGQGTQVTVSS | |
| | QVQLQESGGGLVQAGGSLRLSCAASGSIFWKPVMGWYRQAPGK | 105 |
| | EREFVAAITSGTNTYYADSVKGRFTISRDNAKNTVYLQMNSLKPE | |
| | DTAVYYCAVDDYDVVEYPYWGQGTQVTVSSGSLEVLFQGPGSQ | |
| | VQLQESGGGLVQAGGSLRLSCAASGTIFYTLFMGWYRQAPGKER | |
| | EFVAGIGRGGITYYADSVKGRFTISRDNAKNTVYLQMNSLKPEDT | |
| | AVYYCAVWYDFLHSDSQAYSYWGQGTQVTVSS | |

For clarity and not by means of limitation, the present disclosure contemplates any combination of single variable domain heavy chain antibody amino acid sequences on the N-terminal and the C-terminal portions of contemplated linkers such as the 3C linker of SEQ ID NO: 73 or the 3C linker of SEQ ID NO: 106. For example, a REULR construct of the present disclosure may be represented by the following formula: X-L-Wherein X is selected from any one of SEQ ID NO: 1-22, wherein X is selected from any one of SEQ ID NO: 1-22, and wherein L is selected from SEQ ID NO: 73 or another linker such as a 3C linker. In certain embodiments an additional 1-5 amino acids is present between X and L and/or between L and Y, for example as shown in Tables 10-11.

Further, the 3C linkers are used in the presently provided examples and embodiments for ease of demonstrating functionality of the exemplified REULR constructs. As a person of ordinary skill in the art would appreciate, the use of 3C protease will cleave the 3C linker in a predictable manner thereby providing evidence of the effectiveness of intact REULR constructs compared with REULR constructs with a cleaved linker. In this regard, it is contemplated and described herein that REULR constructs of the present disclosure incorporate a linker that is a non-3C linker. Such linker permits simultaneous binding of the VHHs of intact REULR constructs with the E3 Ubiquitin Ligase as well as the target protein.

Table 3 depicts the amino acid sequences of exemplary REULR constructs specific for the ECD of the E3 Ub ligase GRAIL (RNF128) and the ECD of the PD-1 receptor. Two single variable domain heavy chain antibodies are connected with the 3C linker LEVLFQGP (SEQ ID NO: 73) that is designated by the underlined portion. In the example represented by SEQ ID NO: 25, the amino acid sequence of SEQ ID NO: 15 is present on the N-terminal side of the 3C linker and the amino acid sequence of SEQ ID NO: 2 is present on the C-terminal side of the 3C linker. In the example represented by SEQ ID NO: 26, the amino acid sequence of SEQ ID NO: 15 is present on the N-terminal side of the 3C linker and the amino acid sequence of SEQ ID NO: 3 is present on the C-terminal side of the 3C linker. In the example represented by SEQ ID NO: 27, the amino acid sequence of SEQ ID NO: 2 is present on the N-terminal side of the 3C linker and the amino acid sequence of SEQ ID NO: 15 is present on the C-terminal side of the 3C linker. In the example represented by SEQ ID NO: 28, the amino acid sequence of SEQ ID NO: 3 is present on the N-terminal side of the 3C linker and the amino acid sequence of SEQ ID NO: 15 is present on the C-terminal side of the 3C linker. While Table 3 depicts the presence of the 3C linker of SEQ ID NO: 73, the 3C linker of SEQ ID NO: 106 may be provided in replacement of the 3C linker of SEQ ID NO: 73.

It is specially contemplated that the specifically exemplified REULR constructs are exemplary only. The exemplified polypeptide sequence embodying the VHH to the target protein may have alterations in sequence with the proviso that such alterations do not remove its specificity for the target protein, while variability in terms of increased binding affinity, and reduced binding affinity while maintaining specificity, are contemplated. Other and different linkers/linker molecules are contemplated and, in that regard, the specific examples are not intended to be limiting. Further, other VHHs specific for other E3 Ubiquitin Ligases may be substituted, for example, VHHs specific for a RING type E3 Ubiquitin Ligase, including GRAIL, GOLIATH and/or GODZILLA, and/or VHHs specific for a E3 Ubiquitin Ligase such as RNF133, RNF148, RNF128, RNF149, RNF130, RNF150, RNF122, RNF43, ZNRF3, ZNRF4, RNF13, RNF167, AMFR, STVN1, RNF170, RNF121, RNF175, RNF139, RNF145, MARCHF5, VFPL1, RNFT1, RNF180, RNF103, RNF182, RNF5, RNF185, RNF19A, RNF19B, RNF144A, RNF144B, RNF217, MARCHF1, MARCHF8, MARCHF2, MARCHF3, MARCHF11, MARCHF4, MARCHF9, MARCHF6, NR1H4, RNF126, DCST1, RNF152, RNF186, CGRRF1, MUL1, TRIM13, TRIM59, and/or RNF112.

TABLE 3

| | | SEQ |
|---|---|---|
| | | ID |
| Target | Sequence | NO |
| PD-1- | DVQLVESGGGVVQPGGSLRLSCAASGSIASIHAMGWFRQAPGKE | 25 |
| GRAIL | REFVAVITWSGGITYYADSVKGRFTISRDNSKNTVYLQMNSLRPE | |
| | DTALYYCAGDKHQSSWYDYWGQGTLVTVSSLEVLFQGPQVQLQ | |
| | ESGGGLVQAGGSLRLSCAASGNISVQLDMGWYRQAPGKEREFV | |
| | AAINQGTTTYYADSVKGRFTISRDNAKNTVYLQMNSLKPEDTAV | |
| | YYCAVYLYDIWNHPYWGQGTQVTVSS | |
| | DVQLVESGGGVVQPGGSLRLSCAASGSIASIHAMGWFRQAPGKE | 26 |
| | REFVAVITWSGGITYYADSVKGRFTISRDNSKNTVYLQMNSLRPE | |
| | DTALYYCAGDKHQSSWYDYWGQGTLVTVSSLEVLFQGPQVQLQ | |
| | ESGGGLVQAGGSLRLSCAASGSISGGKGMGWYRQAPGKEREFV | |
| | AAIGSGAITYYADSVKGRFTISRDNAKNTVYLQMNSLKPEDTAV | |
| | YYCAVYTTALDEYPYWGQGTQVTVSS | |
| GRAIL- | QVQLQESGGGLVQAGGSLRLSCAASGNISVQLDMGWYRQAPGK | 27 |
| PD-1 | EREFVAAINQGTTTYYADSVKGRFTISRDNAKNTVYLQMNSLKP | |
| | EDTAVYYCAVYLYDIWNHPYWGQGTQVTVSSLEVLFQGPDVQL | |
| | VESGGGVVQPGGSLRLSCAASGSIASIHAMGWFRQAPGKEREFV | |
| | AVITWSGGITYYADSVKGRFTISRDNSKNTVYLQMNSLRPEDTAL | |
| | YYCAGDKHQSSWYDYWGQGTLVTVSS | |
| | QVQLQESGGGLVQAGGSLRLSCAASGSISGGKGMGWYRQAPGK | 28 |
| | EREFVAAIGSGAITYYADSVKGRFTISRDNAKNTVYLQMNSLKPE | |
| | DTAVYYCAVYTTALDEYPYWGQGTQVTVSSLEVLFQGPDVQLV | |
| | ESGGGVVQPGGSLRLSCAASGSIASIHAMGWFRQAPGKEREFVA | |
| | VITWSGGITYYADSVKGRFTISRDNSKNTVYLQMNSLRPEDTALY | |
| | YCAGDKHQSSWYDYWGQGTLVTVSS | |

Table 4 depicts the amino acid sequences of exemplary REULR constructs specific for the ECD of the E3 Ub ligase GRAIL (RNF128) and the ECD of the EGFR receptor. Two single variable domain heavy chain antibodies are connected with the 3C linker LEVLFQGP (SEQ ID NO: 73) that is designated by the underlined portion. In the example represented by SEQ ID NO: 29, the amino acid sequence of SEQ ID NO: 16 is present on the N-terminal side of the 3C linker and the amino acid sequence of SEQ ID NO: 2 is present on the C-terminal side of the 3C linker. In the example represented by SEQ ID NO: 30, the amino acid sequence of SEQ ID NO: 16 is present on the N-terminal side of the 3C linker and the amino acid sequence of SEQ ID NO: 3 is present on the C-terminal side of the 3C linker. In the example represented by SEQ ID NO: 31, the amino acid sequence of SEQ ID NO: 17 is present on the N-terminal side of the 3C linker and the amino acid sequence of SEQ ID NO: 2 is present on the C-terminal side of the 3C linker. In the example represented by SEQ ID NO: 32, the amino acid sequence of SEQ ID NO: 17 is present on the N-terminal side of the 3C linker and the amino acid sequence of SEQ ID NO: 3 is present on the C-terminal side of the 3C linker. In the example represented by SEQ ID NO: 33, the amino acid sequence of SEQ ID NO: 2 is present on the N-terminal side of the 3C linker and the amino acid sequence of SEQ ID NO: 16 is present on the C-terminal side of the 3C linker. In the example represented by SEQ ID NO: 34, the amino acid sequence of SEQ ID NO: 3 is present on the N-terminal side of the 3C linker and the amino acid sequence of SEQ ID NO: 16 is present on the C-terminal side of the 3C linker. In the example represented by SEQ ID NO: 35, the amino acid sequence of SEQ ID NO: 2 is present on the N-terminal side of the 3C linker and the amino acid sequence of SEQ ID NO: 17 is present on the C-terminal side of the 3C linker. In the example represented by SEQ ID NO: 36, the amino acid sequence of SEQ ID NO: 3 is present on the N-terminal side of the 3C linker and the amino acid sequence of SEQ ID NO: 17 is present on the C-terminal side of the 3C linker. While Table 4 depicts the presence of the 3C linker of SEQ ID NO: 73, the 3C linker of SEQ ID NO: 106 may be provided in replacement of the 3C linker of SEQ ID NO: 73.

It is specially contemplated that the specifically exemplified REULR constructs are exemplary only. The exemplified polypeptide sequence embodying the VHH to the target protein may have alterations in sequence with the proviso that such alterations do not remove its specificity for the target protein, while variability in terms of increased binding affinity, and reduced binding affinity while maintaining specificity, are contemplated. Other and different linkers/linker molecules are contemplated and, in that regard, the specific examples are not intended to be limiting. Further, other VHHs specific for other E3 Ubiquitin Ligases may be substituted, for example, VHHs specific for a RING type E3 Ubiquitin Ligase, including GRAIL, GOLIATH and/or GODZILLA, and/or VHHs specific for a E3 Ubiquitin Ligase such as RNF133, RNF148, RNF128, RNF149, RNF130, RNF150, RNF122, RNF43, ZNRF3, ZNRF4, RNF13, RNF167, AMFR, STVN1, RNF170, RNF121, RNF175, RNF139, RNF145, MARCHF5, VFPL1, RNFT1, RNF180, RNF103, RNF182, RNF5, RNF185, RNF19A, RNF19B, RNF144A, RNF14413, RNF217, MARCHF1, MARCHF8, MARCHF2, MARCHF3, MARCHF11, MARCHF4, MARCHF9, MARCHF6, NR1H4, RNF126, DCST1, RNF152, RNF186, CGRRF1, MUL1, TRIM13, TRIM59, and/or RNF112.

TABLE 4

| | | SEQ |
|---|---|---|
| | | ID |
| Target | Sequence | NO |
| EGFR- | QVKLEESGGGSVQTGGSLRLTCAASGRTSRSYGMGWFRQAPGK | 29 |
| GRAIL | EREFVSGISWRGDSTGYADSVKGRFTISRDNAKNTVDLQMNSLK | |
| | PEDTAIYYCAAAAGSAWYGTLYEYDYWGQGTQVTVSSLEVLFQ | |
| | GPQVQLQESGGGLVQAGGSLRLSCAASGNISVQLDMGWYRQAP | |
| | GKEREFVAAINQGTTTYYADSVKGRFTISRDNAKNTVYLQMNSL | |
| | KPEDTAVYYCAVYLYDIWNHPYWGQGTQVTVSS | |
| | QVKLEESGGGSVQTGGSLRLTCAASGRTSRSYGMGWFRQAPGK | 30 |
| | EREFVSGISWRGDSTGYADSVKGRFTISRDNAKNTVDLQMNSLK | |
| | PEDTAIYYCAAAAGSAWYGTLYEYDYWGQGTQVTVSSLEVLFQ | |
| | GPQVQLQESGGGLVQAGGSLRLSCAASGSISGGKGMGWYRQAP | |
| | GKEREFVAAIGSGAITYYADSVKGRFTISRDNAKNTVYLQMNSL | |
| | KPEDTAVYYCAVYTTALDEYPYWGQGTQVTVSS | |
| | VQLVESGGGLVQAGGSLRLSCAASGRTFSSYAMGWFRQAPGKE | 31 |
| | REFVVAINWSSGSTYYADSVKGRFTISRDNAKNTMYLQMNSLKP | |
| | EDTAVYYCAAGYQINSGNYNFKDYEYDYWGQGTQVTVSSLEVL | |
| | FQGPQVQLQESGGGLVQAGGSLRLSCAASGSISGGKGMGWYRQ | |
| | APGKEREFVAAIGSGAITYYADSVKGRFTISRDNAKNTVYLQMNS | |
| | LKPEDTAVYYCAVYTTALDEYPYWGQGTQVTVSS | |
| | EVQLVESGGGLVQAGGSLRLSCAASGRTFSSYAMGWFRQAPGK | 32 |
| | EREFVVAINWSSGSTYYADSVKGRFTISRDNAKNTMYLQMNSLK | |
| | PEDTAVYYCAAGYQINSGNYNFKDYEYDYWGQGTQVTVSSLEV | |
| | LFQGPQVQLQESGGGLVQAGGSLRLSCAASGSISGGKGMGWYR | |
| | QAPGKEREFVAAIGSGAITYYADSVKGRFTISRDNAKNTVYLQM | |
| | NSLKPEDTAVYYCAVYTTALDEYPYWGQGTQVTVSS | |
| GRAIL- | QVQLQESGGGLVQAGGSLRLSCAASGNISVQLDMGWYRQAPGK | 33 |
| EGFR | EREFVAAINQGTTTYYADSVKGRFTISRDNAKNTVYLQMNSLKP | |
| | EDTAVYYCAVYLYDIWNHPYWGQGTQVTVSSLEVLFQGPQVKL | |
| | EESGGGSVQTGGSLRLTCAASGRTSRSYGMGWFRQAPGKEREFV | |
| | SGISWRGDSTGYADSVKGRFTISRDNAKNTVDLQMNSLKPEDTAI | |
| | YYCAAAAGSAWYGTLYEYDYWGQGTQVTVSS | |
| | QVQLQESGGGLVQAGGSLRLSCAASGSISGGKGMGWYRQAPGK | 34 |
| | EREFVAAIGSGAITYYADSVKGRFTISRDNAKNTVYLQMNSLKPE | |
| | DTAVYYCAVYTTALDEYPYWGQGTQVTVSSLEVLFQGPQVKLE | |
| | ESGGGSVQTGGSLRLTCAASGRTSRSYGMGWFRQAPGKEREFVS | |
| | GISWRGDSTGYADSVKGRFTISRDNAKNTVDLQMNSLKPEDTAI | |
| | YYCAAAAGSAWYGTLYEYDYWGQGTQVTVSS | |
| | QVQLQESGGGLVQAGGSLRLSCAASGSISGGKGMGWYRQAPGK | 35 |
| | EREFVAAIGSGAITYYADSVKGRFTISRDNAKNTVYLQMNSLKPE | |
| | DTAVYYCAVYTTALDEYPYWGQGTQVTVSSLEVLFQGPEVQLV | |
| | ESGGGLVQAGGSLRLSCAASGRTFSSYAMGWFRQAPGKEREFVV | |
| | AINWSSGSTYYADSVKGRFTISRDNAKNTMYLQMNSLKPEDTAV | |
| | YYCAAGYQINSGNYNFKDYEYDYWGQGTQVTVSS | |
| | QVQLQESGGGLVQAGGSLRLSCAASGSISGGKGMGWYRQAPGK | 36 |
| | EREFVAAIGSGAITYYADSVKGRFTISRDNAKNTVYLQMNSLKPE | |
| | DTAVYYCAVYTTALDEYPYWGQGTQVTVSSLEVLFQGPEVQLV | |
| | ESGGGLVQAGGSLRLSCAASGRTFSSYAMGWFRQAPGKEREFVV | |
| | AINWSSGSTYYADSVKGRFTISRDNAKNTMYLQMNSLKPEDTAV | |
| | YYCAAGYQINSGNYNFKDYEYDYWGQGTQVTVSS | |

Table 5 depicts the amino acid sequences of exemplary REULR constructs specific for the ECD of the E3 Ub ligase GOLIATH (RNF130) and the ECD of the PD-1 receptor. Two single variable domain heavy chain antibodies are connected with the 3C linker LEVLFQGP (SEQ ID NO: 73) that is designated by the underlined portion. In the example represented by SEQ ID NO: 37, the amino acid sequence of SEQ ID NO: 15 is present on the N-terminal side of the 3C linker and the amino acid sequence of SEQ ID NO: 6 is present on the C-terminal side of the 3C linker. In the example represented by SEQ ID NO: 38, the amino acid sequence of SEQ ID NO: 15 is present on the N-terminal side of the 3C linker and the amino acid sequence of SEQ ID NO: 5 is present on the C-terminal side of the 3C linker. In the example represented by SEQ ID NO: 39, the amino acid sequence of SEQ ID NO: 5 is present on the N-terminal side of the 3C linker and the amino acid sequence of SEQ ID NO: 15 is present on the C-terminal side of the 3C linker. In the example represented by SEQ ID NO: 40, the amino acid sequence of SEQ ID NO: 6 is present on the N-terminal side of the 3C linker and the amino acid sequence of SEQ ID NO: 15 is present on the C-terminal side of the 3C linker. While Table 5 depicts the presence of the 3C linker of SEQ ID NO: 73, the 3C linker of SEQ ID NO: 106 may be provided in replacement of the 3C linker of SEQ ID NO: 73.

It is specially contemplated that the specifically exemplified REULR constructs are exemplary only. The exemplified polypeptide sequence embodying the VHH to the target protein may have alterations in sequence with the proviso that such alterations do not remove its specificity for the target protein, while variability in terms of increased binding affinity, and reduced binding affinity while maintaining specificity, are contemplated. Other and different linkers/linker molecules are contemplated and, in that regard, the specific examples are not intended to be limiting. Further, other VHHs specific for other E3 Ubiquitin Ligases may be substituted, for example, VHHs specific for a RING type E3 Ubiquitin Ligase, including GRAIL, GOLIATH and/or GODZILLA, and/or VHHs specific for a E3 Ubiquitin Ligase such as RNF133, RNF148, RNF128, RNF149, RNF130, RNF150, RNF122, RNF43, ZNRF3, ZNRF4, RNF13, RNF167, AMFR, STVN1, RNF170, RNF121, RNF175, RNF139, RNF145, MARCHF5, VFPL1, RNFT1, RNF180, RNF103, RNF182, RNF5, RNF185, RNF19A, RNF19B, RNF144A, RNF144B, RNF217, MARCHF1, MARCHF8, MARCHF2, MARCHF3, MARCHF11, MARCHF4, MARCHF9, MARCHF6, NR1H4, RNF126, DCST1, RNF152, RNF186, CGRRF1, MUL1, TRIM13, TRIM59, and/or RNF112.

TABLE 5

| | | SEQ |
|---|---|---|
| | | ID |
| Target | Sequence | NO |
| PD-1- | DVQLVESGGGVVQPGGSLRLSCAASGSIASIHAMGWFRQAPGKE | 37 |
| GOLIATH | REFVAVITWSGGITYYADSVKGRFTISRDNSKNTVYLQMNSLRPE | |
| | DTALYYCAGDKHQSSWYDYWGQGTLVTVSSLEVLFQGPQVQLQ | |
| | ESGGGLVQAGGSLRLSCAASGTISFIGYMGWYRQAPGKERELVA | |
| | SIASGTSTYYADSVKGRFTISRDNAKNTVYLQMNSLKPEDTAVY | |
| | YCAATQYIQDVHRYWGQGTQVTVSS | |
| | DVQLVESGGGVVQPGGSLRLSCAASGSIASIHAMGWFRQAPGKE | 38 |
| | REFVAVITWSGGITYYADSVKGRFTISRDNSKNTVYLQMNSLRPE | |
| | DTALYYCAGDKHQSSWYDYWGQGTLVTVSSLEVLFQGPQVQLQ | |
| | ESGGGLVQAGGSLRLSCAASGYISGYYVMGWYRQAPGKEREFV | |
| | ASISYGASTYYADSVKGRFTISRDNAKNTVYLQMNSLKPEDTAV | |
| | YYCAVDFDSNYAHTYWGQGTQVTVSS | |
| GOLIATH- | QVQLQESGGGLVQAGGSLRLSCAASGYISGYYVMGWYRQAPGK | 39 |
| PD-1 | EREFVASISYGASTYYADSVKGRFTISRDNAKNTVYLQMNSLKPE | |
| | DTAVYYCAVDFDSNYAHTYWGQGTQVTVSSLEVLFQGPDVQLV | |
| | ESGGGVVQPGGSLRLSCAASGSIASIHAMGWFRQAPGKEREFVA | |
| | VITWSGGITYYADSVKGRFTISRDNSKNTVYLQMNSLRPEDTALY | |
| | YCAGDKHQSSWYDYWGQGTLVTVSS | |
| | QVQLQESGGGLVQAGGSLRLSCAASGTISFIGYMGWYRQAPGKE | 40 |
| | RELVASIASGTSTYYADSVKGRFTISRDNAKNTVYLQMNSLKPED | |
| | TAVYYCAATQYIQDVHRYWGQGTQVTVSSLEVLFQGPDVQLVE | |
| | SGGGVVQPGGSLRLSCAASGSIASIHAMGWFRQAPGKEREFVAVI | |
| | TWSGGITYYADSVKGRFTISRDNSKNTVYLQMNSLRPEDTALYY | |
| | CAGDKHQSSWYDYWGQGTLVTVSS | |

Table 6 depicts the amino acid sequences of exemplary REULR constructs specific for the ECD of the E3 Ub ligase GOLIATH (RNF130) and the ECD of the EGFR receptor. Two single variable domain heavy chain antibodies are connected with the 3C linker LEVLFQGP (SEQ ID NO: 73) that is designated by the underlined portion. In the example represented by SEQ ID NO: 41, the amino acid sequence of SEQ ID NO: 16 is present on the N-terminal side of the 3C linker and the amino acid sequence of SEQ ID NO: 5 is present on the C-terminal side of the 3C linker. In the example represented by SEQ ID NO: 42, the amino acid sequence of SEQ ID NO: 16 is present on the N-terminal side of the 3C linker and the amino acid sequence of SEQ ID NO: 6 is present on the C-terminal side of the 3C linker. In the example represented by SEQ ID NO: 43, the amino acid sequence of SEQ ID NO: 17 is present on the N-terminal side of the 3C linker and the amino acid sequence of SEQ ID NO: 5 is present on the C-terminal side of the 3C linker. In the example represented by SEQ ID NO: 44, the amino acid sequence of SEQ ID NO: 17 is present on the N-terminal side of the 3C linker and the amino acid sequence of SEQ ID NO: 6 is present on the C-terminal side of the 3C linker. In the example represented by SEQ ID NO: 45, the amino acid sequence of SEQ ID NO: 5 is present on the N-terminal side of the 3C linker and the amino acid sequence of SEQ ID NO: 16 is present on the C-terminal side of the 3C linker. In the example represented by SEQ ID NO: 46, the amino acid sequence of SEQ ID NO: 6 is present on the N-terminal side of the 3C linker and the amino acid sequence of SEQ ID NO: 16 is present on the C-terminal side of the 3C linker. In the example represented by SEQ ID NO: 47, the amino acid sequence of SEQ ID NO: 5 is present on the N-terminal side of the 3C linker and the amino acid sequence of SEQ ID NO: 17 is present on the C-terminal side of the 3C linker. In the example represented by SEQ ID NO: 48, the amino acid sequence of SEQ ID NO: 6 is present on the N-terminal side of the 3C linker and the amino acid sequence of SEQ ID NO: 17 is present on the C-terminal side of the 3C linker. While Table 6 depicts the presence of the 3C linker of SEQ ID NO: 73, the 3C linker of SEQ ID NO: 106 may be provided in replacement of the 3C linker of SEQ ID NO: 73.

It is specially contemplated that the specifically exemplified REULR constructs are exemplary only. The exemplified polypeptide sequence embodying the VHH to the target protein may have alterations in sequence with the proviso that such alterations do not remove its specificity for the target protein, while variability in terms of increased binding affinity, and reduced binding affinity while maintaining specificity, are contemplated. Other and different linkers/linker molecules are contemplated and, in that regard, the specific examples are not intended to be limiting. Further, other VHHs specific for other E3 Ubiquitin Ligases may be substituted, for example, VHHs specific for a RING type E3 Ubiquitin Ligase, including GRAIL, GOLIATH and/or GODZILLA, and/or VHHs specific for a E3 Ubiquitin Ligase such as RNF133, RNF148, RNF128, RNF149, RNF130, RNF150, RNF122, RNF43, ZNRF3, ZNRF4, RNF13, RNF167, AMFR, STVN1, RNF170, RNF121, RNF175, RNF139, RNF145, MARCHF5, VFPL1, RNFT1, RNF180, RNF103, RNF182, RNF5, RNF185, RNF19A, RNF19B, RNF144A, RNF144B, RNF217, MARCHF1, MARCHF8, MARCHF2, MARCHF3, MARCHF11, MARCHF4, MARCHF9, MARCHF6, NR1H4, RNF126, DCST1, RNF152, RNF186, CGRRF1, MUL1, TRIM13, TRIM59, and/or RNF112.

TABLE 6

| | | SEQ |
|---|---|---|
| | | ID |
| Target | Sequence | NO |
| EGFR- | QVKLEESGGGSVQTGGSLRLTCAASGRTSRSYGMGWFRQAPGK | 41 |
| GOLIATH | EREFVSGISWRGDSTGYADSVKGRFTISRDNAKNTVDLQMNSLK | |
| | PEDTAIYYCAAAAGSAWYGTLYEYDYWGQGTQVTVSSLEVLFQ | |
| | GPQVQLQESGGGLVQAGGSLRLSCAASGYISGYYVMGWYRQAP | |
| | GKEREFVASISYGASTYYADSVKGRFTISRDNAKNTVYLQMNSL | |
| | KPEDTAVYYCAVDFDSNYAHTYWGQGTQVTVSS | |
| | QVKLEESGGGSVQTGGSLRLTCAASGRTSRSYGMGWFRQAPGK | 42 |
| | EREFVSGISWRGDSTGYADSVKGRFTISRDNAKNTVDLQMNSLK | |
| | PEDTAIYYCAAAAGSAWYGTLYEYDYWGQGTQVTVSSLEVLFQ | |
| | GPQVQLQESGGGLVQAGGSLRLSCAASGTISFIGYMGWYRQAPG | |
| | KERELVASIASGTSTYYADSVKGRFTISRDNAKNTVYLQMNSLKP | |
| | EDTAVYYCAATQYIQDVHRYWGQGTQVTVSS | |
| | EVQLVESGGGLVQAGGSLRLSCAASGRTFSSYAMGWFRQAPGK | 43 |
| | EREFVVAINWSSGSTYYADSVKGRFTISRDNAKNTMYLQMNSLK | |
| | PEDTAVYYCAAGYQINSGNYNFKDYEYDYWGQGTQVTVSSLEV | |
| | LFQGPQVQLQESGGGLVQAGGSLRLSCAASGYISGYYVMGWYR | |
| | QAPGKEREFVASISYGASTYYADSVKGRFTISRDNAKNTVYLQM | |
| | NSLKPEDTAVYYCAVDFDSNYAHTYWGQGTQVTVSS | |
| | EVQLVESGGGLVQAGGSLRLSCAASGRTFSSYAMGWFRQAPGK | 44 |
| | EREFVVAINWSSGSTYYADSVKGRFTISRDNAKNTMYLQMNSLK | |
| | PEDTAVYYCAAGYQINSGNYNFKDYEYDYWGQGTQVTVSSLEV | |
| | LFQGPQVQLQESGGGLVQAGGSLRLSCAASGTISFIGYMGWYRQ | |
| | APGKERELVASIASGTSTYYADSVKGRFTISRDNAKNTVYLQMNS | |
| | LKPEDTAVYYCAATQYIQDVHRYWGQGTQVTVSS | |
| GOLIATH- | QVQLQESGGGLVQAGGSLRLSCAASGYISGYYVMGWYRQAPGK | 45 |
| EGFR | EREFVASISYGASTYYADSVKGRFTISRDNAKNTVYLQMNSLKPE | |
| | DTAVYYCAVDFDSNYAHTYWGQGTQVTVSSLEVLFQGPQVKLE | |
| | ESGGGSVQTGGSLRLTCAASGRTSRSYGMGWFRQAPGKEREFVS | |
| | GISWRGDSTGYADSVKGRFTISRDNAKNTVDLQMNSLKPEDTAI | |
| | YYCAAAAGSAWYGTLYEYDYWGQGTQVTVSS | |
| | QVQLQESGGGLVQAGGSLRLSCAASGTISFIGYMGWYRQAPGKE | 46 |
| | RELVASIASGTSTYYADSVKGRFTISRDNAKNTVYLQMNSLKPED | |
| | TAVYYCAATQYIQDVHRYWGQGTQVTVSSLEVLFQGPQVKLEE | |
| | SGGGSVQTGGSLRLTCAASGRTSRSYGMGWFRQAPGKEREFVSG | |
| | ISWRGDSTGYADSVKGRFTISRDNAKNTVDLQMNSLKPEDTAIY | |
| | YCAAAAGSAWYGTLYEYDYWGQGTQVTVSS | |
| | QVQLQESGGGLVQAGGSLRLSCAASGTISFIGYMGWYRQAPGKE | 47 |
| | RELVASIASGTSTYYADSVKGRFTISRDNAKNTVYLQMNSLKPED | |
| | TAVYYCAATQYIQDVHRYWGQGTQVTVSSLEVLFQGPEVQLVE | |
| | SGGGLVQAGGSLRLSCAASGRTFSSYAMGWFRQAPGKEREFVV | |
| | AINWSSGSTYYADSVKGRFTISRDNAKNTMYLQMNSLKPEDTAV | |
| | YYCAAGYQINSGNYNFKDYEYDYWGQGTQVTVSS | |
| | QVQLQESGGGLVQAGGSLRLSCAASGYISGYYVMGWYRQAPGK | 48 |
| | EREFVASISYGASTYYADSVKGRFTISRDNAKNTVYLQMNSLKPE | |
| | DTAVYYCAVDFDSNYAHTYWGQGTQVTVSSLEVLFQGPEVQLV | |
| | ESGGGLVQAGGSLRLSCAASGRTFSSYAMGWFRQAPGKEREFVV | |
| | AINWSSGSTYYADSVKGRFTISRDNAKNTMYLQMNSLKPEDTAV | |
| | YYCAAGYQINSGNYNFKDYEYDYWGQGTQVTVSS | |

Table 7 depicts the amino acid sequences of exemplary REULR constructs specific for the ECD of the E3 Ub ligase GRAIL (RNF128) and the ECD of the chemokine antagonist vMIP-II and the chemokine antagonist cMIP-II. Two single variable domain heavy chain antibodies are connected with the 3C linker LEVLFQGP (SEQ ID NO: 73) that is designated by the underlined portion. In the example represented by SEQ ID NO: 49, the amino acid sequence of SEQ ID NO: 18 is present on the N-terminal side of the 3C linker and the amino acid sequence of SEQ ID NO: 2 is present on the C-terminal side of the 3C linker. In the example represented by SEQ ID NO: 50, the amino acid sequence of SEQ ID NO: 18 is present on the N-terminal side of the 3C linker and the amino acid sequence of SEQ ID NO: 3 is present on the C-terminal side of the 3C linker. While Table 7 depicts the presence of the 3C linker of SEQ ID NO: 73, the 3C linker of SEQ ID NO: 106 may be provided in replacement of the 3C linker of SEQ ID NO: 73.

It is specially contemplated that the specifically exemplified REULR constructs are exemplary only. The exemplified polypeptide sequence embodying the VHH to the target protein may have alterations in sequence with the proviso that such alterations do not remove its specificity for the target protein, while variability in terms of increased binding affinity, and reduced binding affinity while maintaining specificity, are contemplated. Other and different linkers/linker molecules are contemplated and, in that regard, the specific examples are not intended to be limiting. Further, other VHHs specific for other E3 Ubiquitin Ligases may be substituted, for example, VHHs specific for a RING type E3 Ubiquitin Ligase, including GRAIL, GOLIATH and/or GODZILLA, and/or VHHs specific for a E3 Ubiquitin Ligase such as RNF133, RNF148, RNF128, RNF149, RNF130, RNF150, RNF122, RNF43, ZNRF3, ZNRF4, RNF13, RNF167, AMFR, STVN1, RNF170, RNF121, RNF175, RNF139, RNF145, MARCHF5, VFPL1, RNFT1, RNF180, RNF103, RNF182, RNF5, RNF185, RNF19A, RNF19B, RNF144A, RNF144B, RNF217, MARCHF1, MARCHF8, MARCHF2, MARCHF3, MARCHF11, MARCHF4, MARCHF9, MARCHF6, NR1H4, RNF126, DCST1, RNF152, RNF186, CGRRF1, MUL1, TRIM13, TRIM59, and/or RNF112.

TABLE 7

| | | SEQ |
|---|---|---|
| | | ID |
| Target | Sequence | NO |
| CXCR4/ | GDTLGASWHRPDKCCLGYQKRPLPQVLLSSWYPTSQLCSKPGVI | 49 |
| CCR1/ | FLTKRGRQVCADKSKDWVKKLMQQLPVTARLEVLFQGPQVQLQ | |
| CCR2/ | ESGGGLVQAGGSLRLSCAASGNISVQLDMGWYRQAPGKEREFV | |
| CCR5-- | AAINQGTTTYYADSVKGRFTISRDNAKNTVYLQMNSLKPEDTAV | |
| GRAIL | YYCAVYLYDIWNHPYWGQGTQVTVSS | |
| | GDTLGASWHRPDKCCLGYQKRPLPQVLLSSWYPTSQLCSKPGVI | 50 |
| | FLTKRGRQVCADKSKDWVKKLMQQLPVTARLEVLFQGPQVQLQ | |
| | ESGGGLVQAGGSLRLSCAASGSISGGKGMGWYRQAPGKEREFV | |
| | AAIGSGAITYYADSVKGRFTISRDNAKNTVYLQMNSLKPEDTAV | |
| | YYCAVYTTALDEYPYWGQGTQVTVSS | |

Table 8 depicts the amino acid sequences of exemplary REULR constructs specific for the ECD of the E3 Ub ligase GOLIATH (RNF130) and the ECD of the chemokine antagonist vMIP-II. Two single variable domain heavy chain antibodies are connected with the 3C linker LEVLFQGP (SEQ ID NO: 73) that is designated by the underlined portion. In the example represented by SEQ ID NO: 51, the amino acid sequence of SEQ ID NO: 18 is present on the N-terminal side of the 3C linker and the amino acid sequence of SEQ ID NO: 5 is present on the C-terminal side of the 3C linker. In the example represented by SEQ ID NO: 52, the amino acid sequence of SEQ ID NO: 18 is present on the N-terminal side of the 3C linker and the amino acid sequence of SEQ ID NO: 6 is present on the C-terminal side of the 3C linker. While Table 8 depicts the presence of the 3C linker of SEQ ID NO: 73, the 3C linker of SEQ ID NO: 106 may be provided in replacement of the 3C linker of SEQ ID NO: 73.

It is specially contemplated that the specifically exemplified REULR constructs are exemplary only. The exemplified polypeptide sequence embodying the VHH to the target protein may have alterations in sequence with the proviso that such alterations do not remove its specificity for the target protein, while variability in terms of increased binding affinity, and reduced binding affinity while maintaining specificity, are contemplated. Other and different linkers/linker molecules are contemplated and, in that regard, the specific examples are not intended to be limiting. Further, other VHHs specific for other E3 Ubiquitin Ligases may be substituted, for example, VHHs specific for a RING type E3 Ubiquitin Ligase, including GRAIL, GOLIATH and/or GODZILLA, and/or VHHs specific for a E3 Ubiquitin Ligase such as RNF133, RNF148, RNF128, RNF149, RNF130, RNF150, RNF122, RNF43, ZNRF3, ZNRF4, RNF13, RNF167, AMFR, STVN1, RNF170, RNF121, RNF175, RNF139, RNF145, MARCHF5, VFPL1, RNFT1, RNF180, RNF103, RNF182, RNF5, RNF185, RNF19A, RNF19B, RNF144A, RNF144B, RNF217, MARCHF1, MARCHF8, MARCHF2, MARCHF3, MARCHF11, MARCHF4, MARCHF9, MARCHF6, NR1H4, RNF126, DCST1, RNF152, RNF186, CGRRF1, MUL1, TRIM13, TRIM59, and/or RNF112.

TABLE 8

| | | SEQ |
|---|---|---|
| | | ID |
| Target | Sequence | NO |
| CXCR4/CCR | GDTLGASWHRPDKCCLGYQKRPLPQVLLSSWYPTSQLCSKPGVI | 51 |
| 1/CCR2/CCR | FLTKRGRQVCADKSKDWVKKLMQQLPVTARLEVLFQGPQVQLQ | |
| 5--GOLIATH | ESGGGLVQAGGSLRLSCAASGYISGYYVMGWYRQAPGKEREFV | |
| | ASISYGASTYYADSVKGRFTISRDNAKNTVYLQMNSLKPEDTAV | |
| | YYCAVDFDSNYAHTYWGQGTQVTVSS | |
| | GDTLGASWHRPDKCCLGYQKRPLPQVLLSSWYPTSQLCSKPGVI | 52 |
| | FLTKRGRQVCADKSKDWVKKLMQQLPVTARLEVLFQGPQVQLQ | |
| | ESGGGLVQAGGSLRLSCAASGTISFIGYMGWYRQAPGKERELVA | |
| | SIASGTSTYYADSVKGRFTISRDNAKNTVYLQMNSLKPEDTAVY | |
| | YCAATQYIQDVHRYWGQGTQVTVSS | |

Table 9 depicts the amino acid sequences of exemplary REULR constructs specific for the ECD of the E3 Ub ligase GOLIATH (RNF130) and the ECD of exemplary muscarinic acetylcholine receptor binders. See, e.g., Maeda et al., Science 369(6500):161-7 (2020). Two single variable domain heavy chain antibodies are connected with the 3C linker LEVLFQGP (SEQ ID NO: 73) that is designated by the underlined portion. In the example represented by SEQ ID NO: 53, the amino acid sequence of SEQ ID NO: 5 is present on the N-terminal side of the 3C linker and the amino acid sequence of SEQ ID NO: 19 is present on the C-terminal side of the 3C linker. In the example represented by SEQ ID NO: 54, the amino acid sequence of SEQ ID NO: 19 is present on the N-terminal side of the 3C linker and the amino acid sequence of SEQ ID NO: 5 is present on the C-terminal side of the 3C linker. In the example represented by SEQ ID NO: 55, the amino acid sequence of SEQ ID NO: 5 is present on the N-terminal side of the 3C linker and the amino acid sequence of SEQ ID NO: 20 is present on the C-terminal side of the 3C linker. In the example represented by SEQ ID NO: 56, the amino acid sequence of SEQ ID NO: 20 is present on the N-terminal side of the 3C linker and the amino acid sequence of SEQ ID NO: 5 is present on the C-terminal side of the 3C linker. While Table 9 depicts the presence of the 3C linker of SEQ ID NO: 73, the 3C linker of SEQ ID NO: 106 may be provided in replacement of the 3C linker of SEQ ID NO: 73.

It is specially contemplated that the specifically exemplified REULR constructs are exemplary only. The exemplified polypeptide sequence embodying the VHH to the target protein may have alterations in sequence with the proviso that such alterations do not remove its specificity for the target protein, while variability in terms of increased binding affinity, and reduced binding affinity while maintaining specificity, are contemplated. Other and different linkers/linker molecules are contemplated and, in that regard, the specific examples are not intended to be limiting. Further, other VHHs specific for other E3 Ubiquitin Ligases may be substituted, for example, VHHs specific for a RING type E3 Ubiquitin Ligase, including GRAIL, GOLIATH and/or GODZILLA, and/or VHHs specific for a E3 Ubiquitin Ligase such as RNF133, RNF148, RNF128, RNF149, RNF130, RNF150, RNF122, RNF43, ZNRF3, ZNRF4, RNF13, RNF167, AMFR, STVN1, RNF170, RNF121, RNF175, RNF139, RNF145, MARCHF5, VFPL1, RNFT1, RNF180, RNF103, RNF182, RNF5, RNF185, RNF19A, RNF19B, RNF144A, RNF144B, RNF217, MARCHF1, MARCHF8, MARCHF2, MARCHF3, MARCHF11, MARCHF4, MARCHF9, MARCHF6, NR1H4, RNF126, DCST1, RNF152, RNF186, CGRRF1, MUL1, TRIM13, TRIM59, and/or RNF112.

TABLE 9

| | | SEQ |
|---|---|---|
| | | ID |
| Target | Sequence | NO |
| GOLIATH- | QVQLQESGGGLVQAGGSLRLSCAASGYISGYYVMGWYRQAPGK | 53 |
| M1AChR | EREFVASISYGASTYYADSVKGRFTISRDNAKNTVYLQMNSLKPE | |
| | DTAVYYCAVDFDSNYAHTYWGQGTQVTVSSLEVLFQGPLTCVK | |
| | SNSIWFPTSEDCPDGQNLCFKRWQYISPRMYDFTRGCAATCPKAE | |
| | YRDVINCCGTDKCNK | |
| M1AChR-- | LTCVKSNSIWFPTSEDCPDGQNLCFKRWQYISPRMYDFTRGCAAT | 54 |
| GOLIATH | CPKAEYRDVINCCGTDKCNKLEVLFQGPQVQLQESGGGLVQAG | |
| | GSLRLSCAASGYISGYYVMGWYRQAPGKEREFVASISYGASTYY | |
| | ADSVKGRFTISRDNAKNTVYLQMNSLKPEDTAVYYCAVDFDSN | |
| | YAHTYWGQGTQVTVSS | |
| GOLIATH-- | QVQLQESGGGLVQAGGSLRLSCAASGYISGYYVMGWYRQAPGK | 55 |
| M2AChR | EREFVASISYGASTYYADSVKGRFTISRDNAKNTVYLQMNSLKPE | |
| | DTAVYYCAVDFDSNYAHTYWGQGTQVTVSSLEVLFQGPLTCVK | |
| | SNSIRFPTSGDCPDGQNLCFKRWQSPGMPRPMWALVCAATCPKA | |
| | PPNEDINCCGTDKCNK | |
| M2AChR-- | LTCVKSNSIRFPTSGDCPDGQNLCFKRWQSPGMPRPMWALVCAA | 56 |
| GOLIATH | TCPKAPPNEDINCCGTDKCNKLEVLFQGPQVQLQESGGGLVQAG | |
| | GSLRLSCAASGYISGYYVMGWYRQAPGKEREFVASISYGASTYY | |
| | ADSVKGRFTISRDNAKNTVYLQMNSLKPEDTAVYYCAVDFDSN | |
| | YAHTYWGQGTQVTVSS | |

Table 10 depicts the amino acid sequences of exemplary REULR constructs specific for the ECD of the E3 Ub ligase GRAIL (RNF128) and the ECD of the mouse PD-1 receptor. Two single variable domain heavy chain antibodies are connected with the 3C linker LEVLFQGP (SEQ ID NO: 73) that is designated by the underlined portion. In the example represented by SEQ ID NO: 57, the amino acid sequence of SEQ ID NO: 2 is present on the N-terminal side of the 3C linker and the amino acid sequence of SEQ ID NO: 21 is present on the C-terminal side of the 3C linker. In the example represented by SEQ ID NO: 58, the amino acid sequence of SEQ ID NO: 3 is present on the N-terminal side of the 3C linker and the amino acid sequence of SEQ ID NO: 16 is present on the C-terminal side of the 3C linker. In the example represented by SEQ ID NO: 59, the amino acid sequence of SEQ ID NO: 21 is present on the N-terminal side of the 3C linker and the amino acid sequence of SEQ ID NO: 2 is present on the C-terminal side of the 3C linker. In the example represented by SEQ ID NO: 60, the amino acid sequence of SEQ ID NO: 21 is present on the N-terminal side of the 3C linker and the amino acid sequence of SEQ ID NO: 3 is present on the C-terminal side of the 3C linker. While Table 10 depicts the presence of the 3C linker of SEQ ID NO: 73, the 3C linker of SEQ ID NO: 106 may be provided in replacement of the 3C linker of SEQ ID NO: 73.

It is specially contemplated that the specifically exemplified REULR constructs are exemplary only. The exemplified polypeptide sequence embodying the VHH to the target protein may have alterations in sequence with the proviso that such alterations do not remove its specificity for the target protein, while variability in terms of increased binding affinity, and reduced binding affinity while maintaining specificity, are contemplated. Other and different linkers/linker molecules are contemplated and, in that regard, the specific examples are not intended to be limiting. Further, other VHHs specific for other E3 Ubiquitin Ligases may be substituted, for example, VHHs specific for a RING type E3 Ubiquitin Ligase, including GRAIL, GOLIATH and/or GODZILLA, and/or VHHs specific for a E3 Ubiquitin Ligase such as RNF133, RNF148, RNF128, RNF149, RNF130, RNF150, RNF122, RNF43, ZNRF3, ZNRF4, RNF13, RNF167, AMFR, STVN1, RNF170, RNF121, RNF175, RNF139, RNF145, MARCHF5, VFPL1, RNFT1, RNF180, RNF103, RNF182, RNF5, RNF185, RNF19A, RNF19B, RNF144A, RNF144B, RNF217, MARCHF1, MARCHF8, MARCHF2, MARCHF3, MARCHF11, MARCHF4, MARCHF9, MARCHF6, NR1H4, RNF126, DCST1, RNF152, RNF186, CGRRF1, MUL1, TRIM13, TRIM59, and/or RNF112.

TABLE 10

| | | SEQ |
|---|---|---|
| | | ID |
| Target | Sequence | NO |
| GRAIL- | QVQLQESGGGLVQAGGSLRLSCAASGNISVQLDMGWYRQAPGK | 57 |
| mPD-1 | EREFVAAINQGTTTYYADSVKGRFTISRDNAKNTVYLQMNSLKP | |
| | EDTAVYYCAVYLYDIWNHPYWGQGTQVTVSSGSLEVLFQGPGS | |
| | DIVLTQSTLPLPVSPGESVSITCRSSKSLLYSDGKTYLNWYLQRPG | |
| | QSPQLLIYWMSTRASGVSDRFSGSGSGTDFTLKISGVEAEDVGIY | |
| | YCQQGLEFPTFGGGTKLELKGGSTRSSSSGGGGSGGGGEVQLQES | |
| | GPGLVKPSQSLSLTCSVTGYSITSSYRWNWIRQFPGNRLEWMGYI | |
| | NSAGISNYNPSLKRRISITRDTSKNQFFLQVNSVTTEDTATYYCAR | |
| | SDNMGTTPFTYWGQGTLVTVSS | |
| | QVQLQESGGGLVQAGGSLRLSCAASGSISGGKGMGWYRQAPGK | 58 |
| | EREFVAAIGSGAITYYADSVKGRFTISRDNAKNTVYLQMNSLKPE | |
| | DTAVYYCAVYTTALDEYPYWGQGTQVTVSSGSLEVLFQGPGSDI | |
| | VLTQSTLPLPVSPGESVSITCRSSKSLLYSDGKTYLNWYLQRPGQS | |
| | PQLLIYWMSTRASGVSDRFSGSGSGTDFTLKISGVEAEDVGIYYC | |
| | QQGLEFPTFGGGTKLELKGGSTRSSSSGGGGSGGGGEVQLQESGP | |
| | GLVKPSQSLSLTCSVTGYSITSSYRWNWIRQFPGNRLEWMGYINS | |
| | AGISNYNPSLKRRISITRDTSKNQFFLQVNSVTTEDTATYYCARSD | |
| | NMGTTPFTYWGQGTLVTVSS | |
| mPD-1-- | DIVLTQSTLPLPVSPGESVSITCRSSKSLLYSDGKTYLNWYLQRPG | 59 |
| GRAIL | QSPQLLIYWMSTRASGVSDRFSGSGSGTDFTLKISGVEAEDVGIY | |
| | YCQQGLEFPTFGGGTKLELKGGSTRSSSSGGGGSGGGGEVQLQES | |
| | GPGLVKPSQSLSLTCSVTGYSITSSYRWNWIRQFPGNRLEWMGYI | |
| | NSAGISNYNPSLKRRISITRDTSKNQFFLQVNSVTTEDTATYYCA | |
| | RSDNMGTTPFTYWGQGTLVTVSSGSLEVLFQGPGSQVQLQESGGG | |
| | LVQAGGSLRLSCAASGNISVQLDMGWYRQAPGKEREFVAAINQG | |
| | TTTYYADSVKGRFTISRDNAKNTVYLQMNSLKPEDTAVYYCAV | |
| | YLYDIWNHPYWGQGTQVTVSS | |
| | DIVLTQSTLPLPVSPGESVSITCRSSKSLLYSDGKTYLNWYLQRPG | 60 |
| | QSPQLLIYWMSTRASGVSDRFSGSGSGTDFTLKISGVEAEDVGIY | |
| | YCQQGLEFPTFGGGTKLELKGGSTRSSSSGGGGSGGGGEVQLQES | |
| | GPGLVKPSQSLSLTCSVTGYSITSSYRWNWIRQFPGNRLEWMGYI | |
| | NSAGISNYNPSLKRRISITRDTSKNQFFLQVNSVTTEDTATYYCA | |
| | RSDNMGTTPFTYWGQGTLVTVSSGSLEVLFQGPGSQVQLQESGGG | |
| | LVQAGGSLRLSCAASGSISGGKGMGWYRQAPGKEREFVAAIGSG | |
| | AITYYADSVKGRFTISRDNAKNTVYLQMNSLKPEDTAVYYCAVY | |
| | TTALDEYPYWGQGTQVTVSS | |

Table 11 depicts the amino acid sequences of exemplary REULR constructs specific for the ECD of the E3 Ub ligase GOLIATH (RNF130) and the ECD of the mouse PD-1 receptor. Two single variable domain heavy chain antibodies are connected with the 3C linker LEVLFQGP (SEQ ID NO: 73) that is designated by the underlined portion. In the example represented by SEQ ID NO: 61, the amino acid sequence of SEQ ID NO: 5 is present on the N-terminal side of the 3C linker and the amino acid sequence of SEQ ID NO: 21 is present on the C-terminal side of the 3C linker. In the example represented by SEQ ID NO: 62, the amino acid sequence of SEQ ID NO: 6 is present on the N-terminal side of the 3C linker and the amino acid sequence of SEQ ID NO: 21 is present on the C-terminal side of the 3C linker. In the example represented by SEQ ID NO: 63, the amino acid sequence of SEQ ID NO: 21 is present on the N-terminal side of the 3C linker and the amino acid sequence of SEQ ID NO: 5 is present on the C-terminal side of the 3C linker. In the example represented by SEQ ID NO: 64, the amino acid sequence of SEQ ID NO: 21 is present on the N-terminal side of the 3C linker and the amino acid sequence of SEQ ID NO: 6 is present on the C-terminal side of the 3C linker. While Table 11 depicts the presence of the 3C linker of SEQ ID NO: 73, the 3C linker of SEQ ID NO: 106 may be provided in replacement of the 3C linker of SEQ ID NO: 73.

It is specially contemplated that the specifically exemplified REULR constructs are exemplary only. The exemplified polypeptide sequence embodying the VHH to the target protein may have alterations in sequence with the proviso that such alterations do not remove its specificity for the target protein, while variability in terms of increased binding affinity, and reduced binding affinity while maintaining specificity, are contemplated. Other and different linkers/linker molecules are contemplated and, in that regard, the specific examples are not intended to be limiting. Further, other VHHs specific for other E3 Ubiquitin Ligases may be substituted, for example, VHHs specific for a RING type E3 Ubiquitin Ligase, including GRAIL, GOLIATH and/or GODZILLA, and/or VHHs specific for a E3 Ubiquitin Ligase such as RNF133, RNF148, RNF128, RNF149, RNF130, RNF150, RNF122, RNF43, ZNRF3, ZNRF4, RNF13, RNF167, AMFR, STVN1, RNF170, RNF121, RNF175, RNF139, RNF145, MARCHF5, VFPL1, RNFT1, RNF180, RNF103, RNF182, RNF5, RNF185, RNF19A, RNF19B, RNF144A, RNF144B, RNF217, MARCHF1, MARCHF8, MARCHF2, MARCHF3, MARCHF11, MARCHF4, MARCHF9, MARCHF6, NR1H4, RNF126, DCST1, RNF152, RNF186, CGRRF1, MUL1, TRIM13, TRIM59, and/or RNF112.

TABLE 11

| | | SEQ |
|---|---|---|
| | | ID |
| Target | Sequence | NO |
| GOLIATH- | QVQLQESGGGLVQAGGSLRLSCAASGYISGYYVMGWYRQAPGK | 61 |
| mPD-1 | EREFVASISYGASTYYADSVKGRFTISRDNAKNTVYLQMNSLKPE | |
| | DTAVYYCAVDFDSNYAHTYWGQGTQVTVSSGSLEVLFQGPGSDI | |
| | VLTQSTLPLPVSPGESVSITCRSSKSLLYSDGKTYLNWYLQRPGQS | |
| | PQLLIYWMSTRASGVSDRFSGSGSGTDFTLKISGVEAEDVGIYYC | |
| | QQGLEFPTFGGGTKLELKGGSTRSSSSGGGGSGGGGEVQLQESGP | |
| | GLVKPSQSLSLTCSVTGYSITSSYRWNWIRQFPGNRLEWMGYINS | |
| | AGISNYNPSLKRRISITRDTSKNQFFLQVNSVTTEDTATYYCARSD | |
| | NMGTTPFTYWGQGTLVTVSS | |
| | QVQLQESGGGLVQAGGSLRLSCAASGTISFIGYMGWYRQAPGKE | 62 |
| | RELVASIASGTSTYYADSVKGRFTISRDNAKNTVYLQMNSLKPED | |
| | TAVYYCAATQYIQDVHRYWGQGTQVTVSSGSLEVLFQGPGSDIV | |
| | LTQSTLPLPVSPGESVSITCRSSKSLLYSDGKTYLNWYLQRPGQSP | |
| | QLLIYWMSTRASGVSDRFSGSGSGTDFTLKISGVEAEDVGIYYCQ | |
| | QGLEFPTFGGGTKLELKGGSTRSSSSGGGGSGGGGEVQLQESGPG | |
| | LVKPSQSLSLTCSVTGYSITSSYRWNWIRQFPGNRLEWMGYINSA | |
| | GISNYNPSLKRRISITRDTSKNQFFLQVNSVTTEDTATYYCARSDN | |
| | MGTTPFTYWGQGTLVTVSS | |
| mPD-1-- | DIVLTQSTLPLPVSPGESVSITCRSSKSLLYSDGKTYLNWYLQRPG | 63 |
| GOLIATH | QSPQLLIYWMSTRASGVSDRFSGSGSGTDFTLKISGVEAEDVGIY | |
| | YCQQGLEFPTFGGGTKLELKGGSTRSSSSGGGGSGGGGEVQLQES | |
| | GPGLVKPSQSLSLTCSVTGYSITSSYRWNWIRQFPGNRLEWMGYI | |
| | NSAGISNYNPSLKRRISITRDTSKNQFFLQVNSVTTEDTATYYCAR | |
| | SDNMGTTPFTYWGQGTLVTVSSGSLEVLFQGPGSQVQLQESGGG | |
| | LVQAGGSLRLSCAASGYISGYYVMGWYRQAPGKEREFVASISYG | |
| | ASTYYADSVKGRFTISRDNAKNTVYLQMNSLKPEDTAVYYCAV | |
| | DFDSNYAHTYWGQGTQVTVSS | |
| | DIVLTQSTLPLPVSPGESVSITCRSSKSLLYSDGKTYLNWYLQRPG | 64 |
| | QSPQLLIYWMSTRASGVSDRFSGSGSGTDFTLKISGVEAEDVGIY | |
| | YCQQGLEFPTFGGGTKLELKGGSTRSSSSGGGGSGGGGEVQLQES | |
| | GPGLVKPSQSLSLTCSVTGYSITSSYRWNWIRQFPGNRLEWMGYI | |
| | NSAGISNYNPSLKRRISITRDTSKNQFFLQVNSVTTEDTATYYCAR | |
| | SDNMGTTPFTYWGQGTLVTVSSGSLEVLFQGPGSQVQLQESGGG | |
| | LVQAGGSLRLSCAASGTISFIGYMGWYRQAPGKERELVASIASGT | |
| | STYYADSVKGRFTISRDNAKNTVYLQMNSLKPEDTAVYYCAATQ | |
| | YIQDVHRYWGQGTQVTVSS | |

Table 12 depicts the amino acid sequences of exemplary REULR constructs specific for the ECD of the E3 Ub ligase GRAIL (RNF128) and the ECD of the mouse EGFR receptor. Two single variable domain heavy chain antibodies are connected with the 3C linker LEVLFQGP (SEQ ID NO: 73) that is designated by the underlined portion. In the example represented by SEQ ID NO: 65, the amino acid sequence of SEQ ID NO: 22 is present on the N-terminal side of the 3C linker and the amino acid sequence of SEQ ID NO: 2 is present on the C-terminal side of the 3C linker. In the example represented by SEQ ID NO: 66, the amino acid sequence of SEQ ID NO: 22 is present on the N-terminal side of the 3C linker and the amino acid sequence of SEQ ID NO: 3 is present on the C-terminal side of the 3C linker. In the example represented by SEQ ID NO: 67, the amino acid sequence of SEQ ID NO: 2 is present on the N-terminal side of the 3C linker and the amino acid sequence of SEQ ID NO: 22 is present on the C-terminal side of the 3C linker. In the example represented by SEQ ID NO: 68, the amino acid sequence of SEQ ID NO: 3 is present on the N-terminal side of the 3C linker and the amino acid sequence of SEQ ID NO: 22 is present on the C-terminal side of the 3C linker. While Table 12 depicts the presence of the 3C linker of SEQ ID NO: 73, the 3C linker of SEQ ID NO: 106 may be provided in replacement of the 3C linker of SEQ ID NO: 73.

It is specially contemplated that the specifically exemplified REULR constructs are exemplary only. The exemplified polypeptide sequence embodying the VHH to the target protein may have alterations in sequence with the proviso that such alterations do not remove its specificity for the target protein, while variability in terms of increased binding affinity, and reduced binding affinity while maintaining specificity, are contemplated. Other and different linkers/linker molecules are contemplated and, in that regard, the specific examples are not intended to be limiting. Further, other VHHs specific for other E3 Ubiquitin Ligases may be substituted, for example, VHHs specific for a RING type E3 Ubiquitin Ligase, including GRAIL, GOLIATH and/or GODZILLA, and/or VHHs specific for a E3 Ubiquitin Ligase such as RNF133, RNF148, RNF128, RNF149, RNF130, RNF150, RNF122, RNF43, ZNRF3, ZNRF4, RNF13, RNF167, AMFR, STVN1, RNF170, RNF121, RNF175, RNF139, RNF145, MARCHF5, VFPL1, RNFT1, RNF180, RNF103, RNF182, RNF5, RNF185, RNF19A, RNF19B, RNF144A, RNF14413, RNF217, MARCHF1, MARCHF8, MARCHF2, MARCHF3, MARCHF11, MARCHF4, MARCHF9, MARCHF6, NR1H4, RNF126, DCST1, RNF152, RNF186, CGRiRFI, MUL1, TRIM13, TRIM59, and/or RNFl12.

TABLE 12

| | | SEQ |
|---|---|---|
| | | ID |
| Target | Sequence | NO |
| mEGFR-- | EVMLVESGGVLVKPGGSLKLSCAASGFTFSRYAMSWVRQTPEKR | 65 |
| GRAIL | LEWVATISSGGSYSYYPDSVKGRFTISRDNVKNTLYLQMSSLRSE | |
| | DTAMYYCARDSGGFAYWGQWGQGTLVTVSAGGGGSGGGGSG | |
| | GGGSDIQMTQTTSSLSASLGDRVTISCRASQDISNYLNWYQQKPD | |
| | GTVKLLIYYTSRLHSGVTSRFSGSGSGTDYSLTISNLEQEDIATYFC | |
| | QQGNTLPWTFGGGTKKVEIKRADLEVLFQGPQVQLQESGGGLVQ | |
| | AGGSLRLSCAASGTISFIGYMGWYRQAPGKERELVASIASGTSTY | |
| | YADSVKGRFTISRDNAKNTVYLQMNSLKPEDTAVYYCAATQYIQ | |
| | DVHRYWGQGTQVTVSS | |
| | EVMLVESGGVLVKPGGSLKLSCAASGFTFSRYAMSWVRQTPEKR | 66 |
| | LEWVATISSGGSYSYYPDSVKGRFTISRDNVKNTLYLQMSSLRSE | |
| | DTAMYYCARDSGGFAYWGQWGQGTLVTVSAGGGGSGGGGSG | |
| | GGGSDIQMTQTTSSLSASLGDRVTISCRASQDISNYLNWYQQKPD | |
| | GTVKLLIYYTSRLHSGVTSRFSGSGSGTDYSLTISNLEQEDIATYFC | |
| | QQGNTLPWTFGGGTKKVEIKRADLEVLFQGPQVQLQESGGGLVQ | |
| | AGGSLRLSCAASGSISGGKGMGWYRQAPGKEREFVAAIGSGAIT | |
| | YYADSVKGRFTISRDNAKNTVYLQMNSLKPEDTAVYYCAVYTT | |
| | ALDEYPYWGQGTQVTVSS | |
| GRAIL-- | QVQLQESGGGLVQAGGSLRLSCAASGTISFIGYMGWYRQAPGKE | 67 |
| mEGFR | RELVASIASGTSTYYADSVKGRFTISRDNAKNTVYLQMNSLKPED | |
| | TAVYYCAATQYIQDVHRYWGQGTQVTVSSLEVLFQGPEVMLVE | |
| | SGGVLVKPGGSLKLSCAASGFTFSRYAMSWVRQTPEKRLEWVAT | |
| | ISSGGSYSYYPDSVKGRFTISRDNVKNTLYLQMSSLRSEDTAMYY | |
| | CARDSGGFAYWGQWGQGTLVTVSAGGGGSGGGGSGGGGSDIQ | |
| | MTQTTSSLSASLGDRVTISCRASQDISNYLNWYQQKPDGTVKLLI | |
| | YYTSRLHSGVTSRFSGSGSGTDYSLTISNLEQEDIATYFCQQGNTL | |
| | PWTFGGGTKKVEIKRAD | |
| | QVQLQESGGGLVQAGGSLRLSCAASGSISGGKGMGWYRQAPGK | 68 |
| | EREFVAAIGSGAITYYADSVKGRFTISRDNAKNTVYLQMNSLKPE | |
| | DTAVYYCAVYTTALDEYPYWGQGTQVTVSSLEVLFQGPEVMLV | |
| | ESGGVLVKPGGSLKLSCAASGFTFSRYAMSWVRQTPEKRLEWVA | |
| | TISSGGSYSYYPDSVKGRFTISRDNVKNTLYLQMSSLRSEDTAMY | |
| | YCARDSGGFAYWGQWGQGTLVTVSAGGGGSGGGGSGGGGSDI | |
| | QMTQTTSSLSASLGDRVTISCRASQDISNYLNWYQQKPDGTVKLL | |
| | IYYTSRLHSGVTSRFSGSGSGTDYSLTISNLEQEDIATYFCQQGNT | |
| | LPWTFGGGTKKVEIKRAD | |

Table 13 depicts the amino acid sequences of exemplary REULR constructs specific for the ECD of the E3 Ub ligase GOLIATH (RNF130) and the ECD of the mouse EGFR receptor. Two single variable domain heavy chain antibodies are connected with the 3C linker LEVLFQGP (SEQ ID NO: 73) that is designated by the underlined portion. In the example represented by SEQ ID NO: 69, the amino acid sequence of SEQ ID NO: 22 is present on the N-terminal side of the 3C linker and the amino acid sequence of SEQ ID NO: 5 is present on the C-terminal side of the 3C linker. In the example represented by SEQ ID NO: 70, the amino acid sequence of SEQ ID NO: 22 is present on the N-terminal side of the 3C linker and the amino acid sequence of SEQ ID NO: 6 is present on the C-terminal side of the 3C linker. In the example represented by SEQ ID NO: 71, the amino acid sequence of SEQ ID NO: 5 is present on the N-terminal side of the 3C linker and the amino acid sequence of SEQ ID NO: 22 is present on the C-terminal side of the 3C linker. In the example represented by SEQ ID NO: 73, the amino acid sequence of SEQ ID NO: 6 is present on the N-terminal side of the 3C linker and the amino acid sequence of SEQ ID NO: 22 is present on the C-terminal side of the 3C linker. While Table 13 depicts the presence of the 3C linker of SEQ ID NO: 73, the 3C linker of SEQ ID NO: 106 may be provided in replacement of the 3C linker of SEQ ID NO: 73.

It is specially contemplated that the specifically exemplified REULR constructs are exemplary only. The exemplified polypeptide sequence embodying the VHH to the target protein may have alterations in sequence with the proviso that such alterations do not remove its specificity for the target protein, while variability in terms of increased binding affinity, and reduced binding affinity while maintaining specificity, are contemplated. Other and different linkers/linker molecules are contemplated and, in that regard, the specific examples are not intended to be limiting. Further, other VHHs specific for other E3 Ubiquitin Ligases may be substituted, for example, VHHs specific for a RING type E3 Ubiquitin Ligase, including GRAIL, GOLIATH and/or GODZILLA, and/or VHHs specific for a E3 Ubiquitin Ligase such as RNF133, RNF148, RNF128, RNF149, RNF130, RNF150, RNF122, RNF43, ZNRF3, ZNRF4, RNF13, RNF167, AMFR, STVN1, RNF170, RNF121, RNF175, RNF139, RNF145, MARCHF5, VFPL1, RNFT1, RNF180, RNF103, RNF182, RNF5, RNF185, RNF19A, RNF19B, RNF144A, RNF144B, RNF217, MARCHF1, MARCHF8, MARCHF2, MARCHF3, MARCHF11, MARCHF4, MARCHF9, MARCHF6, NR1H4, RNF126, DCST1, RNF152, RNF186, CGRRF1, MUL1, TRIM13, TRIM59, and/or RNF112.

TABLE 13

| | | SEQ |
|---|---|---|
| | | ID |
| Target | Sequence | NO |
| mEGFR-- | EVMLVESGGVLVKPGGSLKLSCAASGFTFSRYAMSWVRQTPEKR | 69 |
| GOLIATH | LEWVATISSGGSYSYYPDSVKGRFTISRDNVKNTLYLQMSSLRSE | |
| | DTAMYYCARDSGGFAYWGQWGQGTLVTVSAGGGGSGGGGSG | |
| | GGGSDIQMTQTTSSLSASLGDRVTISCRASQDISNYLNWYQQKPD | |
| | GTVKLLIYYTSRLHSGVTSRFSGSGSGTDYSLTISNLEQEDIATYFC | |
| | QQGNTLPWTFGGGTKKVEIKRADLEVLFQGPQVQLQESGGGLVQ | |
| | AGGSLRLSCAASGYISGYYVMGWYRQAPGKEREFVASISYGAST | |
| | YYADSVKGRFTISRDNAKNTVYLQMNSLKPEDTAVYYCAVDFD | |
| | SNYAHTYWGQGTQVTVSS | |
| | EVMLVESGGVLVKPGGSLKLSCAASGFTFSRYAMSWVRQTPEKR | 70 |
| | LEWVATISSGGSYSYYPDSVKGRFTISRDNVKNTLYLQMSSLRSE | |
| | DTAMYYCARDSGGFAYWGQWGQGTLVTVSAGGGGSGGGGSG | |
| | GGGSDIQMTQTTSSLSASLGDRVTISCRASQDISNYLNWYQQKPD | |
| | GTVKLLIYYTSRLHSGVTSRFSGSGSGTDYSLTISNLEQEDIATYFC | |
| | QQGNTLPWTFGGGTKKVEIKRADLEVLFQGPQVQLQESGGGLVQ | |
| | AGGSLRLSCAASGTISFIGYMGWYRQAPGKERELVASIASGTSTY | |
| | YADSVKGRFTISRDNAKNTVYLQMNSLKPEDTAVYYCAATQYIQ | |
| | DVHRYWGQGTQVTVSS | |
| GOLIATH-- | QVQLQESGGGLVQAGGSLRLSCAASGYISGYYVMGWYRQAPGK | 71 |
| mEGFR | EREFVASISYGASTYYADSVKGRFTISRDNAKNTVYLQMNSLKPE | |
| | DTAVYYCAVDFDSNYAHTYWGQGTQVTVSSLEVLFQGPEVMLV | |
| | ESGGVLVKPGGSLKLSCAASGFTFSRYAMSWVRQTPEKRLEWVA | |
| | TISSGGSYSYYPDSVKGRFTISRDNVKNTLYLQMSSLRSEDTAMY | |
| | YCARDSGGFAYWGQWGQGTLVTVSAGGGGSGGGGSGGGGSDI | |
| | QMTQTTSSLSASLGDRVTISCRASQDISNYLNWYQQKPDGTVKLL | |
| | IYYTSRLHSGVTSRFSGSGSGTDYSLTISNLEQEDIATYFCQQGNT | |
| | LPWTFGGGTKKVEIKRAD | |
| | QVQLQESGGGLVQAGGSLRLSCAASGTISFIGYMGWYRQAPGKE | 72 |
| | RELVASIASGTSTYYADSVKGRFTISRDNAKNTVYLQMNSLKPED | |
| | TAVYYCAATQYIQDVHRYWGQGTQVTVSSLEVLFQGPEVMLVE | |
| | SGGVLVKPGGSLKLSCAASGFTFSRYAMSWVRQTPEKRLEWVAT | |
| | ISSGGSYSYYPDSVKGRFTISRDNVKNTLYLQMSSLRSEDTAMYY | |
| | CARDSGGFAYWGQWGQGTLVTVSAGGGGSGGGGSGGGGSDIQ | |
| | MTQTTSSLSASLGDRVTISCRASQDISNYLNWYQQKPDGTVKLLI | |
| | YYTSRLHSGVTSRFSGSGSGTDYSLTISNLEQEDIATYFCQQGNTL | |
| | PWTFGGGTKKVEIKRAD | |

Table 14 depicts the amino acid sequences of exemplary REULR constructs specific for the ECD of the E3 Ub ligase GRAIL, GOLIATH, and GODZILLA and the ECD of the erythropoietin receptor (DA10). The polypeptide sequence of SEQ ID NO: 74 represents an exemplary VHH specific for the erythropoietin receptor (DA10). According to the contemplated REULR constructs, two single variable domain heavy chain antibodies (VHHs/nanobodies) are connected with the 3C linker LEVLFQGP (SEQ ID NO: 73) that is designated by the underlined portion. In the example represented by SEQ ID NO: 75, the amino acid sequence of SEQ ID NO: 74 is present on the N-terminal side of the 3C linker and the amino acid sequence of SEQ ID NO: 2 is present on the C-terminal side of the 3C linker. In the example represented by SEQ ID NO: 76, the amino acid sequence of SEQ ID NO: 2 is present on the N-terminal side of the 3C linker and the amino acid sequence of SEQ ID NO: 74 is present on the C-terminal side of the 3C linker. In the example represented by SEQ ID NO: 77, the amino acid sequence of SEQ ID NO: 74 is present on the N-terminal side of the 3C linker and the amino acid sequence of SEQ ID NO: 3 is present on the C-terminal side of the 3C linker. In the example represented by SEQ ID NO: 78, the amino acid sequence of SEQ ID NO: 3 is present on the N-terminal side of the 3C linker and the amino acid sequence of SEQ ID NO: 74 is present on the C-terminal side of the 3C linker. In the example represented by SEQ ID NO: 79, the amino acid sequence of SEQ ID NO: 74 is present on the N-terminal side of the 3C linker and the amino acid sequence of SEQ ID NO: 5 is present on the C-terminal side of the 3C linker. In the example represented by SEQ ID NO: 80, the amino acid sequence of SEQ ID NO: 5 is present on the N-terminal side of the 3C linker and the amino acid sequence of SEQ ID NO: 74 is present on the C-terminal side of the 3C linker. In the example represented by SEQ ID NO: 81, the amino acid sequence of SEQ ID NO: 74 is present on the N-terminal side of the 3C linker and the amino acid sequence of SEQ ID NO: 6 is present on the C-terminal side of the 3C linker. In the example represented by SEQ ID NO: 82, the amino acid sequence of SEQ ID NO: 6 is present on the N-terminal side of the 3C linker and the amino acid sequence of SEQ ID NO: 74 is present on the C-terminal side of the 3C linker. In the example represented by SEQ ID NO: 83, the amino acid sequence of SEQ ID NO: 74 is present on the N-terminal side of the 3C linker and the amino acid sequence of SEQ ID NO: 9 is present on the C-terminal side of the 3C linker. In the example represented by SEQ ID NO: 84, the amino acid sequence of SEQ ID NO: 9 is present on the N-terminal side of the 3C linker and the amino acid sequence of SEQ ID NO: 74 is present on the C-terminal side of the 3C linker. While Table 14 depicts the presence of the 3C linker of SEQ ID NO: 73, the 3C linker of SEQ ID NO: 106 may be provided in replacement of the 3C linker of SEQ ID NO: 73.

It is specially contemplated that the specifically exemplified REULR constructs are exemplary only. The exemplified polypeptide sequence embodying the VHH to the target protein may have alterations in sequence with the proviso that such alterations do not remove its specificity for the target protein, while variability in terms of increased binding affinity, and reduced binding affinity while maintaining specificity, are contemplated. Other and different linkers/linker molecules are contemplated and, in that regard, the specific examples are not intended to be limiting. Further, other VHHs specific for other E3 Ubiquitin Ligases may be substituted, for example, VHHs specific for another RING type E3 Ubiquitin Ligase, including GRAIL, GOLIATH and/or GODZILLA, and/or VHHs specific for a E3 Ubiquitin Ligase such as RNF133, RNF148, RNF128, RNF149, RNF130, RNF150, RNF122, RNF43, ZNRF3, ZNRF4, RNF13, RNF167, AMFR, STVN1, RNF170, RNF121, RNF175, RNF139, RNF145, MARCHF5, VFPL1, RNFT1, RNF180, RNF103, RNF182, RNF5, RNF185, RNF19A, RNF19B, RNF144A, RNF144B, RNF217, MARCHF1, MARCHF8, MARCHF2, MARCHF3, MARCHF11, MARCHF4, MARCHF9, MARCHF6, NR1H4, RNF126, DCST1, RNF152, RNF186, CGRRF1, MUL1, TRIM13, TRIM59, and/or RNF112.

TABLE 14

| | | SEQ |
|---|---|---|
| | | ID |
| Target | Sequence | NO |
| EPOR-DA10 | EVQLLESGGGLVQPGGSLRLSCAASGFTFSSYAMSWVRQAPGKGL | 74 |
| | EWVSAISGSGGSTYYADSVKGRFTISRDNSKNTLYLQMNSLRAED | |
| | TAVYYCVKDRVAVAGKGSYYFDSWGRGTTVTVSSGGGGSGGGG | |
| | SGGGGSQSVLTQPPSVSEAPGQRVTIACSGSSSNIGNNAVSWYQQL | |
| | PGKAPTLLIYYDNLLPSGVSDRFSGSKSGTSASLAISGLQSEDEADY | |
| | YCAAWDDSLNDWVFGGGTKVTVL | |
| EPOR- | EVQLLESGGGLVQPGGSLRLSCAASGFTFSSYAMSWVRQAPGKGL | 75 |
| DA10- | EWVSAISGSGGSTYYADSVKGRFTISRDNSKNTLYLQMNSLRAED | |
| GRAIL-E1 | TAVYYCVKDRVAVAGKGSYYFDSWGRGTTVTVSSGGGGSGGGG | |
| | SGGGGSQSVLTQPPSVSEAPGQRVTIACSGSSSNIGNNAVSWYQQL | |
| | PGKAPTLLIYYDNLLPSGVSDRFSGSKSGTSASLAISGLQSEDEADY | |
| | YCAAWDDSLNDWVFGGGTKVTVLGSLEVLFQGPGSQVQLQESG | |
| | GGLVQAGGSLRLSCAASGNISVQLDMGWYRQAPGKEREFVAAIN | |
| | QGTTTYYADSVKGRFTISRDNAKNTVYLQMNSLKPEDTAVYYCA | |
| | VYLYDIWNHPYWGQGTQVTVSS | |
| GRAIL-E1- | QVQLQESGGGLVQAGGSLRLSCAASGNISVQLDMGWYRQAPGKE | 76 |
| EPOR-DA10 | REFVAAINQGTTTYYADSVKGRFTISRDNAKNTVYLQMNSLKPED | |
| | TAVYYCAVYLYDIWNHPYWGQGTQVTVSSGSLEVLFQGPGSEVQ | |
| | LLESGGGLVQPGGSLRLSCAASGFTFSSYAMSWVRQAPGKGLEW | |
| | VSAISGSGGSTYYADSVKGRFTISRDNSKNTLYLQMNSLRAEDTA | |
| | VYYCVKDRVAVAGKGSYYFDSWGRGTTVTVSSGGGGSGGGGSG | |
| | GGGSQSVLTQPPSVSEAPGQRVTIACSGSSSNIGNNAVSWYQQLPG | |
| | KAPTLLIYYDNLLPSGVSDRFSGSKSGTSASLAISGLQSEDEADYYC | |
| | AAWDDSLNDWVFGGGTKVTVL | |
| EPOR- | EVQLLESGGGLVQPGGSLRLSCAASGFTFSSYAMSWVRQAPGKGL | 77 |
| DA10- | EWVSAISGSGGSTYYADSVKGRFTISRDNSKNTLYLQMNSLRAED | |
| GRAIL-E2 | TAVYYCVKDRVAVAGKGSYYFDSWGRGTTVTVSSGGGGSGGGG | |
| | SGGGGSQSVLTQPPSVSEAPGQRVTIACSGSSSNIGNNAVSWYQQL | |
| | PGKAPTLLIYYDNLLPSGVSDRFSGSKSGTSASLAISGLQSEDEADY | |
| | YCAAWDDSLNDWVFGGGTKVTVLGSLEVLFQGPGSQVQLQESG | |
| | GGLVQAGGSLRLSCAASGSISGGKGMGWYRQAPGKEREFVAAIG | |
| | SGAITYYADSVKGRFTISRDNAKNTVYLQMNSLKPEDTAVYYCAV | |
| | YTTALDEYPYWGQGTQVTVSS | |
| GRAIL-E2-- | QVQLQESGGGLVQAGGSLRLSCAASGSISGGKGMGWYRQAPGKE | 78 |
| EPOR-DA10 | REFVAAIGSGAITYYADSVKGRFTISRDNAKNTVYLQMNSLKPED | |
| | TAVYYCAVYTTALDEYPYWGQGTQVTVSSGSLEVLFQGPGSEVQ | |
| | LLESGGGLVQPGGSLRLSCAASGFTFSSYAMSWVRQAPGKGLEW | |
| | VSAISGSGGSTYYADSVKGRFTISRDNSKNTLYLQMNSLRAEDTA | |
| | VYYCVKDRVAVAGKGSYYFDSWGRGTTVTVSSGGGGSGGGGSG | |
| | GGGSQSVLTQPPSVSEAPGQRVTIACSGSSSNIGNNAVSWYQQLPG | |
| | KAPTLLIYYDNLLPSGVSDRFSGSKSGTSASLAISGLQSEDEADYYC | |
| | AAWDDSLNDWVFGGGTKVTVL | |
| EPOR- | TAVYYCVKDRVAVAGKGSYYFDSWGRGTTVTVSSGGGGSGGGG | 79 |
| DA10- | EVQLLESGGGLVQPGGSLRLSCAASGFTFSSYAMSWVRQAPGKGL | |
| GOLIATH-A1 | EWVSAISGSGGSTYYADSVKGRFTISRDNSKNTLYLQMNSLRAED | |
| | SGGGGSQSVLTQPPSVSEAPGQRVTIACSGSSSNIGNNAVSWYQQL | |
| | PGKAPTLLIYYDNLLPSGVSDRFSGSKSGTSASLAISGLQSEDEADY | |
| | YCAAWDDSLNDWVFGGGTKVTVLGSLEVLFQGPGSQVQLQESG | |
| | GGLVQAGGSLRLSCAASGYISGYYVMGWYRQAPGKEREFVASIS | |
| | YGASTYYADSVKGRFTISRDNAKNTVYLQMNSLKPEDTAVYYCA | |
| | VDFDSNYAHTYWGQGTQVTVSS | |
| GOLIATH- | QVQLQESGGGLVQAGGSLRLSCAASGYISGYYVMGWYRQAPGKE | 80 |
| A1-EPOR- | REFVASISYGASTYYADSVKGRFTISRDNAKNTVYLQMNSLKPED | |
| DA10 | TAVYYCAVDFDSNYAHTYWGQGTQVTVSSGSLEVLFQGPGSEVQ | |
| | LLESGGGLVQPGGSLRLSCAASGFTFSSYAMSWVRQAPGKGLEW | |
| | VSAISGSGGSTYYADSVKGRFTISRDNSKNTLYLQMNSLRAEDTA | |
| | VYYCVKDRVAVAGKGSYYFDSWGRGTTVTVSSGGGGSGGGGSG | |
| | GGGSQSVLTQPPSVSEAPGQRVTIACSGSSSNIGNNAVSWYQQLPG | |
| | KAPTLLIYYDNLLPSGVSDRFSGSKSGTSASLAISGLQSEDEADYYC | |
| | AAWDDSLNDWVFGGGTKVTVL | |
| EPOR- | EVQLLESGGGLVQPGGSLRLSCAASGFTFSSYAMSWVRQAPGKGL | 81 |
| DA10- | EWVSAISGSGGSTYYADSVKGRFTISRDNSKNTLYLQMNSLRAED | |
| GOLIATH-D1 | TAVYYCVKDRVAVAGKGSYYFDSWGRGTTVTVSSGGGGSGGGG | |
| | SGGGGSQSVLTQPPSVSEAPGQRVTIACSGSSSNIGNNAVSWYQQL | |
| | PGKAPTLLIYYDNLLPSGVSDRFSGSKSGTSASLAISGLQSEDEADY | |
| | YCAAWDDSLNDWVFGGGTKVTVLGSLEVLFQGPGSQVQLQESG | |
| | GGLVQAGGSLRLSCAASGTISFIGYMGWYRQAPGKERELVASIAS | |
| | GTSTYYADSVKGRFTISRDNAKNTVYLQMNSLKPEDTAVYYCAA | |
| | TQYIQDVHRYWGQGTQVTVSS | |
| GOLIATH- | QVQLQESGGGLVQAGGSLRLSCAASGTISFIGYMGWYRQAPGKER | 82 |
| D1-EPOR- | ELVASIASGTSTYYADSVKGRFTISRDNAKNTVYLQMNSLKPEDT | |
| DA10 | AVYYCAATQYIQDVHRYWGQGTQVTVSSGSLEVLFQGPGSEVQL | |
| | LESGGGLVQPGGSLRLSCAASGFTFSSYAMSWVRQAPGKGLEWV | |
| | SAISGSGGSTYYADSVKGRFTISRDNSKNTLYLQMNSLRAEDTAV | |
| | YYCVKDRVAVAGKGSYYFDSWGRGTTVTVSSGGGGSGGGGSGG | |
| | GGSQSVLTQPPSVSEAPGQRVTIACSGSSSNIGNNAVSWYQQLPGK | |
| | APTLLIYYDNLLPSGVSDRFSGSKSGTSASLAISGLQSEDEADYYCA | |
| | AWDDSLNDWVFGGGTKVTVL | |
| EPOR- | EVQLLESGGGLVQPGGSLRLSCAASGFTFSSYAMSWVRQAPGKGL | 83 |
| DA10- | EWVSAISGSGGSTYYADSVKGRFTISRDNSKNTLYLQMNSLRAED | |
| GODZILLA- | TAVYYCVKDRVAVAGKGSYYFDSWGRGTTVTVSSGGGGSGGGG | |
| A5 | SGGGGSQSVLTQPPSVSEAPGQRVTIACSGSSSNIGNNAVSWYQQL | |
| | PGKAPTLLIYYDNLLPSGVSDRFSGSKSGTSASLAISGLQSEDEADY | |
| | YCAAWDDSLNDWVFGGGTKVTVLGSLEVLFQGPGSQVQLQESG | |
| | GGLVQAGGSLRLSCAASGSIFRLWYMGWYRQAPGKEREFVASIGI | |
| | GATTNYADSVKGRFTISRDNAKNTVYLQMNSLKPEDTAVYYCAV | |
| | FGWAYSGYHDDFLYWGQGTQVTVSS | |
| GODZILLA- | QVQLQESGGGLVQAGGSLRLSCAASGSIFRLWYMGWYRQAPGKE | 84 |
| A5-EPOR- | REFVASIGIGATTNYADSVKGRFTISRDNAKNTVYLQMNSLKPEDT | |
| DA10 | AVYYCAVFGWAYSGYHDDFLYWGQGTQVTVSSGSLEVLFQGPG | |
| | SEVQLLESGGGLVQPGGSLRLSCAASGFTFSSYAMSWVRQAPGKG | |
| | LEWVSAISGSGGSTYYADSVKGRFTISRDNSKNTLYLQMNSLRAE | |
| | DTAVYYCVKDRVAVAGKGSYYFDSWGRGTTVTVSSGGGGSGGG | |
| | GSGGGGSQSVLTQPPSVSEAPGQRVTIACSGSSSNIGNNAVSWYQQ | |
| | LPGKAPTLLIYYDNLLPSGVSDRFSGSKSGTSASLAISGLQSEDEAD | |
| | YYCAAWDDSLNDWVFGGGTKVTVL | |

Table 15 depicts the amino acid sequences of exemplary REULR constructs specific for the ECD of the E3 Ub ligase GODZILLA and the ECDs of PD1, EGFR (7d12 and 9g8), and murine PD1. The polypeptide sequence of SEQ ID NO: 85 represents an exemplary VHH specific for PD1. The polypeptide sequence of SEQ ID NO: 86 represents an exemplary VHH specific for EGFR (7d12). The polypeptide sequence of SEQ ID NO: 87 represents a VHH specific for EGFR (9g8). The polypeptide sequence of SEQ ID NO: 85 represents an exemplary VHH specific for murine PD1. According to the contemplated REULR constructs, two single variable domain heavy chain antibodies (VHHs/nanobodies) are connected with the 3C linker LEVLFQGP (SEQ ID NO: 73) that is designated by the underlined portion. In the example represented by SEQ ID NO: 89, the amino acid sequence of SEQ ID NO: 85 is present on the N-terminal side of the 3C linker and the amino acid sequence of SEQ ID NO: 9 is present on the C-terminal side of the 3C linker. In the example represented by SEQ ID NO: 90, the amino acid sequence of SEQ ID NO: 9 is present on the N-terminal side of the 3C linker and the amino acid sequence of SEQ ID NO: 85 is present on the C-terminal side of the 3C linker. In the example represented by SEQ ID NO: 91, the amino acid sequence of SEQ ID NO: 87 is present on the N-terminal side of the 3C linker and the amino acid sequence of SEQ ID NO: 9 is present on the C-terminal side of the 3C linker. In the example represented by SEQ ID NO: 92, the amino acid sequence of SEQ ID NO: 9 is present on the N-terminal side of the 3C linker and the amino acid sequence of SEQ ID NO: 87 is present on the C-terminal side of the 3C linker. In the example represented by SEQ ID NO: 93, the amino acid sequence of SEQ ID NO: 88 is present on the N-terminal side of the 3C linker and the amino acid sequence of SEQ ID NO: 9 is present on the C-terminal side of the 3C linker. In the example represented by SEQ ID NO: 94, the amino acid sequence of SEQ ID NO: 9 is present on the N-terminal side of the 3C linker and the amino acid sequence of SEQ ID NO: 88 is present on the C-terminal side of the 3C linker. In the example represented by SEQ ID NO: 95, the amino acid sequence of SEQ ID NO: 86 is present on the N-terminal side of the 3C linker and the amino acid sequence of SEQ ID NO: 9 is present on the C-terminal side of the 3C linker. In the example represented by SEQ ID NO: 96, the amino acid sequence of SEQ ID NO: 9 is present on the N-terminal side of the 3C linker and the amino acid sequence of SEQ ID NO: 86 is present on the C-terminal side of the 3C linker. While Table 15 depicts the presence of the 3C linker of SEQ ID NO: 73, the 3C linker of SEQ ID NO: 106 may be provided in replacement of the 3C linker of SEQ ID NO: 73.

It is specially contemplated that the specifically exemplified REULR constructs are exemplary only. The exemplified polypeptide sequence embodying the VHH to the target protein may have alterations in sequence with the proviso that such alterations do not remove its specificity for the target protein, while variability in terms of increased binding affinity, and reduced binding affinity while maintaining specificity, are contemplated. Other and different linkers/linker molecules are contemplated and, in that regard, the specific examples are not intended to be limiting. Further, other VHHs specific for other E3 Ubiquitin Ligases may be substituted, for example, VHHs specific for a RING type E3 Ubiquitin Ligase, including GRAIL, GOLIATH and/or GODZILLA, and/or VHHs specific for a E3 Ubiquitin Ligase such as RNF133, RNF148, RNF128, RNF149, RNF130, RNF150, RNF122, RNF43, ZNRF3, ZNRF4, RNF13, RNF167, AMFR, STVN1, RNF170, RNF121, RNF175, RNF139, RNF145, MARCHF5, VFPL1, RNFT1, RNF180, RNF103, RNF182, RNF5, RNF185, RNF19A, RNF19B, RNF144A, RNF14413, RNF217, MARCHF1, MARCHF8, MARCHF2, MARCHF3, MARCHF11, MARCHF4, MARCHF9, MARCHF6, NR1H4, RNF126, DCST1, RNF152, RNF186, CGRiRFI, MUL1, TRIM13, TRIM59, and/or RNF112.

TABLE 15

| | | SEQ |
|---|---|---|
| | | ID |
| Target | Sequence | NO |
| PD1 | DVQLVESGGGVVQPGGSLRLSCAASGSIASIHAMGWFRQAPG | 85 |
| | KEREFVAVITWSGGITYYADSVKGRFTISRDNSKNTVYLQMNS | |
| | LRPEDTALYYCAGDKHQSSWYDYWGQGTLVTVSS | |
| mPD1 | DIVLTQSTLPLPVSPGESVSITCRSSKSLLYSDGKTYLNWYLQR | 86 |
| | PGQSPQLLIYWMSTRASGVSDRFSGSGSGTDFTLKISGVEAED | |
| | VGIYYCQQGLEFPTFGGGTKLELKGGSTRSSSSGGGGSGGGGE | |
| | VQLQESGPGLVKPSQSLSLTCSVTGYSITSSYRWNWIRQFPGN | |
| | RLEWMGYINSAGISNYNPSLKRRISITRDTSKNQFFLQVNSVTT | |
| | EDTATYYCARSDNMGTTPFTYWGQGTLVTVSS | |
| EGFR-7d12 | QVKLEESGGGSVQTGGSLRLTCAASGRTSRSYGMGWFRQAPG | 87 |
| | KEREFVSGISWRGDSTGYADSVKGRFTISRDNAKNTVDLQMN | |
| | SLKPEDTAIYYCAAAAGSAWYGTLYEYDYWGQGTQVTVSS | |
| EGFR-9g8 | EVQLVESGGGLVQAGGSLRLSCAASGRTFSSYAMGWFRQAPG | 88 |
| | KEREFVVAINWSSGSTYYADSVKGRFTISRDNAKNTMYLQMN | |
| | SLKPEDTAVYYCAAGYQINSGNYNFKDYEYDYWGQGTQVTV | |
| | SS | |
| PD1- | DVQLVESGGGVVQPGGSLRLSCAASGSIASIHAMGWFRQAPG | 89 |
| GODZILLA- | KEREFVAVITWSGGITYYADSVKGRFTISRDNSKNTVYLQMNS | |
| A5 | LRPEDTALYYCAGDKHQSSWYDYWGQGTLVTVSSLEVLFQG | |
| | PQVQLQESGGGLVQAGGSLRLSCAASGSIFRLWYMGWYRQAP | |
| | GKEREFVASIGIGATTNYADSVKGRFTISRDNAKNTVYLQMNS | |
| | LKPEDTAVYYCAVFGWAYSGYHDDFLYWGQGTQVTVSS | |
| GODZILLA- | QVQLQESGGGLVQAGGSLRLSCAASGSIFRLWYMGWYRQAP | 90 |
| A5-PD1 | GKEREFVASIGIGATTNYADSVKGRFTISRDNAKNTVYLQMNS | |
| | LKPEDTAVYYCAVFGWAYSGYHDDFLYWGQGTQVTVSSLEV | |
| | LFQGPDVQLVESGGGVVQPGGSLRLSCAASGSIASIHAMGWFR | |
| | QAPGKEREFVAVITWSGGITYYADSVKGRFTISRDNSKNTVYL | |
| | QMNSLRPEDTALYYCAGDKHQSSWYDYWGQGTLVTVSS | |
| EGFR-7d12 | QVKLEESGGGSVQTGGSLRLTCAASGRTSRSYGMGWFRQAPG | 91 |
| GODZILLA- | KEREFVSGISWRGDSTGYADSVKGRFTISRDNAKNTVDLQMN | |
| A5 | SLKPEDTAIYYCAAAAGSAWYGTLYEYDYWGQGTQVTVSSL | |
| | EVLFQGPQVQLQESGGGLVQAGGSLRLSCAASGSIFRLWYMG | |
| | WYRQAPGKEREFVASIGIGATTNYADSVKGRFTISRDNAKNTV | |
| | YLQMNSLKPEDTAVYYCAVFGWAYSGYHDDFLYWGQGTQV | |
| | TVSS | |
| GODZILLA- | QVQLQESGGGLVQAGGSLRLSCAASGSIFRLWYMGWYRQAP | 92 |
| A5-EGFR- | GKEREFVASIGIGATTNYADSVKGRFTISRDNAKNTVYLQMNS | |
| 7d12 | LKPEDTAVYYCAVFGWAYSGYHDDFLYWGQGTQVTVSSLEV | |
| | LFQGPQVKLEESGGGSVQTGGSLRLTCAASGRTSRSYGMGWF | |
| | RQAPGKEREFVSGISWRGDSTGYADSVKGRFTISRDNAKNTVD | |
| | LQMNSLKPEDTAIYYCAAAAGSAWYGTLYEYDYWGQGTQVT | |
| | VSS | |
| EGFR-9g8- | EVQLVESGGGLVQAGGSLRLSCAASGRTFSSYAMGWFRQAPG | 93 |
| GODZILLA- | KEREFVVAINWSSGSTYYADSVKGRFTISRDNAKNTMYLQMN | |
| A5 | SLKPEDTAVYYCAAGYQINSGNYNFKDYEYDYWGQGTQVTV | |
| | SSLEVLFQGPQVQLQESGGGLVQAGGSLRLSCAASGSIFRLWY | |
| | MGWYRQAPGKEREFVASIGIGATTNYADSVKGRFTISRDNAK | |
| | NTVYLQMNSLKPEDTAVYYCAVFGWAYSGYHDDFLYWGQG | |
| | TQVTVSS | |
| GODZILLA- | QVQLQESGGGLVQAGGSLRLSCAASGSIFRLWYMGWYRQAP | 94 |
| A5-EGFR- | GKEREFVASIGIGATTNYADSVKGRFTISRDNAKNTVYLQMNS | |
| 9g8 | LKPEDTAVYYCAVFGWAYSGYHDDFLYWGQGTQVTVSSLEV | |
| | LFQGPEVQLVESGGGLVQAGGSLRLSCAASGRTFSSYAMGWF | |
| | RQAPGKEREFVVAINWSSGSTYYADSVKGRFTISRDNAKNTM | |
| | YLQMNSLKPEDTAVYYCAAGYQINSGNYNFKDYEYDYWGQG | |
| | TQVTVSS | |
| mPD1-RMP | DIVLTQSTLPLPVSPGESVSITCRSSKSLLYSDGKTYLNWYLQR | 95 |
| GODZILLA- | PGQSPQLLIYWMSTRASGVSDRFSGSGSGTDFTLKISGVEAED | |
| A5 | VGIYYCQQGLEFPTFGGGTKLELKGGSTRSSSSGGGGSGGGGE | |
| | VQLQESGPGLVKPSQSLSLTCSVTGYSITSSYRWNWIRQFPGN | |
| | RLEWMGYINSAGISNYNPSLKRRISITRDTSKNQFFLQVNSVTT | |
| | EDTATYYCARSDNMGTTPFTYWGQGTLVTVSSGSLEVLFQGP | |
| | GSQVQLQESGGGLVQAGGSLRLSCAASGSIFRLWYMGWYRQ | |
| | APGKEREFVASIGIGATTNYADSVKGRFTISRDNAKNTVYLQM | |
| | NSLKPEDTAVYYCAVFGWAYSGYHDDFLYWGQGTQVTVSS | |
| GODZILLA- | QVQLQESGGGLVQAGGSLRLSCAASGSIFRLWYMGWYRQAP | 96 |
| A5-mPD1- | GKEREFVASIGIGATTNYADSVKGRFTISRDNAKNTVYLQMNS | |
| RMP | LKPEDTAVYYCAVFGWAYSGYHDDFLYWGQGTQVTVSSGSL | |
| | EVLFQGPGSDIVLTQSTLPLPVSPGESVSITCRSSKSLLYSDGKT | |
| | YLNWYLQRPGQSPQLLIYWMSTRASGVSDRFSGSGSGTDFTL | |
| | KISGVEAEDVGIYYCQQGLEFPTFGGGTKLELKGGSTRSSSSGG | |
| | GGSGGGGEVQLQESGPGLVKPSQSLSLTCSVTGYSITSSYRWN | |
| | WIRQFPGNRLEWMGYINSAGISNYNPSLKRRISITRDTSKNQFF | |
| | LQVNSVTTEDTATYYCARSDNMGTTPFTYWGQGTLVTVSS | |

### Example 1

HEK293F cells were transiently transfected with myc-tagged full length GRAIL cDNA (hRNF128; human) and FLAG-tagged full length PD-1 cDNA (human). 48h post transfection, transfected cells were either incubated with PD1-GRAIL Nanobody versions as indicated (PD1-E1, PD1-E2, E1-PD1, E2-PD1; 0.5 μM) or with 3C protease cleaved PD1-GRAIL versions for 6h (37° C.; 5% CO2) in the presence of Cycloheximide. Cells were subsequently washed (3×) before FACS analysis using FLAG-Tag (D6W5B) Rabbit mAb (Alexa Fluor 647 Conjugate). Data are mean+s.d. from n=3 replicates.

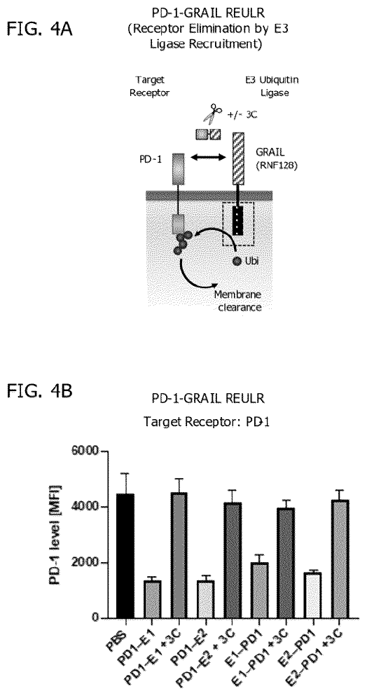

FIG. 4 depicts targeted degradation PD-1 by GRAIL-PD1 REULR molecule as measured by reduction in cell surface PD1 levels. The REULR molecules were engineered with a 3C enzyme cleavage site that functions as the linker between the two binding modules between substrate and E3 Ligase. Addition of 3C protease cleaves the REULR molecule into two pieces and activity is lost, thereby showing targeting E3 Ub ligase to alternative targets. As such these results show PD-1 receptor modulation by E3 Ubiquitin Ligase Recruitment using exemplary REULR constructs of the present disclosure specific for the ECD of the E3 ligase GRAIL and ECD of PD-1, with and without cleavage with 3C protease. The PD1-GRAIL REULR molecules that were cleaved with 3C protease remain inactive due to loss of the bispecific nature of the REULR activity and as a consequence show no effect on PD-1 surface levels.

### Example 2

HEK293F cells were transiently transfected with myc-tagged full length GRAIL cDNA (hRNF128; human) and FLAG-tagged full length PD-1 cDNA (human). 48h post transfection, transfected cells were either incubated with PD1-GRAIL Nanobody versions alone (PD1-E1, PD1-E2, E1-PD1, E2-PD1; 0.25 μM) or precincubated with monomeric GRAIL Nanobody (E1, E2; 40× excess) for 0.5h as indicated. Cells were incubated for 6h (37° C.; 5% CO2) in the presence of Cycloheximide and subsequently washed (3×) before FACS analysis using FLAG-Tag (D6W5B) Rabbit mAb (Alexa Fluor 647 Conjugate). Data are mean+s.d. from n=3 replicates.

FIG. 5 depicts PD-1 receptor modulation by E3 Ubiquitin Ligase Recruitment using exemplary REULR constructs of the present disclosure specific for the ECD of the E3 ligase GRAIL and ECD of PD-1 in the presence and absence of a blocking nanobody. FIG. 5A provides a schematic model of a Receptor PD1-GRAIL REULR concept with and without pre-incubation of excess (e.g., 40×) monomeric GRAIL VHH. Enforced GRAIL E3 ubiquitin Ligase recruitment to PD-1 reduces PD-1 cell surface levels by ubiquitination and subsequent membrane clearance by using intact REULR in comparison to GRAIL neutralized with excess monomeric GRAIL VHH. FIG. 4B depicts results of enforced recruitment of GRAIL to PD-1 using different version of PD-1-GRAIL REULR molecules reduces PD-1 cell surface levels when treated with intact PD-1-GRAIL REULR. To validate the specificity of PD-1 surface loss suing the PD-1-GRAIL REULR, the GRAIL receptor was blocked with excess of monomeric GRAIL VHH before adding the intact PD-1-GRAIL REULR and as a consequence rendered the addition of PD-1-GRAIL REULR molecule inert, neutralizing the PD-1-GRAIL REULR activity

### Example 3

HEK293F cells were transiently transfected with myc-tagged full length GOLIATH cDNA (hRNF130; human) and FLAG-tagged full length PD-1 cDNA (human). 48h post transfection, transfected cells were either incubated with PD1-GOLIATH Nanobody versions as indicated (A1-PD 1, D1-PD1, PD1-D1; 0.5 μM) or with 3C protease cleaved PD1-GOLIATH versions for 6h (37° C.; 5% CO2) in the presence of Cycloheximide. Cells were subsequently washed (3×) before FACS analysis using FLAG-Tag (D6W5B) Rabbit mAb (Alexa Fluor 647 Conjugate). Data are mean+s.d. from n=3 replicates.

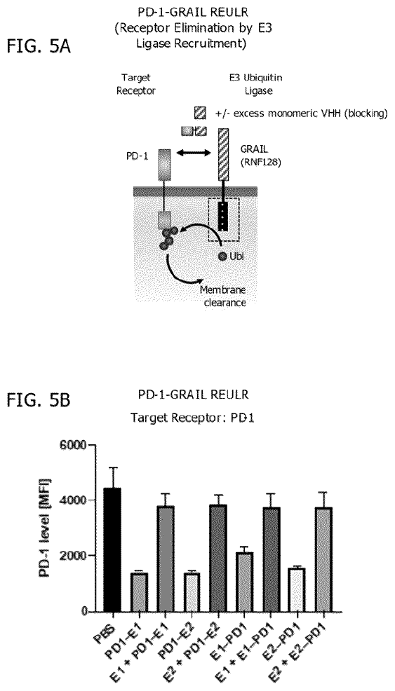

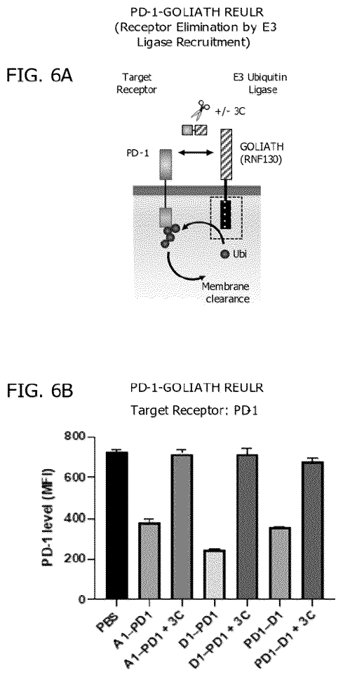

FIG. 6 depicts PD-1 receptor elimination by PD-1-GOLIATH REULR with and without treatment of 3C Protease. FIG. 6A provides a schematic model of a Receptor PD1-GOLIATH REULR concept. The REULR molecules were engineered with a 3C enzyme cleavage site that functions as the linker between the two binding modules between substrate and E3 Ligase. The inclusion of 3C protease during the assay would physically separate the link between the bispecific REULR molecule and neutralize the REULR activity. FIG. 6B provides enforced recruitment of GOLIATH to PD-1 using different version of PD-1-GOLIATH REULR molecules reduces PD-1 cell surface levels when treated with intact REULR. In contrast, PD-1-GOLIATH REULR molecules that were cleaved with 3C protease remain inactive due to loss of the bispecific nature of the REULR activity and show no effect on PD-1 surface levels.

### Example 4

HEK293F cells were transiently transfected with myc-tagged full length GOLIATH cDNA (hRNF130; human) and FLAG-tagged full length PD-1 cDNA (human). 48h post transfection, transfected cells were either incubated with PD1-GOLIATH Nanobody versions alone (AI-PD1, D1-PD1, PD1-D1; 0.25 μM) or preincubated with monomeric GOLIATH Nanobody (Ai, D1; 40× excess) for 0.5h as indicated. Cells were incubated for 6h (37° C.; 5% CO2) in the presence of Cycloheximide and subsequently washed (3×) before FACS analysis using FLAG-Tag (D6W5B) Rabbit mAb (Alexa Fluor 647 Conjugate). Data are mean+s.d. from n=3 replicates.

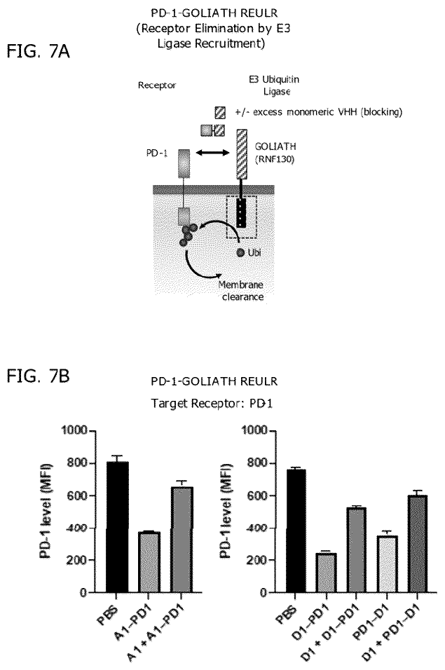

FIG. 7 depicts PD-1-GOLIATH REULR with and without blocking of the E3 Ligase using excess of monomeric GOLIATH VHH. FIG. 7A provides a schematic model of a Receptor PD1-GOLIATH REULR concept with and without pre-incubation of excess (40×) monomeric GOLIATH VHH. Enforced GOLIATH E3 ubiquitin Ligase recruitment to PD-1 reduces PD-1 cell surface levels by ubiquitination and subsequent membrane clearance by using intact REULR in comparison to GOLIATH neutralized with excess monomeric GOLIATH VHH. FIG. 7B provides results of enforced recruitment of GOLIATH to PD-1 using different version of PD-1-GOLIATH REULR molecules reduces PD-1 cell surface levels when treated with intact PD-1-GOLIATH REULR. To validate the specificity of PD-1 surface loss suing the PD-1-GOLIATH REULR, the GOLIATH receptor was blocked with excess of monomeric GOLIATH VHH before adding the intact PD-1-GOLIATH REULR and as a consequence rendered the addition of PD-1-GOLIATH REULR molecule inert, neutralizing the PD-1-GOLIATH REULR activity.

### Example 5

HEK293F-PD1 (Flag-PD-1) cells were transiently transfected with myc-tagged full length GOLIATH cDNA (hRNF130; human). 48h post transfection, transfected cells were either incubated with PD1-GOLIATH Nanobody versions as indicated (D1-PD1; 0.25 μM) or with 3C protease cleaved PD1-GOLIATH (D1-PD1) and treated with proteasomal inhibitor (MG132; 10 μM), Lysosomal inhibitor (Bafilomycin; 100 nM) or DMSO for 6h (37° C.; 5% CO2) in the presence of Cycloheximide. Cells were subsequently washed (ice cold PBS; 2×) before cell lysis and analyzed by western blot using the indicated primary antibodies.

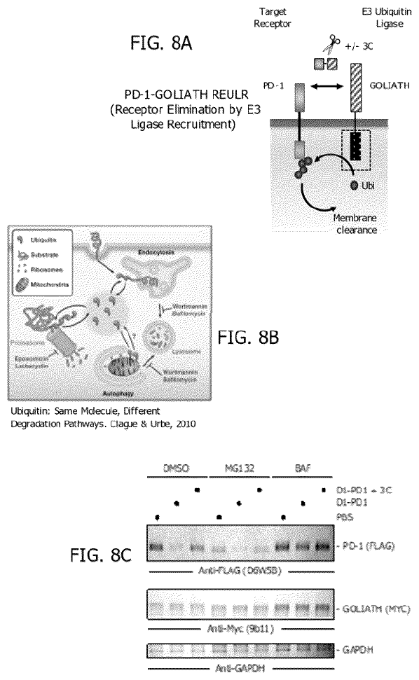

FIG. 8 depicts aspects of a PD1-GOLIATH REULR degradation pathway, specifically PD-1 degradation after treatment with PD-1-GOLIATH REULR in the presence or absence of proteasomal (MG132)l or lysosomal (Bafilomycin) degradation pathway inhibitors shows that PD-1 is degraded by the ubiquitin dependent lysosomal degradation pathway. FIG. 8A provides a schematic model of a Receptor PD1-GOLIATH REULR concept with and without pre-incubation of excess (40×) monomeric GOLIATH VHH. FIG. 8C depicts results of an experiment to evaluate the PD-1 degradation pathway after treatment with PD1-GOLIATH REULR, including pretreatment with either bafilomycin (i.e., a lysosome acidification inhibitor) or MG132 (i.e., a proteasome inhibitor). While pre-treatment with bafilomycin mitigated the degradation of PD-1, MG132 still resulted in PD-1 degradation after PD-1-GOLIATH REULR treatment, suggesting that the PD-1-GOLIATH REULR leads to PD-1 degradation by the ubiqitin dependent lysosomal pathway.

### Example 6

HEK293F cells were transiently transfected with myc-tagged full length GRAIL cDNA (hRNF128; human) and FLAG-tagged full length EGFR cDNA (human). 48h post transfection, transfected cells were incubated with different versions of EGFR (7D12) REULR molecules (7D12-E1, 7D12-E2, E1-7D12, E2-7D12; 0.5 μM) or EGFR (9g8) REULR versions (9g8-E1, 9g8-E2, E1-9g8, E2-9g8; 0.5 μM) as indicated. Cells were incubated for 6h (37° C.; 5% CO2) in the presence of Cycloheximide and subsequently washed (3×) before FACS analysis using FLAG-Tag (D6W5B) Rabbit mAb (Alexa Fluor 647 Conjugate). Data are mean+s.d. from n=3 replicates.

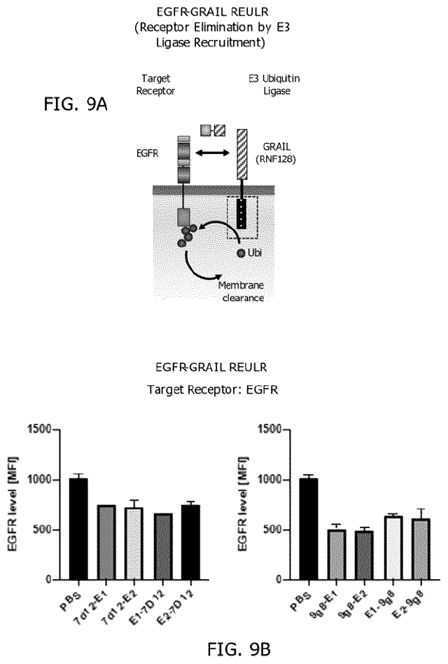

FIG. 9 depicts EGFR receptor elimination by EGFR-GRAIL REULR. FIG. 9A provides a schematic model of a Receptor EGFR-GRAIL REULR concept. FIG. 9B provides results of enforced recruitment of GRAIL to EGFR using different version of EGFR-GRAIL REULR molecules reduces EGFR cell surface levels when treated with intact EGFR-GRAIL REULR.

### Example 7

HEK293F cells were transiently transfected with myc-tagged full length GRAIL cDNA (hRNF128; human) and FLAG-tagged full length EGFR cDNA (human). 48h post transfection, transfected cells were incubated with different versions of EGFR—GRAIL REULR molecules (7D12-E2, 9g8-E2; 0.5 μM) or a PD1-GRAIL REULR (PD1-E2) that served as a negative control, as indicated. Cells were incubated for 6h (37° C.; 5% CO2) in the presence of Cycloheximide and subsequently washed (3×) before FACS analysis using FLAG-Tag (D6W5B) Rabbit mAb (Alexa Fluor 647 Conjugate). Data are mean+s.d. from n=3 replicates.

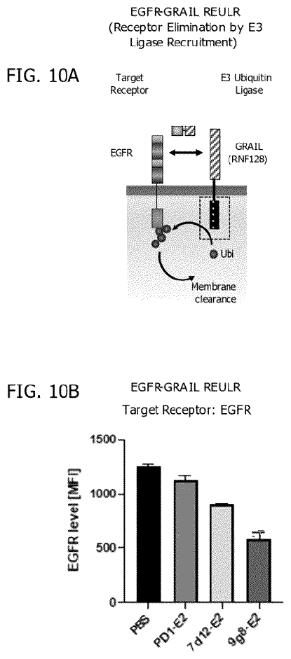

FIG. 10 depicts EGFR receptor elimination by EGFR-GRAIL REULR. FIG. 10A provides a schematic model of a Receptor EGFR-GRAIL REULR concept. FIG. 10B provides results of enforced recruitment of GRAIL to EGFR using different version of EGFR-GRAIL REULR molecules reduces EGFR cell surface levels when treated with intact EGFR-GRAIL REULR. By contrast, using a PD-1-GRAIL REULR shows no effect on EGFR receptor.

### Example 8

HEK293F cells were transiently transfected with myc-tagged full length GOLIATH cDNA (hRNF130; human) and FLAG-tagged full length EGFR cDNA (human). 48h post transfection, transfected cells were either incubated with EGFR-GOLIATH Nanobody versions as indicated (A1-7D12, D1-7D12, 7D12-D1; 0.5 μM) or with 3C protease cleaved EGFR-OLIATH versions for 6h (37° C.; 5% CO2) in the presence of Cycloheximide. Cells were subsequently washed (3×) before FACS analysis using FLAG-Tag (D6W5B) Rabbit mAb (Alexa Fluor 647 Conjugate). Data are mean+s.d. from n=3 replicates.

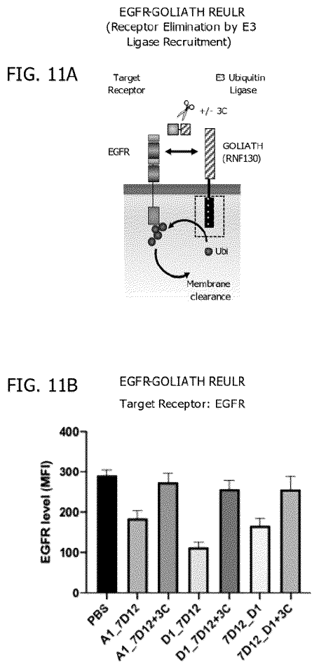

FIG. 11 depicts EGFR receptor elimination by EGFR-GOLIATH REULR with and without treatment of 3C Protease. FIG. 11A provides a schematic model of a Receptor EGFR-GOLIATH REULR concept. The REULR molecules were engineered with a 3C enzyme cleavage site that functions as the linker between the two binding modules between substrate and E3 Ligase. The inclusion of 3C protease during the assay would physically separate the link between the bispecific REULR molecule and neutralize the REULR activity. FIG. 11B depicts results of enforced recruitment of GOLIATH to EGFR using different version of EGFR-GOLIATH REULR molecules. EGFR receptor cell surface levels are reduced when treated with intact REULR. By contrast, EGFR-GOLIATH REULR molecules that were cleaved with 3C protease remain inactive due to loss of the bispecific nature of the REULR activity and show no effect on EGFR surface levels.

### Example 9

HEK293F cells were transiently transfected with myc-tagged full length GRAIL cDNA (hRNF128; human) and FLAG-tagged full length EpoR cDNA (human). 48h post transfection, transfected cells were either incubated with a EpoR-GRAIL REULR molecule or with 3C protease cleaved EpoR-GRAIL for 6h (37° C.; 5% CO2) in the presence of Cycloheximide. Cells were subsequently washed (3×) before FACS analysis using FLAG-Tag (D6W5B) Rabbit mAb (Alexa Fluor 647 Conjugate). Data are mean+s.d. from n=3 replicates.

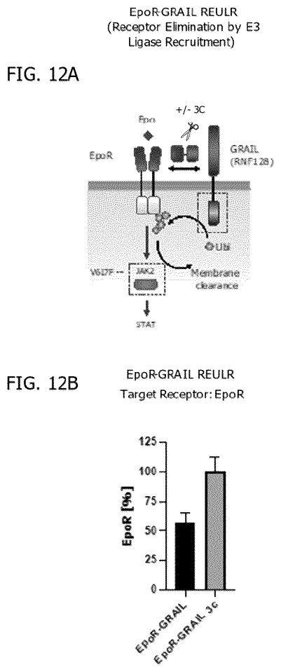

FIG. 12 depicts EpoR receptor elimination by a EpoR-GRAIL REULR with and without treatment of 3C Protease. FIG. 12A provides a schematic model of the Receptor EpoR-GRAIL REULR concept. The REULR molecules were engineered with a 3C enzyme cleavage site that functions as the linker between the two binding modules between substrate and E3 Ligase. The inclusion of 3C protease during the assay would physically separate the link between the bispecific REULR molecule and neutralize the REULR activity. FIG. 12B depicts results of enforced recruitment of GRAIL to EpoR using a EpoR-GRAIL REULR molecule. Treatment with intact EpoR-GRAIL REULR molecule reduces EpoR receptor cell surface levels. By contrast, EpoR-GRAIL REULR molecules that were cleaved with 3C protease remain inactive due to loss of the bispecific nature of the REULR activity and show no effect on EpoR surface levels.

### Example 10

HEK293F cells were transiently transfected with myc-tagged full length RNF43 cDNA (hRNF43; human) and FLAG-tagged full length EpoR cDNA (human). 48h post transfection, transfected cells were either incubated with a EpoR-RNF43 REULR molecule or with 3C protease cleaved EpoR-GRAIL for 6h (37° C.; 5% CO2) in the presence of Cycloheximide. Cells were subsequently washed (3×) before FACS analysis using FLAG-Tag (D6W5B) Rabbit mAb (Alexa Fluor 647 Conjugate). Data are mean+s.d. from n=3 replicates.

FIG. 13 depicts EpoR receptor elimination by a EpoR-RNF43 REULR with and without treatment of 3C Protease. FIG. 13A provides a schematic model of the Receptor EpoR-RNF43 REULR concept. The REULR molecules were engineered with a 3C enzyme cleavage site that functions as the linker between the two binding modules between substrate and E3 Ligase. The inclusion of 3C protease during the assay would physically separate the link between the bispecific REULR molecule and neutralize the REULR activity. FIG. 13B depicts results of enforced recruitment of RNF43 to EpoR using a EpoR-RNF43 REULR molecule. Treatment with intact EpoR-RNF43 REULR molecule reduces EpoR receptor cell surface levels. By contrast, EpoR-RNF43 REULR molecules that were cleaved with 3C protease remain inactive due to loss of the bispecific nature of the REULR activity and show no effect on EpoR surface levels.

### Example 11

HEK293F cells were transiently transfected with myc-tagged full length ZNRF3 cDNA (hZNRF3; human) and FLAG-tagged full length EpoR cDNA (human). 48h post transfection, transfected cells were either incubated with a EpoR-ZNRF3 REULR molecule or with 3C protease cleaved EpoR-ZNRF3 for 6h (37° C.; 5% CO2) in the presence of Cycloheximide. Cells were subsequently washed (3×) before FACS analysis using FLAG-Tag (D6W5B) Rabbit mAb (Alexa Fluor 647 Conjugate). Data are mean+s.d. from n=3 replicates.

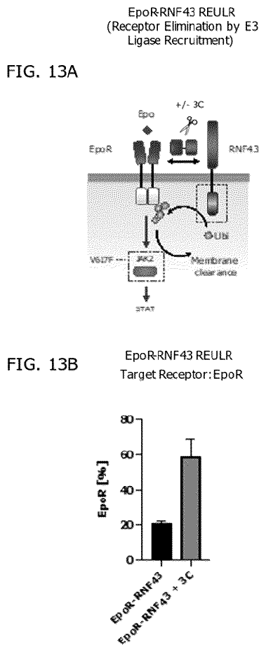

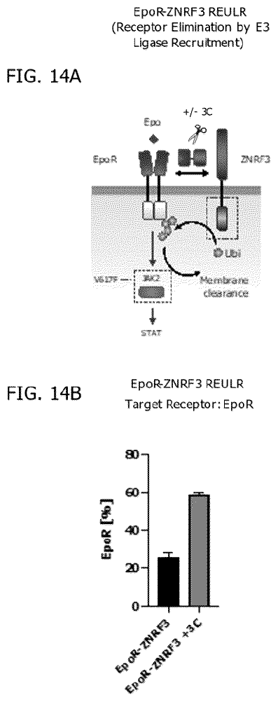

FIG. 14 depicts EpoR receptor elimination by a EpoR-ZNRF3 REULR with and without treatment of 3C Protease. FIG. 14A provides a schematic model of the Receptor EpoR-ZNRF3 REULR concept. The REULR molecules were engineered with a 3C enzyme cleavage site that functions as the linker between the two binding modules between substrate and E3 Ligase. The inclusion of 3C protease during the assay would physically separate the link between the bispecific REULR molecule and neutralize the REULR activity. FIG. 14B depicts results of enforced recruitment of ZNRF3 to EpoR using a EpoR-ZNRF3 REULR molecule. Treatment with intact EpoR-ZNRF3 REULR molecule reduces EpoR receptor cell surface levels. By contrast, EpoR-ZNRF3 REULR molecules that were cleaved with 3C protease remain inactive due to loss of the bispecific nature of the REULR activity and show no effect on EpoR surface levels.

### Example 12

HEK293F cells were transiently transfected with myc-tagged full length GRAIL cDNA (hRNF128; human). 48h post transfection, transfected cells were either incubated with GRAIL-GRAIL versions as indicated (E1-E1, E2-E2; 0.5 μM) or with 3C protease cleaved GRAIL-GRAIL versions for 6h (37° C.; 5% CO2). Cells were subsequently washed (3×) before FACS analysis using Myc-Tag (9B11) Mouse mAb (Alexa Fluor 647 Conjugate). Data are mean+s.d. from n=3 replicates.

FIG. 15 depicts GRAIL receptor elimination by GRAIL-GRAIL Fratricide REULR. FIG. 15A depicts a schematic model of a Ligase GRAIL-GRAIL Fratricide REULR concept. The REULR molecules were engineered with a 3C enzyme cleavage site that functions as the linker between the two binding modules between the two E3 Ligase. The inclusion of 3C protease during the assay would physically separate the link between the bispecific REULR molecule and neutralize the REULR activity. FIG. 15B depicts results of enforced recruitment of GRAIL to GRAIL by homodimerization and self-elimination using different version of GRAIL-GRAIL Fratricide REULR molecules. GRAIL cell surface levels are reduced when treated with intact GRAIL-GRAIL Fratricide REULR. In contrast, GRAIL-GRAIL Fratricide REULR molecules that were cleaved with 3C protease remain inactive due to loss of the bispecific nature of the REULR activity and show no effect on GRAIL receptor surface levels.

### Example 13

HEK293F cells were transiently transfected with myc-tagged full length GRAIL cDNA (hRNF128; human). 48h post transfection, transfected cells were either incubated with GRAIL-GRAIL Nanobody versions alone (E1-E1, E2-E2; 0.25 μM) or precincubated with monomeric GRAIL Nanobody (E1, E2; 40× excess) for 0.5h as indicated. Cells were incubated for 6h (37° C.; 5% CO2) in the presence of Cycloheximide and subsequently washed (3×) before FACS analysis using Myc-Tag (9B11) Mouse mAb (Alexa Fluor 647 Conjugate). Data are mean+s.d. from n=3 replicates.

FIG. 15C depicts results of enforced recruitment of GRAIL to itself using different version of GRAIL-GRAIL Fratricide REULR with and without preincubation with excess monomeric VHH. Intact, non blocked GRAIL-GRAIL Fratricide REULR molecules reduces GRAIL receptor cell surface levels. To validate the specificity of GRAIL receptor surface loss using the GRAIL-GRAIL Fratricide REULR, the GRAIL receptor was blocked with excess of monomeric GRAIL VHH before adding the intact GRAIL-GRAIL Fratricide REULR and as a consequence rendered the addition of GRAIL-GRAIL Fratricide REULR molecule inert, neutralizing the GRAIL-GRAIL Fratricide REULR activity.

### Example 14

HEK293F cells were transiently transfected with myc-tagged full length RNF43 cDNA (hRNF43; human). 48h post transfection, transfected cells were either incubated with intact RNF43-RNF43 Fratricide REULR molecule as indicated (0.5 μM) or with monomeric RNF43 VHH for 6h (37° C.; 5% CO2). Cells were subsequently washed (3×) before FACS analysis using Myc-Tag (9B111) Mouse mAb (Alexa Fluor 647 Conjugate). Data are mean+s.d. from n=3 replicates.

FIG. 16 depicts RNF43 receptor elimination by RNF43-RNF43 Fratricide REULR. FIG. 16A depicts a schematic model of a Ligase RNF43-RNF43 Fratricide REULR concept. FIG. 16B depicts results of enforced recruitment of RNF43 to itself using an intact RNF43-RNF43 Fratricide REULR molecule or monomeric RNF43 VHH. Treatment with intact RNF43-RNF43 Fratricide REULR molecules reduces RNF43 receptor cell surface levels.

### Example 15

HEK293F cells were transiently transfected with myc-tagged full length ZNRF3 cDNA (hZNRF3; human). 48h post transfection, transfected cells were either incubated with intact ZNRF3-ZNRF3 Fratricide REULR molecule as indicated (0.5 μM) or with monomeric ZNRF3 VHH for 6h (37° C.; 5% CO2). Cells were subsequently washed (3×) before FACS analysis using Myc-Tag (9B111) Mouse mAb (Alexa Fluor 647 Conjugate). Data are mean+s.d. from n=3 replicates.

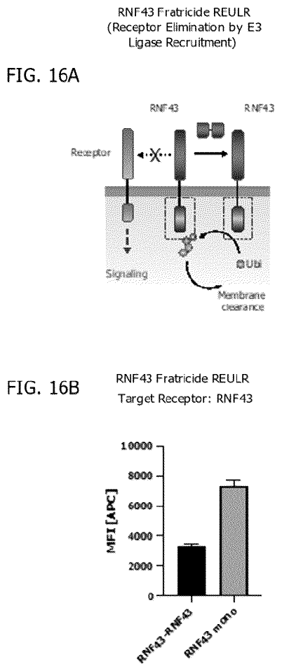

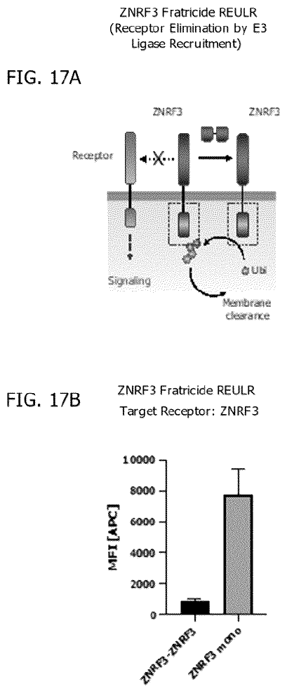

FIG. 17 depicts ZNRF3 receptor elimination by ZNRF3-ZNRF3 Fratricide REULR. FIG. 17A depicts a schematic model of a ZNRF3-ZNRF3 Fratricide REULR concept. FIG. 17B depicts results of enforced recruitment of ZNRF3 to itself using an intact ZNRF3-ZNRF3 Fratricide REULR molecule or monomeric ZNRF3 VHH. Treatment with intact ZNRF3-ZNRF3 Fratricide REULR molecules reduces ZNRF3 receptor cell surface levels.

### Example 16

HEK293F cells were transiently transfected with myc-tagged full length RNF43 cDNA (hRNF43; human). 48h post transfection, transfected cells were either incubated with intact RNF43-ZNRF3 Fratricide REULR molecule as indicated (0.5 μM) or with monomeric ZNRF3 and RNF43 VHH for 6h (37° C.; 5% CO2). Cells were subsequently washed (3×) before FACS analysis using Myc-Tag (9B11) Mouse mAb (Alexa Fluor 647 Conjugate). Data are mean+s.d. from n=3 replicates.

FIG. 18 depicts RNF43 receptor elimination by RNF43-ZNRF3 Fratricide REULR. FIG. 18A depicts a schematic model of a RNF43-ZNRF3 Fratricide REULR concept. FIG. 18B depicts results of enforced recruitment of ZNRF3 to RNF43 using an intact RNF43-ZNRF3 Fratricide REULR molecule or monomeric RNF43 and ZNRF3 VHH. Treatment with intact RNF43-ZNRF3 Fratricide REULR molecules reduces RNF43 receptor cell surface levels.

### Example 17

HEK293T STF cells were seeded at a density of 7.5 k cell/well. After 24h, cells were treated with PBS, WNT3aCM (WNT3a conditioned media; 10%), WNT3aCM (10%) plus various intact WNT-REULR Fratricide molecules targeting RNF43, ZNRF3 or RNF43 and ZNRF3 or monomeric RNF43 or ZNRF3 VHH. Cell were then incubated for 48h, washed and subjected to Luciferase reporter assay to measure WNT signaling activity normalized to PBS, dotted line represents the mean luciferase activity of Wnt3aCM. Data are mean+s.d. from n=3 replicates. WNT3aCM (WNT3a conditioned media) was prepared as previously described (Willert K, et al, Nature 423: 448-452, 2003. PubMed: 12717451).

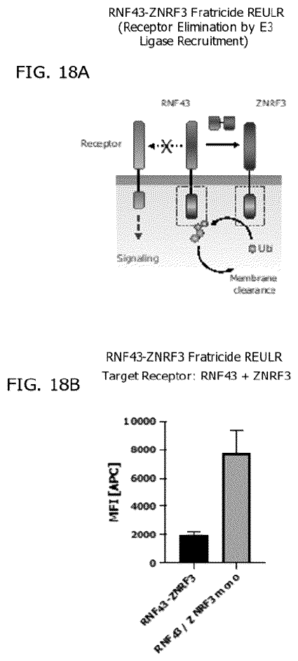

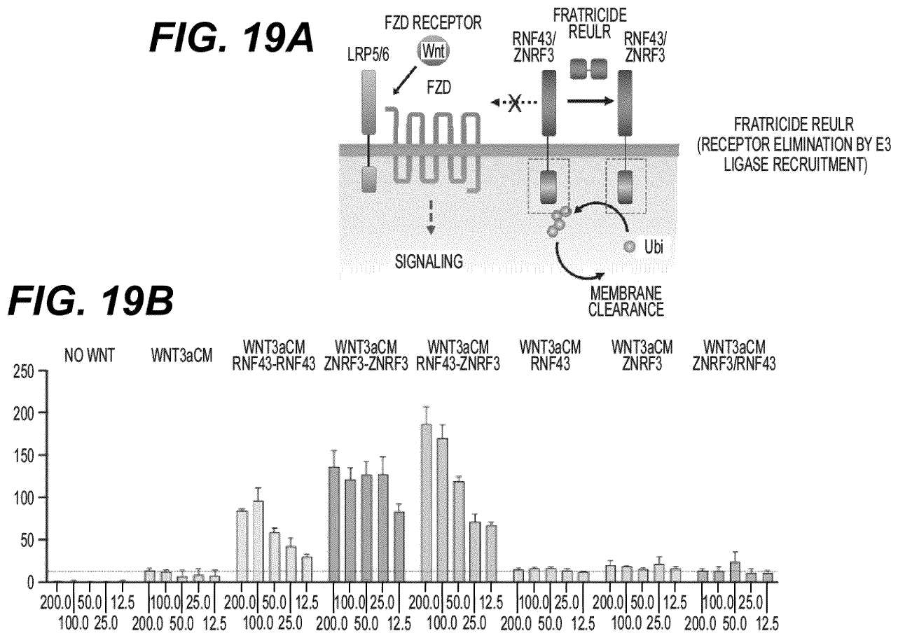

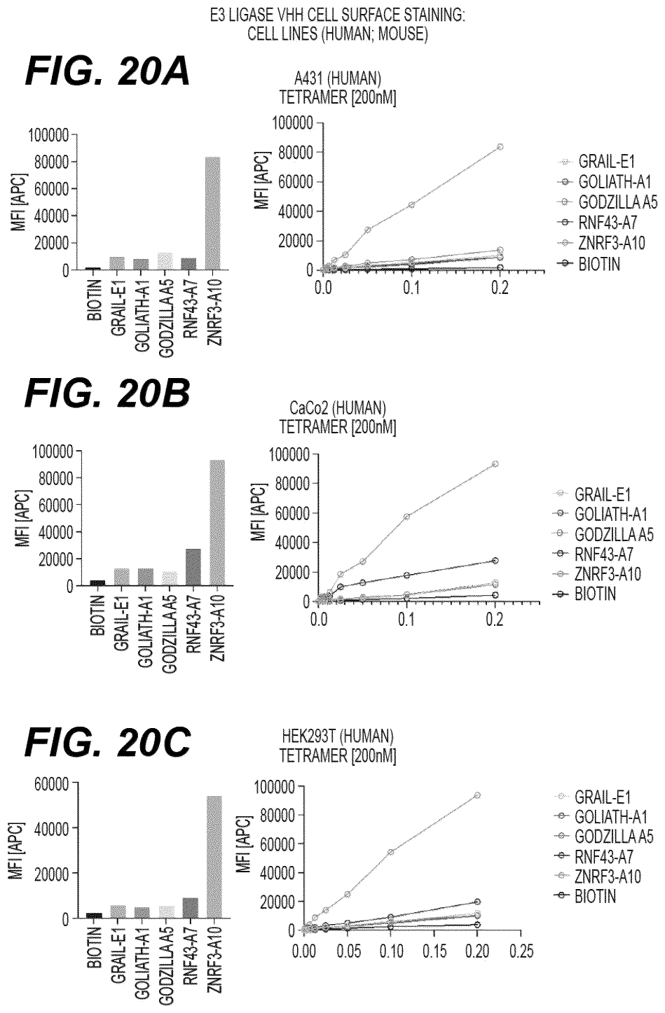

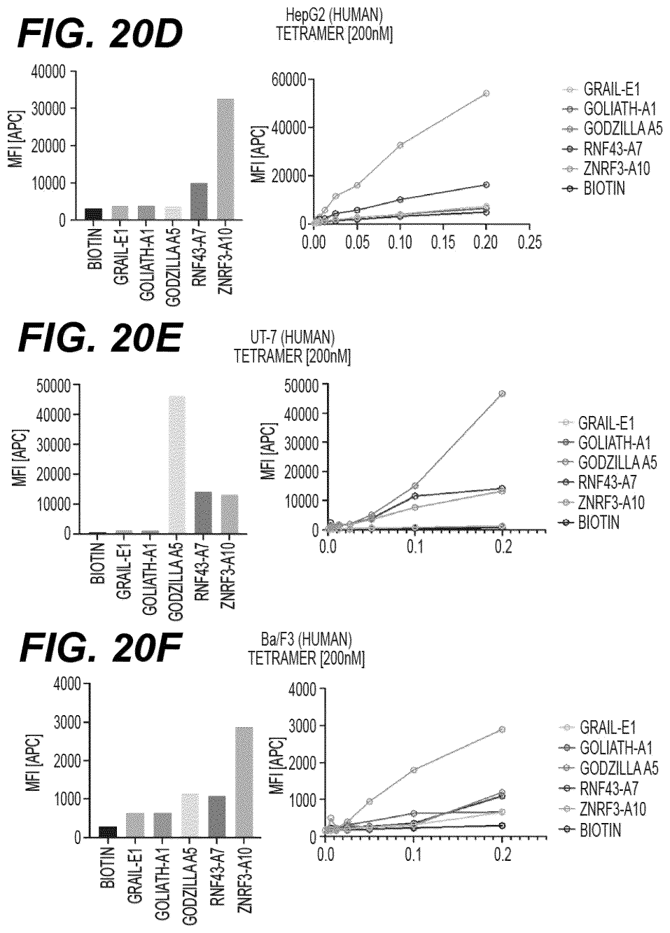

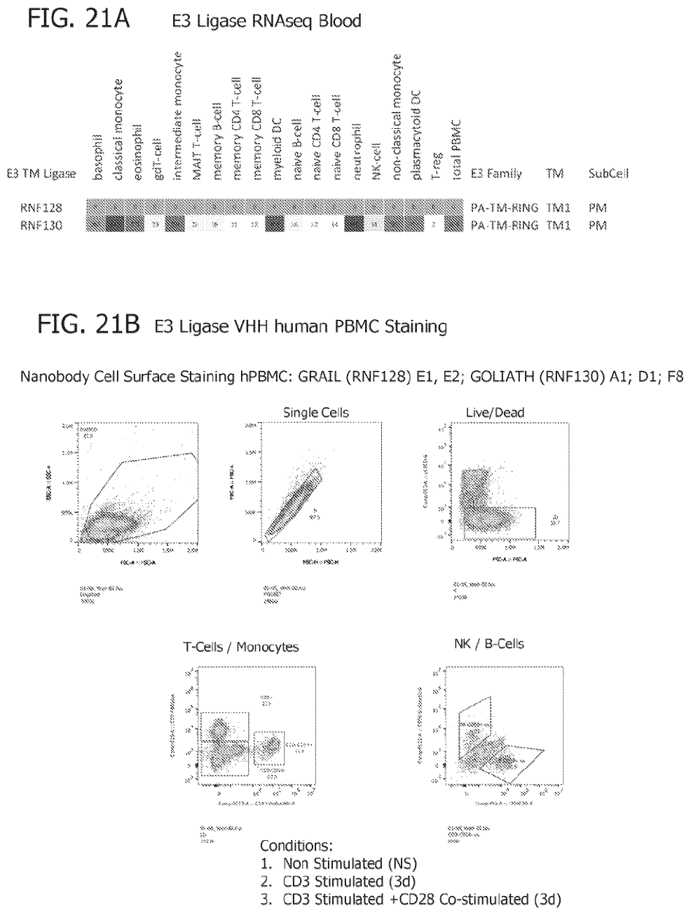

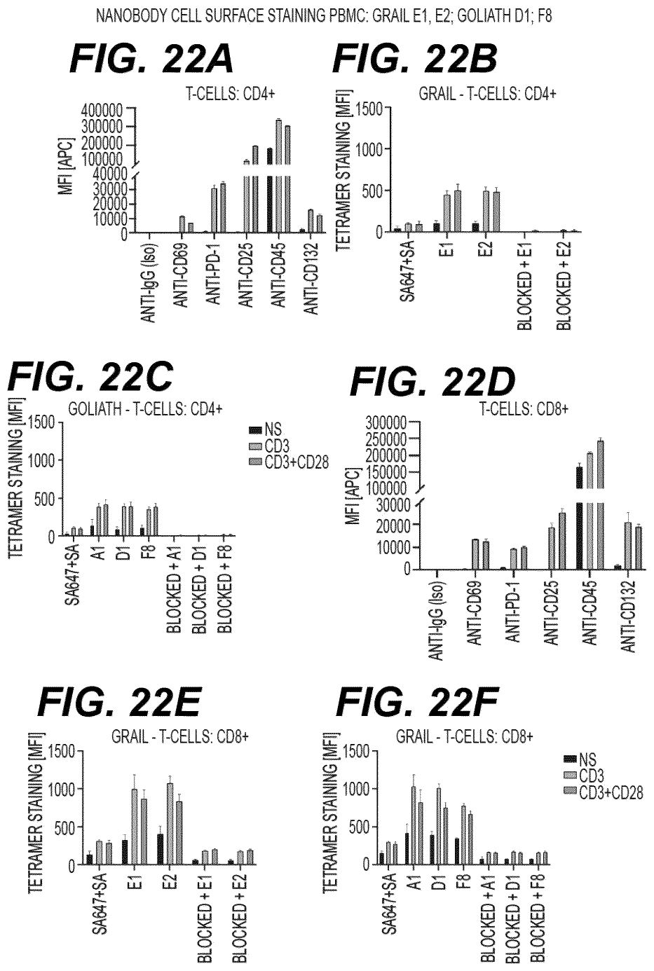

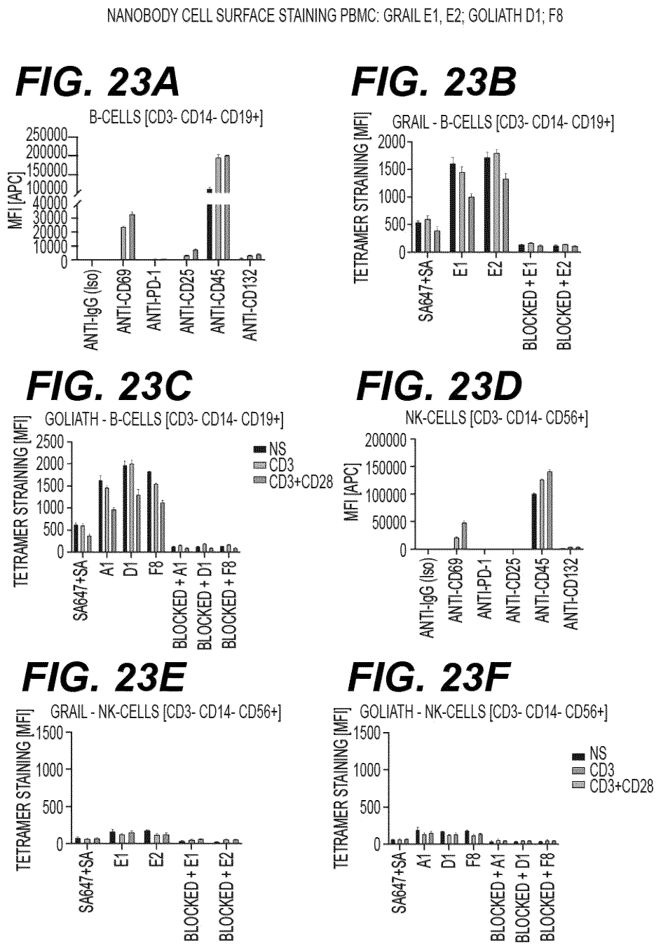

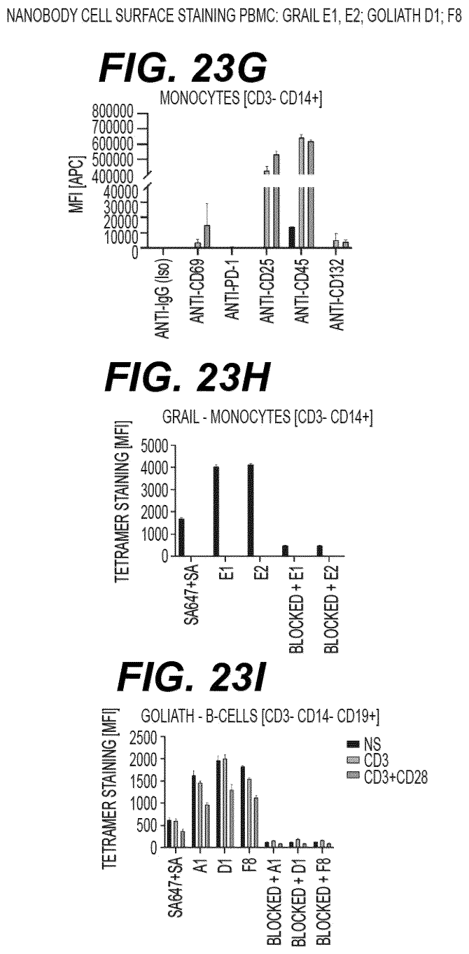

FIG. 19 depicts a WNT Signaling potentiation using various Fratricide REULR molecules targeting either RNF43, ZNRF3 or RNF43 and ZNRF3. FIG. 19A depicts a schematic model of a Fratricide REULR concept modulating the WNT Signaling pathway. FIG. B depicts results of enforced recruitment of RNF43 to RNF43, ZNRF3 to ZNRF3 or RNF43 to ZNRF3 using various intact RNF43 and ZNRF3 Fratricide REULR molecule or monomeric RNF43 and ZNRF3 VHH. Treatment with intact RNF43-RNF43, ZNRF3-ZNRF3 or RNF43-ZNRF3 Fratricide REULR molecules potentiates WNT Signaling in a Luciferase (STF) reporter assay measuring WNT signaling activity.

The above examples and embodiments are included for illustrative purposes only and are not intended to limit the scope of the disclosure. Many variations to those methods, systems, kits, and devices described above are possible. Since modifications and variations to the examples described above will be apparent to those of skill in this art, it is intended that this invention be limited only by the scope of the appended claims. All publications and references cited herein are expressly incorporated herein by reference in their entirety and/or for the specific reason for which they are cited herein.

1. A Receptor Elimination by E3 Ubiquitin Ligase Recruitment (REULR) construct compound, comprising a variable domain heavy chain antibody (VHH) capable of specially binding for a E3 Ubiquitin Ligase, and a VHH capable of specially binding a target protein comprising a cell surface protein, wherein the E3 Ubiquitin Ligase and the target protein are different proteins.

2. The REULR construct compound of claim 1, further comprising a linker molecule adapted to ligate the VHH specific for the E3 Ubiquitin Ligase to the VHH capable of specially binding a target protein in a manner such that the VHH capable of specially binding the E3 Ubiquitin Ligase and the VHH capable of specially binding a target protein are each independently available and able, respectively, to bind with the E3 Ubiquitin Ligase and the target protein.

3. The REULR construct compound of claim 1, wherein the E3 Ubiquitin Ligase is
(a) a RING type E3 Ubiquitin Ligase,
(b) GRAIL, GODZILLA, GOLIATH, RNF43 and/or ZNRF3, or
(c), selected from one or more RNF133, RNF148, RNF128, RNF149, RNF130, RNF150, RNF122, RNF43, ZNRF4, RNF13, RNF167, AMFR, STVN1, RNF170, RNF121, RNF175, RNF139, RNF145 MARCHF5, VFPL1, RNFT1, RNF180, RNF103, RNF5, RNF185, RNF19A, RNF19B, RNF144A, RNF144B, RNF217, MARCHF1, MARCHF8, MARCHF2, MARCHF3, MARCHF11, MARCHF4, MARCHF9 MARCHF6, NR1H4, RNF126, DCST1, RNF152, RNF186, CGRRF1, MUL1, TRIM13, TRIM59, RNF112.

4. The REULR construct compound of claim 1, further comprising one or more additional VHH capable of specially binding the E3 Ubiquitin Ligase, and/or one or more additional VHH capable of specially binding the target protein.

5. REULR construct compound of claim 1, wherein the REULR construct is defined by the following formula: 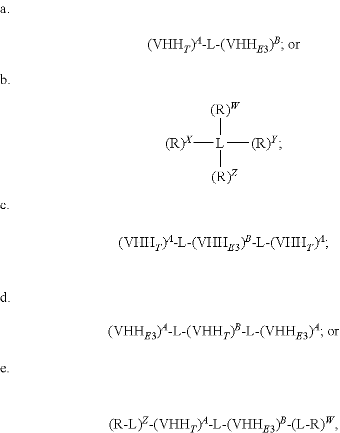
wherein VHH is a variable domain heavy chain antibody that specifically binds a target protein or an E3 Ubiquitin Ligase;
wherein R is VHH_{T} or VHH_{E3};
wherein L is a linker molecule;
wherein _{T} is a target protein such that the VHH is capable of specially binding the target protein;
wherein _{E3} is an E3 Ubiquitin Ligase such that the VHH is capable of specially binding the E3 Ubiquitin Ligase;
wherein ^{A} is 1 or more;
wherein ^{B} is 1 or more;
wherein ^{w} is 0, or 1 or more;
wherein ^{X} is 0, or 1 or more;
wherein ^{y} is 0, or 1 or more; and
wherein ^{z} is 0, or 1 or more.

6. (canceled)

7. The REULR construct compound of claim 5, wherein
(a) ^{A} is 2 and/or ^{B} is 2;
(b) ^{W} is 1, ^{y} is 1, ^{x} is 1, or ^{z} is 0;
(b) ^{W} is 1, ^{y} is 1, ^{x} is 1, or ^{z} is 1;
(c) ^{W} is 0, ^{y} is 2, ^{x} is 2, or ^{z} is 0;
(c) ^{W} is 0, ^{y} is 2, ^{x} is 2, or ^{z} is 1;
(e) ^{W} is 0, ^{y} is 1, ^{x} is 1, or ^{z} is 0;
(e) ^{W} is 0, ^{y} is 1, ^{x} is 1, or ^{z} is 1;
(i) ^{A} is 3 and/or ^{B} is 3.

8-14. (canceled)

15. The REULR construct composition of claim 5, wherein the linker molecule is adapted to ligate the VHH specific for the E3 Ubiquitin Ligase to the VHH capable of specially binding a target protein in a manner such that the VHH capable of specially binding the E3 Ubiquitin Ligase and the VHH capable of specially binding a target protein are independently available and able, respectively, to bind with the E3 Ubiquitin Ligase and the target protein.

16. (canceled)

17. The REULR construct of claim 1, wherein the cell surface protein is a receptor.

18. The REULR construct of claim 5, wherein the cell surface protein is associated with a cell proliferative disorder or disease.

19-21. (canceled)

22. An isolated nucleic acid encoding the REULR construct compound of claim 1.

23. A vector comprising the isolated nucleic acid of claim 22, operably linked with an expression control sequence.

24. A cell comprising the vector of claim 23.

25. A kit comprising the REULR construct compound of claim 1.

26. A method of modulating a target protein expressed on a cell, comprising contacting the cell expressing the cell surface protein with the REULR construct compound of claim 3, and permitting the REULR construct compound to interact with the cell surface protein and the E3 Ubiquitin Ligase, wherein binding of the REULR construct compound with the E3 Ubiquitin Ligase and the cell surface protein results in a modulation of the expression of the cell surface protein.

27. The method of claim 26, wherein the modulation comprises degrading expression of the cell surface protein or converting the cell surface protein to a Ubiquitin substrate recruitment domain.

28-45. (canceled)

46. A method of modulating a target protein expressed on a cell, comprising contacting the cell expressing the cell surface protein with the vector of claim 23, expressing the REULR construct compound, and permitting the REULR construct compound to interact with the cell surface protein and the E3 Ubiquitin Ligase, wherein binding of the REULR construct compound with the E3 Ubiquitin Ligase and the cell surface protein results in a modulation of the expression of the cell surface protein.

47. A pharmaceutical composition comprising the REULR compound of claim 1, formulated for use in treating a disease associated with the cell surface protein.

48. The pharmaceutical composition of claim 47, wherein
i. the E3 Ubiquitin Ligase is
(a) a RING type E3 Ubiquitin Ligase,
(b) GRAIL, GODZILLA, GOLIATH, RNF43 and/or ZNRF3, or
(c) selected from one or more of RNF133, RNF148, RNF128, RNF149, RNF130, RNF150, RNF122, RNF43, ZNRF3, ZNRF4, RNF13, RNF167, AMFR, STVN1, RNF170, RNF121, RNF175, RNF139, RNF145, MARCHF5, VFPL1, RNFT1, RNF180, RNF103, RNF182, RNF5, RNF185, RNF19A, RNF19B, RNF144A, RNF144B, RNF217, MARCHF1, MARCHF8, MARCHF2, MARCHF3, MARCHF11, MARCHF4, MARCHF9, MARCHF6, NR1H4, RNF126, DCST1, RNF152, RNF186, CGRRF1, MUL1, TRIM13, TRIM59, and RNF112; and
ii. the cell surface protein is
(a) associated with one or more of a cancer, a neurological disorder, a lymphoma, a leukemia, diabetes, an autoimmune disorder, a viral infection, a bacterial infection, or a parasitic infection, Alzheimer's disease, or heart disease,
(b) a receptor that is or is associated with a disease or disorder, oncogenic potential, an immune checkpoint, an innate/adaptive immunity, HIV, inflammation, autoimmunity, a tumor associated antigen, a tumor antigen, or
(c) a disease-associated cell surface protein that contains a ubiquitin acceptor site in an intracellular domain of the disease-associated protein.

49. A pharmaceutical composition comprising the isolated nucleic acid of claim 22, formulated for use in treating a disease associated with the cell surface protein, wherein
i. the E3 Ubiquitin Ligase is
(a) a RING type E3 Ubiquitin Ligase,
(b) GRAIL, GODZILLA, GOLIATH, RNF43 and/or ZNRF3, or
(c) selected from one or more of RNF133, RNF148, RNF128, RNF149, RNF130, RNF150, RNF122, RNF43, ZNRF3, ZNRF4, RNF13, RNF167, AMFR, STVN1, RNF170, RNF121, RNF175, RNF139, RNF145, MARCHF5, VFPL1, RNFT1, RNF180, RNF103, RNF182, RNF5, RNF185, RNF19A, RNF19B, RNF144A, RNF144B, RNF217, MARCHF1, MARCHF8, MARCHF2, MARCHF3, MARCHF11, MARCHF4, MARCHF9, MARCHF6, NR1H4, RNF126, DCST1, RNF152, RNF186, CGRRF1, MUL1, TRIM13, TRIM59, and RNF112; and
ii. the cell surface protein is
(a) associated with one or more of a cancer, a neurological disorder, a lymphoma, a leukemia, diabetes, an autoimmune disorder, a viral infection, a bacterial infection, or a parasitic infection, Alzheimer's disease, or heart disease,
(b) a receptor that is or is associated with a disease or disorder, oncogenic potential, an immune checkpoint, an innate/adaptive immunity, HIV, inflammation, autoimmunity, a tumor associated antigen, a tumor antigen, or
(c) a disease-associated cell surface protein that contains a ubiquitin acceptor site in an intracellular domain of the disease-associated protein.
